SEQUENCE LISTING (49 sequences)

SEQ ID NO: 1 - protein, 231 aa, synthetic construct
  REGION 1..231: note = Humanized anti-VEGF Fab heavy chain
  REGION 28..39: note = MISC_FEATURE - complementarity determining region
  REGION 54..83: note = MISC_FEATURE - complementarity determining region
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 2 - protein, 214 aa, synthetic construct
  REGION 1..214: note = Humanized anti-VEGF Fab
  REGION 26..37: note = MISC_FEATURE - copmlementarity determining region
  REGION 107..117: note = MISC_FEATURE - copmlementarity determining region
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 3957 bp, synthetic construct
  misc_feature 1..3957: note = 5'ITR.CB7.CI.aVEGFv2.rBG.3'ITR
  repeat_region 1..130: note = 5' ITR
  regulatory 198..579: note = promoter - CMV IE promoter; regulatory_class = promoter
  regulatory 279..538: note = enhancer - C4 enhancer with 2 mismatches; regulatory_class = enhancer
  misc_feature 279..538: note = C4 enhancer
  regulatory 582..862: note = promoter - CB promoter; regulatory_class = promoter
  regulatory 836..839: note = TATA box; regulatory_class = TATA_box
  misc_feature 955..1829: note = chicken beta-actin intron
  misc_feature 956..1928: note = Intron - chicken beta-actin intron
  misc_feature 956..1928: note = Intron - chicken beta-actin promoter
  5'UTR 1946..1993: note = c-myc 5' UTR
  misc_feature 1994..1999: note = kozak sequnce
  transit_peptide 1999..2058
  misc_feature 2752..2763: note = Furin cleavage site
  transit_peptide 2836..2895
  regulatory 3613..3739: note = polyA_signal - rabbit globin polyA; regulatory_class = polyA_signal_sequence
  repeat_region 3828..3957: note = 3' ITR
  source 1..3957: mol_type = other DNA
  CDS 2059..2427: note = aVEGFv2 VH
  CDS 2428..2748: note = CH1
  CDS 2764..2835: note = F2A linker
  CDS 2896..3216: note = aVEGFv2 VL
  CDS 3217..3537: note = CL
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagagctagcgggcactttgcactggaacttacaacacccgagcaaggacgcgactctccaccatgtatcggatgcagctgctgctgctgattgctctgagcctggctctggtgaccaacagcgaagtgcagctggtggaaagcggcggcggcctggtgcagcccggcggcagcctgcggctgagctgtgctgctagcggctatgactttacccattatggcatgaactgggtgcggcaggctcccggcaagggcctggaatgggtgggctggattaacacctataccggcgaacccacctatgctgctgactttaagcggcggtttacctttagcctggacaccagcaagagcaccgcttatctgcagatgaacagcctgcgggctgaagacaccgctgtgtattattgtgctaagtatccctattattatggcaccagccattggtattttgacgtgtggggccagggcaccctggtgaccgtgagcagcgctagcaccaagggccccagcgtgtttcccctggctcccagcagcaagagcaccagcggcggcaccgctgctctgggctgtctggtgaaggactattttcccgaacccgtgaccgtgagctggaacagcggcgctctgaccagcggcgtgcatacctttcccgctgtgctgcagagcagcggcctgtatagcctgagcagcgtggtgaccgtgcccagcagcagcctgggcacccagacctatatttgtaacgtgaaccataagcccagcaacaccaaggtggacaagaaggtggaacccaagagctgtgacaagacccatctgcggaagcggcgggctcccgtgaagcagaccctgaactttgacctgctgaagctggctggcgacgtggaaagcaaccccggccccatgtatcggatgcagctgctgctgctgattgctctgagcctggctctggtgaccaacagcgacattcagctgacccagagccccagcagcctgagcgctagcgtgggcgaccgggtgaccattacctgtagcgctagccaggacattagcaactatctgaactggtatcagcagaagcccggcaaggctcccaaggtgctgatttattttaccagcagcctgcatagcggcgtgcccagccggtttagcggcagcggcagcggcaccgactttaccctgaccattagcagcctgcagcccgaagactttgctacctattattgtcagcagtatagcaccgtgccctggacctttggccagggcaccaaggtggaaattaagcggaccgtggctgctcccagcgtgtttatttttccccccagcgacgaacagctgaagagcggcaccgctagcgtggtgtgtctgctgaacaacttttatccccgggaagctaaggtgcagtggaaggtggacaacgctctgcagagcggcaacagccaggaaagcgtgaccgaacaggacagcaaggacagcacctatagcctgagcagcaccctgaccctgagcaaggctgactatgaaaagcataaggtgtatgcttgtgaagtgacccatcagggcctgagcagccccgtgaccaagagctttaaccggggcgaatgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - protein, 123 aa, synthetic construct [sequence omitted]

SEQ ID NO: 5 - protein, 107 aa, synthetic construct
  REGION 1..107: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 6 - protein, 24 aa, synthetic construct
  REGION 1..24: note = Synthetic Construct
APVKQTLNFDLLKLAGDVESNPGP

SEQ ID NO: 7 - protein, 107 aa, synthetic construct [sequence omitted]

SEQ ID NO: 8 - protein, 107 aa, synthetic construct [sequence omitted]

SEQ ID NO: 9 - DNA, 3770 bp, synthetic construct
  misc_feature 1..3770: note = AAV25'ITR.UbC.Ci.aVEGFv2.rBG.AAV23'ITR
  repeat_region 17..146: note = 5' ITR
  regulatory 207..1435: note = promoter - UbC with C insertion at 289 and G insertion at 990; regulatory_class = promoter
  misc_feature 1529..1661: note = Intron - chimeric intron
  5'UTR 1736..1783: note = c-myc 5'UTR
  misc_feature 1784..1789: note = kozak
  misc_feature 1789..1848: note = kozak
  misc_feature 2542..2553: note = furin cleavage site
  misc_feature 2554..2625: note = F2a linker
  misc_feature 2626..2685: note = Leader
  regulatory 3345..3576: note = polyA_signal - SV40 late polyadenylation signal; regulatory_class = polyA_signal_sequence
  repeat_region 3641..3770: note = 3' ITR
  source 1..3770: mol_type = other DNA
  CDS 1849..2217: note = aVEGFv2 VH
  CDS 2218..2538: note = CH1
  CDS 2686..3006: note = aVEGFv2 VL
  CDS 3007..3327: note = CL
taaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagagctagcgggcactttgcactggaacttacaacacccgagcaaggacgcgactctccaccatgtatcggatgcagctgctgctgctgattgctctgagcctggctctggtgaccaacagcgaagtgcagctggtggaaagcggcggcggcctggtgcagcccggcggcagcctgcggctgagctgtgctgctagcggctatgactttacccattatggcatgaactgggtgcggcaggctcccggcaagggcctggaatgggtgggctggattaacacctataccggcgaacccacctatgctgctgactttaagcggcggtttacctttagcctggacaccagcaagagcaccgcttatctgcagatgaacagcctgcgggctgaagacaccgctgtgtattattgtgctaagtatccctattattatggcaccagccattggtattttgacgtgtggggccagggcaccctggtgaccgtgagcagcgctagcaccaagggccccagcgtgtttcccctggctcccagcagcaagagcaccagcggcggcaccgctgctctgggctgtctggtgaaggactattttcccgaacccgtgaccgtgagctggaacagcggcgctctgaccagcggcgtgcatacctttcccgctgtgctgcagagcagcggcctgtatagcctgagcagcgtggtgaccgtgcccagcagcagcctgggcacccagacctatatttgtaacgtgaaccataagcccagcaacaccaaggtggacaagaaggtggaacccaagagctgtgacaagacccatctgcggaagcggcgggctcccgtgaagcagaccctgaactttgacctgctgaagctggctggcgacgtggaaagcaaccccggccccatgtatcggatgcagctgctgctgctgattgctctgagcctggctctggtgaccaacagcgacattcagctgacccagagccccagcagcctgagcgctagcgtgggcgaccgggtgaccattacctgtagcgctagccaggacattagcaactatctgaactggtatcagcagaagcccggcaaggctcccaaggtgctgatttattttaccagcagcctgcatagcggcgtgcccagccggtttagcggcagcggcagcggcaccgactttaccctgaccattagcagcctgcagcccgaagactttgctacctattattgtcagcagtatagcaccgtgccctggacctttggccagggcaccaaggtggaaattaagcggaccgtggctgctcccagcgtgtttatttttccccccagcgacgaacagctgaagagcggcaccgctagcgtggtgtgtctgctgaacaacttttatccccgggaagctaaggtgcagtggaaggtggacaacgctctgcagagcggcaacagccaggaaagcgtgaccgaacaggacagcaaggacagcacctatagcctgagcagcaccctgaccctgagcaaggctgactatgaaaagcataaggtgtatgcttgtgaagtgacccatcagggcctgagcagccccgtgaccaagagctttaaccggggcgaatgttgataaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - protein, 123 aa, synthetic construct [sequence omitted]

SEQ ID NO: 11 - protein, 107 aa, synthetic construct; identical to SEQ ID NO: 5
  REGION 1..107: note = Synthetic Construct

SEQ ID NO: 12 - protein, 107 aa, synthetic construct [sequence omitted]

SEQ ID NO: 13 - protein, 107 aa, synthetic construct [sequence omitted]

SEQ ID NO: 14 - DNA, 3951 bp, synthetic construct
  misc_feature 1..3951: note = ITR.CB7.CI.aVEGRv3.rBG.ITR
  repeat_region 1..130: note = 5'ITR
  regulatory 198..579: note = promoter - CMV IE promoter; regulatory_class = promoter
  regulatory 279..538: note = enhancer - C4 enhancer with 2 mismatches; regulatory_class = enhancer
  regulatory 582..862: note = promoter - CB promoter; regulatory_class = promoter
  regulatory 836..839: note = TATA box; regulatory_class = TATA_box
  misc_feature 956..1928: note = Intron - chicken beta-actin intron
  5'UTR 1940..1987
  misc_feature 1988..1993: note = Kozak
  misc_feature 1993..2052: note = leader
  misc_feature 2746..2757: note = furin cleavage site
  misc_feature 2758..2829: note = F2a linker
  misc_feature 2830..2889: note = leader
  regulatory 3607..3733: note = polyA_signal - rabbit globin polyA; regulatory_class = polyA_signal_sequence
  repeat_region 3822..3951: note = 3' ITR
  source 1..3951: mol_type = other DNA
  CDS 2053..2421: note = aVEGFv3 VH
  CDS 2422..2742: note = CH1
  CDS 2890..3210: note = aVEGFv3 VL
  CDS 3211..3531: note = CL
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccgtatgcagctgctgctgctgatcgcactgtcactggcactggttaccaactcagaagttcagctggttgaatcaggcggcggcctggttcagcccggcggctcactgcgtctgtcatgtgcagcatcaggctacgatttcacccactacggcatgaactgggttcgtcaggcacccggcaaaggcctggaatgggttggctggatcaacacctacaccggcgaacccacctacgcagcagatttcaaacgtcgtttcaccttctcactggatacctcaaaatcaaccgcatacctgcagatgaactcactgcgtgcagaagataccgcagtttactactgtgcaaaatacccctactactacggcacctcacactggtacttcgatgtttggggccagggcaccctggttaccgtttcatcagcatcaaccaaaggcccctcagttttccccctggcaccctcatcaaaatcaacctcaggcggcaccgcagcactgggctgtctggttaaagattacttccccgaacccgttaccgtttcatggaactcaggcgcactgacctcaggcgttcacaccttccccgcagttctgcagtcatcaggcctgtactcactgtcatcagttgttaccgttccctcatcatcactgggcacccagacctacatctgtaacgttaaccacaaaccctcaaacaccaaagttgataaaaaagttgaacccaaatcatgtgataaaacccacctgcgtaaacgtcgtgcacccgttaaacagaccctgaacttcgatctgctgaaactggcaggcgatgttgaatcaaaccccggccccatgtaccgtatgcagctgctgctgctgatcgcactgtcactggcactggttaccaactcagatatccagctgacccagtcaccctcatcactgtcagcatcagttggcgatcgtgttaccatcacctgttcagcatcacaggatatctcaaactacctgaactggtaccagcagaaacccggcaaagcacccaaagttctgatctacttcacctcatcactgcactcaggcgttccctcacgtttctcaggctcaggctcaggcaccgatttcaccctgaccatctcatcactgcagcccgaagatttcgcaacctactactgtcagcagtactcaaccgttccctggaccttcggccagggcaccaaagttgaaatcaaacgtaccgttgcagcaccctcagttttcatcttccccccctcagatgaacagctgaaatcaggcaccgcatcagttgtttgtctgctgaacaacttctacccccgtgaagcaaaagttcagtggaaagttgataacgcactgcagtcaggcaactcacaggaatcagttaccgaacaggattcaaaagattcaacctactcactgtcatcaaccctgaccctgtcaaaagcagattacgaaaaacacaaagtttacgcatgtgaagttacccaccagggcctgtcatcacccgttaccaaatcattcaaccgtggcgaatgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - protein, 123 aa, synthetic construct [sequence omitted]

SEQ ID NO: 16 - protein, 107 aa, synthetic construct; identical to SEQ ID NO: 5
  REGION 1..107: note = Synthetic Construct

SEQ ID NO: 17 - protein, 107 aa, synthetic construct [sequence omitted]

SEQ ID NO: 18 - protein, 107 aa, synthetic construct [sequence omitted]

SEQ ID NO: 19 - DNA, 3763 bp, synthetic construct
  misc_feature 1..3763: note = ITR.UbC.PI.aVEGFv3.SV40.ITR
  repeat_region 17..146: note = AAV2 5' ITR
  regulatory 207..1434: note = promoter - UbC, with C insert at 289 and G insert at 990; regulatory_class = promoter
  misc_feature 1528..1660: note = Intron - chimeric intron
  5'UTR 1729..1776: note = c-myc 5'UTR
  misc_feature 1777..1782: note = kozak
  misc_feature 1782..1841: note = leader
  misc_feature 2535..2546: note = furin cleavage site
  misc_feature 2547..2618: note = F2a linker
  misc_feature 2619..2678: note = leader
  regulatory 3338..3569: note = polyA_signal - SV40 late polyA; regulatory_class = polyA_signal_sequence
  repeat_region 3634..3763: note = AAV2 3'ITR
  source 1..3763: mol_type = other DNA
  CDS 1842..2210: note = aVEGFv3 VH
  CDS 2211..2531: note = CH1
  CDS 2679..2999: note = aVEGFv3 VL
  CDS 3000..3320: note = CL
taaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgggcactttgcactggaacttacaacacccgagcaaggacgcgactctccaccatgtaccgtatgcagctgctgctgctgatcgcactgtcactggcactggttaccaactcagaagttcagctggttgaatcaggcggcggcctggttcagcccggcggctcactgcgtctgtcatgtgcagcatcaggctacgatttcacccactacggcatgaactgggttcgtcaggcacccggcaaaggcctggaatgggttggctggatcaacacctacaccggcgaacccacctacgcagcagatttcaaacgtcgtttcaccttctcactggatacctcaaaatcaaccgcatacctgcagatgaactcactgcgtgcagaagataccgcagtttactactgtgcaaaatacccctactactacggcacctcacactggtacttcgatgtttggggccagggcaccctggttaccgtttcatcagcatcaaccaaaggcccctcagttttccccctggcaccctcatcaaaatcaacctcaggcggcaccgcagcactgggctgtctggttaaagattacttccccgaacccgttaccgtttcatggaactcaggcgcactgacctcaggcgttcacaccttccccgcagttctgcagtcatcaggcctgtactcactgtcatcagttgttaccgttccctcatcatcactgggcacccagacctacatctgtaacgttaaccacaaaccctcaaacaccaaagttgataaaaaagttgaacccaaatcatgtgataaaacccacctgcgtaaacgtcgtgcacccgttaaacagaccctgaacttcgatctgctgaaactggcaggcgatgttgaatcaaaccccggccccatgtaccgtatgcagctgctgctgctgatcgcactgtcactggcactggttaccaactcagatatccagctgacccagtcaccctcatcactgtcagcatcagttggcgatcgtgttaccatcacctgttcagcatcacaggatatctcaaactacctgaactggtaccagcagaaacccggcaaagcacccaaagttctgatctacttcacctcatcactgcactcaggcgttccctcacgtttctcaggctcaggctcaggcaccgatttcaccctgaccatctcatcactgcagcccgaagatttcgcaacctactactgtcagcagtactcaaccgttccctggaccttcggccagggcaccaaagttgaaatcaaacgtaccgttgcagcaccctcagttttcatcttccccccctcagatgaacagctgaaatcaggcaccgcatcagttgtttgtctgctgaacaacttctacccccgtgaagcaaaagttcagtggaaagttgataacgcactgcagtcaggcaactcacaggaatcagttaccgaacaggattcaaaagattcaacctactcactgtcatcaaccctgaccctgtcaaaagcagattacgaaaaacacaaagtttacgcatgtgaagttacccaccagggcctgtcatcacccgttaccaaatcattcaaccgtggcgaatgttgataaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - protein, 123 aa, synthetic construct [sequence omitted]

SEQ ID NO: 21 - protein, 107 aa, synthetic construct; identical to SEQ ID NO: 5
  REGION 1..107: note = Synthetic Construct

SEQ ID NO: 22 - protein, 107 aa, synthetic construct [sequence omitted]

SEQ ID NO: 23 - protein, 107 aa, synthetic construct [sequence omitted]

SEQ ID NO: 24 - DNA, 3764 bp, synthetic construct
  misc_feature 1..3764: note = ITR.UbC.PI.aVEGFv1.SV40.ITR
  repeat_region 17..146: note = ITR
  regulatory 207..1435: note = promoter - UbC; regulatory_class = promoter
  misc_feature 1529..1661: note = Intron - Promoga chimeric intron
  5'UTR 1730..1777: note = c-myc 5'UTR
  misc_feature 1778..1783: note = kozak
  misc_feature 1783..1842: note = leader
  misc_feature 2536..2547: note = furin cleavage site
  misc_feature 2548..2619: note = F2A linker
  misc_feature 2620..2679: note = leader
  regulatory 3339..3570: note = polyA_signal - SV40 polyadenylation signal; regulatory_class = polyA_signal_sequence
  repeat_region 3635..3764: note = ITR
  source 1..3764: mol_type = other DNA
  CDS 1843..2211: note = aVEGFv1 VH
  CDS 2212..2532: note = CH1
  CDS 2680..3000: note = aVEGFv1 VL
  CDS 3001..3321: note = CL
taaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgggcactttgcactggaacttacaacacccgagcaaggacgcgactctccaccatgtaccgaatgcagctgctgctgctgatcgcactgagcctggcactggtgaccaacagcgaggtgcagctggtggagagcggaggaggactggtgcagccaggaggaagcctgcgactgagctgtgcagcaagcggatacgacttcacccattacggaatgaactgggtgcgacaggcaccaggaaagggactggagtgggtgggatggatcaacacctacaccggagagccaacctacgcagcagacttcaagcgacgattcaccttcagcctggacaccagcaagagcaccgcatacctgcagatgaacagcctgcgagcagaggacaccgcagtgtactactgtgcaaagtacccatactactacggaaccagccattggtacttcgacgtgtggggacagggaaccctggtgaccgtgagcagcgcaagcaccaagggaccaagcgtgttcccactggcaccaagcagcaagagcaccagcggaggaaccgcagcactgggatgtctggtgaaggactacttcccagagccagtgaccgtgagctggaacagcggagcactgaccagcggagtgcataccttcccagcagtgctgcagagcagcggactgtacagcctgagcagcgtggtgaccgtgccaagcagcagcctgggaacccagacctacatctgtaacgtgaaccataagccaagcaacaccaaggtggacaagaaggtggagccaaagagctgtgacaagacccatctgcgaaagcgacgagcaccagtgaagcagaccctgaacttcgacctgctgaagctggcaggagacgtggagagcaacccaggaccaatgtaccgaatgcagctgctgctgctgatcgcactgagcctggcactggtgaccaacagcgacatccagctgacccagagcccaagcagcctgagcgcaagcgtgggagaccgagtgaccatcacctgtagcgcaagccaggacatcagcaactacctgaactggtaccagcagaagccaggaaaggcaccaaaggtgctgatctacttcaccagcagcctgcatagcggagtgccaagccgattcagcggaagcggaagcggaaccgacttcaccctgaccatcagcagcctgcagccagaggacttcgcaacctactactgtcagcagtacagcaccgtgccatggaccttcggacagggaaccaaggtggagatcaagcgaaccgtggcagcaccaagcgtgttcatcttcccaccaagcgacgagcagctgaagagcggaaccgcaagcgtggtgtgtctgctgaacaacttctacccacgagaggcaaaggtgcagtggaaggtggacaacgcactgcagagcggaaacagccaggagagcgtgaccgagcaggacagcaaggacagcacctacagcctgagcagcaccctgaccctgagcaaggcagactacgagaagcataaggtgtacgcatgtgaggtgacccatcagggactgagcagcccagtgaccaagagcttcaaccgaggagagtgttgataaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - protein, 123 aa, synthetic construct [sequence omitted]

SEQ ID NO: 26 - protein, 107 aa, synthetic construct; identical to SEQ ID NO: 5
  REGION 1..107: note = Synthetic Construct

SEQ ID NO: 27 - protein, 107 aa, synthetic construct [sequence omitted]

SEQ ID NO: 28 - protein, 107 aa, synthetic construct [sequence omitted]

SEQ ID NO: 29 - protein, 20 aa, Homo sapiens
MYRMQLLSCIALSLALVTNS

SEQ ID NO: 30 - protein, 20 aa, synthetic construct
  REGION 1..20: note = synthetic leader
MYRMQLLLLIALSLALVTNS

SEQ ID NO: 31 - protein, 19 aa, synthetic construct
  REGION 1..19: note = synthetic leader 2
MRMQLLLLIALSLALVTNS

SEQ ID NO: 32 - DNA, 355 bp, synthetic construct
  misc_feature 1..355: note = derived from encephalomycarditts virus
  source 1..355: mol_type = other DNA
tatgctagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctctagtttcactttccctagtttcactttccctagtttcactttccctagtttcactttccctagtttcactttcccctcgaggatatcaagatctggcctcggcggccag

SEQ ID NO: 33 - protein, 513 aa, synthetic construct
  REGION 1..513: note = aVEGF
  REGION 1..20: note = MISC_FEATURE - leader
  REGION 21..252: note = MISC_FEATURE - aVEGF Heavy Chain
  REGION 280..299: note = MISC_FEATURE - leader
  REGION 300..513: note = MISC_FEATURE - aVEGF Light Chain
MYRMQLLLLIALSLALVTNSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLRKRRAPVKQTLNFDLLKLAGDVESNPGPMYRMQLLLLIALSLALVTNSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 3951 bp, synthetic construct
  misc_feature 1..3951: note = ITR.CB7.CI.aVEGFv1.rBG.ITR
  repeat_region 1..130: note = 5' ITR
  regulatory 204..584: note = promoter - CMV IE promoter; regulatory_class = promoter
  regulatory 585..862: note = promoter - CB promoter; regulatory_class = promoter
  regulatory 836..839: note = TATA box; regulatory_class = TATA_box
  misc_feature 956..1928: note = Intron - chicken beta-actin intron
  5'UTR 1940..1987: note = c-myc 5'UTR
  misc_feature 1988..1993: note = kozak
  misc_feature 1993..2052: note = leader
  misc_feature 2053..2421: note = aVEGFv1 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv1 VL
  misc_feature 3211..3531: note = CL
  misc_feature 3532..3537: note = stop cassette
  regulatory 3607..3733: note = polyA_signal - Rabbit globin poly A; regulatory_class = polyA_signal_sequence
  misc_feature 3785..3821: note = part of AAV
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccgaatgcagctgctgctgctgatcgcactgagcctggcactggtgaccaacagcgaggtgcagctggtggagagcggaggaggactggtgcagccaggaggaagcctgcgactgagctgtgcagcaagcggatacgacttcacccattacggaatgaactgggtgcgacaggcaccaggaaagggactggagtgggtgggatggatcaacacctacaccggagagccaacctacgcagcagacttcaagcgacgattcaccttcagcctggacaccagcaagagcaccgcatacctgcagatgaacagcctgcgagcagaggacaccgcagtgtactactgtgcaaagtacccatactactacggaaccagccattggtacttcgacgtgtggggacagggaaccctggtgaccgtgagcagcgcaagcaccaagggaccaagcgtgttcccactggcaccaagcagcaagagcaccagcggaggaaccgcagcactgggatgtctggtgaaggactacttcccagagccagtgaccgtgagctggaacagcggagcactgaccagcggagtgcataccttcccagcagtgctgcagagcagcggactgtacagcctgagcagcgtggtgaccgtgccaagcagcagcctgggaacccagacctacatctgtaacgtgaaccataagccaagcaacaccaaggtggacaagaaggtggagccaaagagctgtgacaagacccatctgcgaaagcgacgagcaccagtgaagcagaccctgaacttcgacctgctgaagctggcaggagacgtggagagcaacccaggaccaatgtaccgaatgcagctgctgctgctgatcgcactgagcctggcactggtgaccaacagcgacatccagctgacccagagcccaagcagcctgagcgcaagcgtgggagaccgagtgaccatcacctgtagcgcaagccaggacatcagcaactacctgaactggtaccagcagaagccaggaaaggcaccaaaggtgctgatctacttcaccagcagcctgcatagcggagtgccaagccgattcagcggaagcggaagcggaaccgacttcaccctgaccatcagcagcctgcagccagaggacttcgcaacctactactgtcagcagtacagcaccgtgccatggaccttcggacagggaaccaaggtggagatcaagcgaaccgtggcagcaccaagcgtgttcatcttcccaccaagcgacgagcagctgaagagcggaaccgcaagcgtggtgtgtctgctgaacaacttctacccacgagaggcaaaggtgcagtggaaggtggacaacgcactgcagagcggaaacagccaggagagcgtgaccgagcaggacagcaaggacagcacctacagcctgagcagcaccctgaccctgagcaaggcagactacgagaagcataaggtgtacgcatgtgaggtgacccatcagggactgagcagcccagtgaccaagagcttcaaccgaggagagtgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 3951 bp, synthetic construct
  misc_feature 1..3951: note = ITR.CB7.CI.aVEGFv4.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5'UTR
  misc_feature 1988..1993: note = kozak
  misc_feature 1993..2052: note = leader
  misc_feature 2053..2421: note = aVEGFv4 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv4 VL
  misc_feature 3211..3531: note = CL
  misc_feature 3532..3537: note = stop cassette
  regulatory 3607..3733: note = polyA_signal - Rabbit globin poly A; regulatory_class = polyA_signal_sequence
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtacagaatgcagctgctgctgctgatcgccctgagcctggccctggtcaccaacagcgaggtccagctggtcgagagcggaggaggactggtccagcccggaggaagcctgagactgagctgcgccgccagcggatacgacttcacccactacggaatgaactgggtcagacaggcccccggaaagggactggagtgggtcggatggatcaacacctacaccggagagcccacctacgccgccgacttcaagagaagattcaccttcagcctggacaccagcaagagcaccgcctacctgcagatgaacagcctgagagccgaggacaccgccgtctactactgcgccaagtacccctactactacggaaccagccactggtacttcgacgtctggggacagggaaccctggtcaccgtcagcagcgccagcaccaagggacccagcgtcttccccctggcccccagcagcaagagcaccagcggaggaaccgccgccctgggatgcctggtcaaggactacttccccgagcccgtcaccgtcagctggaacagcggagccctgaccagcggagtccacaccttccccgccgtcctgcagagcagcggactgtacagcctgagcagcgtcgtcaccgtccccagcagcagcctgggaacccagacctacatctgcaacgtcaaccacaagcccagcaacaccaaggtcgacaagaaggtcgagcccaagagctgcgacaagacccacctgagaaagagaagagcccccgtcaagcagaccctgaacttcgacctgctgaagctggccggagacgtcgagagcaaccccggacccatgtacagaatgcagctgctgctgctgatcgccctgagcctggccctggtcaccaacagcgacatccagctgacccagagccccagcagcctgagcgccagcgtcggagacagagtcaccatcacctgcagcgccagccaggacatcagcaactacctgaactggtaccagcagaagcccggaaaggcccccaaggtcctgatctacttcaccagcagcctgcacagcggagtccccagcagattcagcggaagcggaagcggaaccgacttcaccctgaccatcagcagcctgcagcccgaggacttcgccacctactactgccagcagtacagcaccgtcccctggaccttcggacagggaaccaaggtcgagatcaagagaaccgtcgccgcccccagcgtcttcatcttcccccccagcgacgagcagctgaagagcggaaccgccagcgtcgtctgcctgctgaacaacttctaccccagagaggccaaggtccagtggaaggtcgacaacgccctgcagagcggaaacagccaggagagcgtcaccgagcaggacagcaaggacagcacctacagcctgagcagcaccctgaccctgagcaaggccgactacgagaagcacaaggtctacgcctgcgaggtcacccaccagggactgagcagccccgtcaccaagagcttcaacagaggagagtgctgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 3951 bp, synthetic construct
  misc_feature 1..3951: note = ITR.CB7.CI.aVEGFv5.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5'UTR
  misc_feature 1988..1993: note = kozak
  misc_feature 1993..2052: note = leader
  misc_feature 2053..2421: note = aVEGFv5 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv5 VL
  misc_feature 3211..3531: note = CL
  misc_feature 3532..3537: note = stop cassette
  regulatory 3607..3733: note = polyA_signal - rabbit globin poly A; regulatory_class = polyA_signal_sequence
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccgcatgcagcttcttcttcttatcgctctttctcttgctcttgtgaccaattctgaagtgcagcttgtggaatctggcggcggccttgtgcagccgggcggctctcttcgcctttcttgcgctgcttctggctacgactttacccattacggcatgaattgggtgcgccaggctccgggcaagggccttgaatgggtgggctggatcaatacctacaccggcgaaccgacctacgctgctgactttaagcgccgctttaccttttctcttgacacctctaagtctaccgcttaccttcagatgaattctcttcgcgctgaagacaccgctgtgtactactgcgctaagtacccgtactactacggcacctctcattggtactttgacgtgtggggccagggcacccttgtgaccgtgtcttctgcttctaccaagggcccgtctgtgtttccgcttgctccgtcttctaagtctacctctggcggcaccgctgctcttggctgccttgtgaaggactactttccggaaccggtgaccgtgtcttggaattctggcgctcttacctctggcgtgcatacctttccggctgtgcttcagtcttctggcctttactctctttcttctgtggtgaccgtgccgtcttcttctcttggcacccagacctacatctgcaatgtgaatcataagccgtctaataccaaggtggacaagaaggtggaaccgaagtcttgcgacaagacccatcttcgcaagcgccgcgctccggtgaagcagacccttaattttgaccttcttaagcttgctggcgacgtggaatctaatccgggcccgatgtaccgcatgcagcttcttcttcttatcgctctttctcttgctcttgtgaccaattctgacatccagcttacccagtctccgtcttctctttctgcttctgtgggcgaccgcgtgaccatcacctgctctgcttctcaggacatctctaattaccttaattggtaccagcagaagccgggcaaggctccgaaggtgcttatctactttacctcttctcttcattctggcgtgccgtctcgcttttctggctctggctctggcaccgactttacccttaccatctcttctcttcagccggaagactttgctacctactactgccagcagtactctaccgtgccgtggacctttggccagggcaccaaggtggaaatcaagcgcaccgtggctgctccgtctgtgtttatctttccgccgtctgacgaacagcttaagtctggcaccgcttctgtggtgtgccttcttaataatttttacccgcgcgaagctaaggtgcagtggaaggtggacaatgctcttcagtctggcaattctcaggaatctgtgaccgaacaggactctaaggactctacctactctctttcttctacccttaccctttctaaggctgactacgaaaagcataaggtgtacgcttgcgaagtgacccatcagggcctttcttctccggtgaccaagtcttttaatcgcggcgaatgctgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 37 - DNA, 3951 bp, synthetic construct
  misc_feature 1..3951: note = ITR.CB7.CI.aVEGFv6.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5'UTR
  misc_feature 1993..2052: note = leader
  misc_feature 2053..2421: note = aVEGFv6 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv6 VL
  misc_feature 3211..3531: note = CL
  misc_feature 3532..3537: note = stop cassette
  regulatory 3607..3733: note = polyA_signal - Rabbit globin poly A; regulatory_class = polyA_signal_sequence
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccggatgcagctgctgctgctgatcgccctgagcctggccctggtgaccaacagcgaggtgcagctggtggagagcggaggaggactggtgcagccaggaggaagcctgcggctgagctgcgccgccagcggatacgatttcacccactacggaatgaactgggtgcggcaggccccaggaaagggactggagtgggtgggatggatcaacacctacaccggagagccaacctacgccgccgatttcaagcggcggttcaccttcagcctggataccagcaagagcaccgcctacctgcagatgaacagcctgcgggccgaggataccgccgtgtactactgcgccaagtacccatactactacggaaccagccactggtacttcgatgtgtggggacagggaaccctggtgaccgtgagcagcgccagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtacagcctgagcagcgtggtgaccgtgccaagcagcagcctgggaacccagacctacatctgcaacgtgaaccacaagccaagcaacaccaaggtggataagaaggtggagccaaagagctgcgataagacccacctgcggaagcggcgggccccagtgaagcagaccctgaacttcgatctgctgaagctggccggagatgtggagagcaacccaggaccaatgtaccggatgcagctgctgctgctgatcgccctgagcctggccctggtgaccaacagcgatatccagctgacccagagcccaagcagcctgagcgccagcgtgggagatcgggtgaccatcacctgcagcgccagccaggatatcagcaactacctgaactggtaccagcagaagccaggaaaggccccaaaggtgctgatctacttcaccagcagcctgcacagcggagtgccaagccggttcagcggaagcggaagcggaaccgatttcaccctgaccatcagcagcctgcagccagaggatttcgccacctactactgccagcagtacagcaccgtgccatggaccttcggacagggaaccaaggtggagatcaagcggaccgtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccggggagagtgctgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 3951 bp, synthetic construct; identical to SEQ ID NO: 37
  misc_feature 1..3951: note = ITR.CB7.CI.aVEGFv7.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5'UTR
  misc_feature 1988..1993: note = kozak
  misc_feature 1993..2052: note = leader
  misc_feature 2053..2421: note = aVEGFv7 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv7 VL
  misc_feature 3211..3531: note = CL
  misc_feature 3532..3537: note = stop cassette
  regulatory 3607..3733: note = polyA_signal - rabbit globin poly A; regulatory_class = polyA_signal_sequence
  repeat_region 3822..3951: note = 3'ITR
  source 1..3951: mol_type = other DNA

SEQ ID NO: 39 - DNA, 3951 bp, synthetic construct
  misc_feature 1..3951: note = ITR.CB7.CI.aVEGFv8.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5'UTR
  misc_feature 1988..1993: note = kozak
  misc_feature 1993..2052: note = leader
  misc_feature 2053..2421: note = aVEGFv8 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv8 VL
  misc_feature 3211..3531: note = CL
  misc_feature 3532..3537: note = stop cassette
  regulatory 3607..3733: note = polyA_signal - rabbit globin poly A; regulatory_class = polyA_signal_sequence
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccgcatgcagctgctgctgctgatcgctctgtcactggctctggtgaccaattcagaagtgcagctggtggaatcaggtggtggtctggtgcagcccggtggttcactgcgcctgtcatgcgctgcttcaggttacgactttacccattacggtatgaattgggtgcgccaggctcccggtaagggtctggaatgggtgggttggatcaatacctacaccggtgaacccacctacgctgctgactttaagcgccgctttaccttttcactggacacctcaaagtcaaccgcttacctgcagatgaattcactgcgcgctgaagacaccgctgtgtactactgcgctaagtacccctactactacggtacctcacattggtactttgacgtgtggggtcagggtaccctggtgaccgtgtcatcagcttcaaccaagggtccctcagtgtttcccctggctccctcatcaaagtcaacctcaggtggtaccgctgctctgggttgcctggtgaaggactactttcccgaacccgtgaccgtgtcatggaattcaggtgctctgacctcaggtgtgcatacctttcccgctgtgctgcagtcatcaggtctgtactcactgtcatcagtggtgaccgtgccctcatcatcactgggtacccagacctacatctgcaatgtgaatcataagccctcaaataccaaggtggacaagaaggtggaacccaagtcatgcgacaagacccatctgcgcaagcgccgcgctcccgtgaagcagaccctgaattttgacctgctgaagctggctggtgacgtggaatcaaatcccggtcccatgtaccgcatgcagctgctgctgctgatcgctctgtcactggctctggtgaccaattcagacatccagctgacccagtcaccctcatcactgtcagcttcagtgggtgaccgcgtgaccatcacctgctcagcttcacaggacatctcaaattacctgaattggtaccagcagaagcccggtaaggctcccaaggtgctgatctactttacctcatcactgcattcaggtgtgccctcacgcttttcaggttcaggttcaggtaccgactttaccctgaccatctcatcactgcagcccgaagactttgctacctactactgccagcagtactcaaccgtgccctggacctttggtcagggtaccaaggtggaaatcaagcgcaccgtggctgctccctcagtgtttatctttcccccctcagacgaacagctgaagtcaggtaccgcttcagtggtgtgcctgctgaataatttttacccccgcgaagctaaggtgcagtggaaggtggacaatgctctgcagtcaggtaattcacaggaatcagtgaccgaacaggactcaaaggactcaacctactcactgtcatcaaccctgaccctgtcaaaggctgactacgaaaagcataaggtgtacgcttgcgaagtgacccatcagggtctgtcatcacccgtgaccaagtcatttaatcgcggtgaatgctgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 40 - DNA, 3957 bp, synthetic construct
  misc_feature 1..3957: note = ITR.CB7.CI.aVEGFv9.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1946..1993: note = c-myc 5'UTR
  misc_feature 1994..1999: note = kozak
  misc_feature 1999..2058: note = leader
  misc_feature 2059..2427: note = aVEGFv9 VH
  misc_feature 2428..2748: note = CH1
  misc_feature 2752..2763: note = furin cleavage site
  misc_feature 2764..2835: note = F2A linker
  misc_feature 2836..2895: note = leader
  misc_feature 2896..3216: note = aVEGFv9 VL
  misc_feature 3217..3537: note = CL
  misc_feature 3538..3543: note = Stop Cassette
  regulatory 3613..3739: note = polyA_signal - rabbit globin poly A; regulatory_class = polyA_signal_sequence
  repeat_region 3828..3957: note = 3'ITR
  source 1..3957: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagagctagcgggcactttgcactggaacttacaacacccgagcaaggacgcgactctccaccatgtatagaatgcagcttcttcttcttatcgctcttagccttgctcttgtgacaaacagcgaagtgcagcttgtggaaagcgggggggggcttgtgcagcctggggggagccttagacttagctgtgctgctagcgggtatgattttacacactatgggatgaactgggtgagacaggctcctgggaaggggcttgaatgggtggggtggatcaacacatatacaggggaacctacatatgctgctgattttaagagaagatttacatttagccttgatacaagcaagagcacagcttatcttcagatgaacagccttagagctgaagatacagctgtgtattattgtgctaagtatccttattattatgggacaagccactggtattttgatgtgtgggggcaggggacacttgtgacagtgagcagcgctagcacaaaggggcctagcgtgtttcctcttgctcctagcagcaagagcacaagcggggggacagctgctcttgggtgtcttgtgaaggattattttcctgaacctgtgacagtgagctggaacagcggggctcttacaagcggggtgcacacatttcctgctgtgcttcagagcagcgggctttatagccttagcagcgtggtgacagtgcctagcagcagccttgggacacagacatatatctgtaacgtgaaccacaagcctagcaacacaaaggtggataagaaggtggaacctaagagctgtgataagacacaccttagaaagagaagagctcctgtgaagcagacacttaactttgatcttcttaagcttgctggggatgtggaaagcaaccctgggcctatgtatagaatgcagcttcttcttcttatcgctcttagccttgctcttgtgacaaacagcgatatccagcttacacagagccctagcagccttagcgctagcgtgggggatagagtgacaatcacatgtagcgctagccaggatatcagcaactatcttaactggtatcagcagaagcctgggaaggctcctaaggtgcttatctattttacaagcagccttcacagcggggtgcctagcagatttagcgggagcgggagcgggacagattttacacttacaatcagcagccttcagcctgaagattttgctacatattattgtcagcagtatagcacagtgccttggacatttgggcaggggacaaaggtggaaatcaagagaacagtggctgctcctagcgtgtttatctttcctcctagcgatgaacagcttaagagcgggacagctagcgtggtgtgtcttcttaacaacttttatcctagagaagctaaggtgcagtggaaggtggataacgctcttcagagcgggaacagccaggaaagcgtgacagaacaggatagcaaggatagcacatatagccttagcagcacacttacacttagcaaggctgattatgaaaagcacaaggtgtatgcttgtgaagtgacacaccaggggcttagcagccctgtgacaaagagctttaacagaggggaatgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 41 - DNA, 3951 bp, synthetic construct
  misc_feature 1..3951: note = ITR.CB7.CI.aVEGFv10.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5'UTR
  misc_feature 1988..1993: note = kozak
  misc_feature 1993..2052: note = leader
  misc_feature 2053..2421: note = aVEGFv10 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv10 VL
  misc_feature 3211..3531: note = CL
  misc_feature 3532..3537: note = stop cassette
  regulatory 3607..3733: note = polyA_signal - rabbit globin poly A; regulatory_class = polyA_signal_sequence
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtatcggatgcaactgctgctgctgattgccctgagcctggccctggttaccaacagcgaagttcaactggttgaaagcggcggcggcctggttcaacccggcggcagcctgcggctgagctgtgccgccagcggctatgactttacccattatggcatgaactgggttcggcaagcccccggcaagggcctggaatgggttggctggattaacacctataccggcgaacccacctatgccgccgactttaagcggcggtttacctttagcctggacaccagcaagagcaccgcctatctgcaaatgaacagcctgcgggccgaagacaccgccgtttattattgtgccaagtatccctattattatggcaccagccattggtattttgacgtttggggccaaggcaccctggttaccgttagcagcgccagcaccaagggccccagcgtttttcccctggcccccagcagcaagagcaccagcggcggcaccgccgccctgggctgtctggttaaggactattttcccgaacccgttaccgttagctggaacagcggcgccctgaccagcggcgttcatacctttcccgccgttctgcaaagcagcggcctgtatagcctgagcagcgttgttaccgttcccagcagcagcctgggcacccaaacctatatttgtaacgttaaccataagcccagcaacaccaaggttgacaagaaggttgaacccaagagctgtgacaagacccatctgcggaagcggcgggcccccgttaagcaaaccctgaactttgacctgctgaagctggccggcgacgttgaaagcaaccccggccccatgtatcggatgcaactgctgctgctgattgccctgagcctggccctggttaccaacagcgacattcaactgacccaaagccccagcagcctgagcgccagcgttggcgaccgggttaccattacctgtagcgccagccaagacattagcaactatctgaactggtatcaacaaaagcccggcaaggcccccaaggttctgatttattttaccagcagcctgcatagcggcgttcccagccggtttagcggcagcggcagcggcaccgactttaccctgaccattagcagcctgcaacccgaagactttgccacctattattgtcaacaatatagcaccgttccctggacctttggccaaggcaccaaggttgaaattaagcggaccgttgccgcccccagcgtttttatttttccccccagcgacgaacaactgaagagcggcaccgccagcgttgtttgtctgctgaacaacttttatccccgggaagccaaggttcaatggaaggttgacaacgccctgcaaagcggcaacagccaagaaagcgttaccgaacaagacagcaaggacagcacctatagcctgagcagcaccctgaccctgagcaaggccgactatgaaaagcataaggtttatgcctgtgaagttacccatcaaggcctgagcagccccgttaccaagagctttaaccggggcgaatgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 42 - DNA, 3951 bp, synthetic construct
  misc_feature 1..3951: note = ITR.CB7.CI.aVEGFv11.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5' UTR
  misc_feature 1988..1993: note = kozak
  misc_feature 1993..2052: note = leader
  misc_feature 2053..2421: note = aVEGFv11 VH
  misc_feature 2422..2742: note = CH1
  misc_feature 2746..2754: note = furing cleavage site
  misc_feature 2755..2829: note = F2A linker
  misc_feature 2830..2889: note = leader
  misc_feature 2890..3210: note = aVEGFv11 VL
  misc_feature 3211..3531: note = CL
  misc_feature 3532..3537: note = stop cassette
  regulatory 3607..3733: note = polyA_signal - rabbit globin poly A; regulatory_class = polyA_signal_sequence
  repeat_region 3822..3951: note = 3' ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtatagaatgcagctgctgctgctgatcgctctgtcactggctctggtgaccaactcagaggtgcagctggtggagtcaggaggaggactggtgcagcccggaggatcactgagactgtcatgcgctgcttcaggatatgatttcacccattatggaatgaactgggtgagacaggctcccggaaaaggactggagtgggtgggatggatcaacacctataccggagagcccacctatgctgctgatttcaaaagaagattcaccttctcactggatacctcaaaatcaaccgcttatctgcagatgaactcactgagagctgaggataccgctgtgtattattgcgctaaatatccctattattatggaacctcacattggtatttcgatgtgtggggacagggaaccctggtgaccgtgtcatcagcttcaaccaaaggaccctcagtgttccccctggctccctcatcaaaatcaacctcaggaggaaccgctgctctgggatgcctggtgaaagattatttccccgagcccgtgaccgtgtcatggaactcaggagctctgacctcaggagtgcataccttccccgctgtgctgcagtcatcaggactgtattcactgtcatcagtggtgaccgtgccctcatcatcactgggaacccagacctatatctgcaacgtgaaccataaaccctcaaacaccaaagtggataaaaaagtggagcccaaatcatgcgataaaacccatctgagaaaaagaagagctcccgtgaaacagaccctgaacttcgatctgctgaaactggctggagatgtggagtcaaaccccggacccatgtatagaatgcagctgctgctgctgatcgctctgtcactggctctggtgaccaactcagatatccagctgacccagtcaccctcatcactgtcagcttcagtgggagatagagtgaccatcacctgctcagcttcacaggatatctcaaactatctgaactggtatcagcagaaacccggaaaagctcccaaagtgctgatctatttcacctcatcactgcattcaggagtgccctcaagattctcaggatcaggatcaggaaccgatttcaccctgaccatctcatcactgcagcccgaggatttcgctacctattattgccagcagtattcaaccgtgccctggaccttcggacagggaaccaaagtggagatcaaaagaaccgtggctgctccctcagtgttcatcttccccccctcagatgagcagctgaaatcaggaaccgcttcagtggtgtgcctgctgaacaacttctatcccagagaggctaaagtgcagtggaaagtggataacgctctgcagtcaggaaactcacaggagtcagtgaccgagcaggattcaaaagattcaacctattcactgtcatcaaccctgaccctgtcaaaagctgattatgagaaacataaagtgtatgcttgcgaggtgacccatcagggactgtcatcacccgtgaccaaatcattcaacagaggagagtgctgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 43 - DNA, 3951 bp, synthetic construct
  misc_feature 1..3951: note = ITR.CB7.CI.aVEGFv12.rBG.ITR
  repeat_region 1..130: note = 5'ITR
  regulatory 198..579: note = promoter - CMV IE promoter; regulatory_class = promoter
  regulatory 279..538: note = enhancer - C4 enhancer; regulatory_class = enhancer
  regulatory 582..862: note = promoter - CB promoter; regulatory_class = promoter
  misc_feature 956..1928: note = Intron - chicken beta-actin intron
  5'UTR 1940..1987: note = c-myc 5'UTR
  misc_feature 1988..1993: note = kozak
  misc_feature 1993..2052: note = leader
  misc_feature 2053..2421: note = aVEGFv12 VH
  misc_feature 2422..2742: note = CH1
  misc_feature 2746..2757: note = furing cleavage site
  misc_feature 2758..2829: note = F2A linker
  misc_feature 2830..2889: note = leader
  misc_feature 2890..3210: note = aVEGFv12 VL
  misc_feature 3211..3531: note = CL
  misc_feature 3532..3537: note = stop cassette
  regulatory 3607..3733: note = polyA_signal - rabbit globin poly A; regulatory_class = polyA_signal_sequence
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccgtatgcagctgctgctgctgatagccctgagcctggccctggtgacaaatagcgaggtgcagctggtggagagcgggggggggctggtgcagcccggggggagcctgcgtctgagctgtgccgccagcgggtacgactttacacattacgggatgaattgggtgcgtcaggcccccgggaaagggctggagtgggtggggtggataaatacatacacaggggagcccacatacgccgccgactttaaacgtcgttttacatttagcctggacacaagcaaaagcacagcctacctgcagatgaatagcctgcgtgccgaggacacagccgtgtactactgtgccaaatacccctactactacgggacaagccattggtactttgacgtgtgggggcaggggacactggtgacagtgagcagcgccagcacaaaagggcccagcgtgtttcccctggcccccagcagcaaaagcacaagcggggggacagccgccctggggtgtctggtgaaagactactttcccgagcccgtgacagtgagctggaatagcggggccctgacaagcggggtgcatacatttcccgccgtgctgcagagcagcgggctgtacagcctgagcagcgtggtgacagtgcccagcagcagcctggggacacagacatacatatgtaatgtgaatcataaacccagcaatacaaaagtggacaaaaaagtggagcccaaaagctgtgacaaaacacatctgcgtaaacgtcgtgcccccgtgaaacagacactgaattttgacctgctgaaactggccggggacgtggagagcaatcccgggcccatgtaccgtatgcagctgctgctgctgatagccctgagcctggccctggtgacaaatagcgacatacagctgacacagagccccagcagcctgagcgccagcgtgggggaccgtgtgacaataacatgtagcgccagccaggacataagcaattacctgaattggtaccagcagaaacccgggaaagcccccaaagtgctgatatactttacaagcagcctgcatagcggggtgcccagccgttttagcgggagcgggagcgggacagactttacactgacaataagcagcctgcagcccgaggactttgccacatactactgtcagcagtacagcacagtgccctggacatttgggcaggggacaaaagtggagataaaacgtacagtggccgcccccagcgtgtttatatttccccccagcgacgagcagctgaaaagcgggacagccagcgtggtgtgtctgctgaataatttttacccccgtgaggccaaagtgcagtggaaagtggacaatgccctgcagagcgggaatagccaggagagcgtgacagagcaggacagcaaagacagcacatacagcctgagcagcacactgacactgagcaaagccgactacgagaaacataaagtgtacgcctgtgaggtgacacatcaggggctgagcagccccgtgacaaaaagctttaatcgtggggagtgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 44 - DNA, 3951 bp, synthetic construct
  misc_feature 1..3951: note = ITR.CB7.CI.aVEGFv13.rBG.ITR
  repeat_region 1..130: note = 5'ITR
  misc_feature 131..167: note = part of AAV
  regulatory 198..579: note = promoter - CME IE promoter; regulatory_class = promoter
  misc_feature 204..233: note = promoter start
  regulatory 279..538: note = enhancer - C4 enhancer with 2 mismatches; regulatory_class = enhancer
  misc_feature 561..584: note = CMV promoter end
  regulatory 582..862: note = promoter - CB promoter; regulatory_class = promoter
  misc_feature 585..615: note = begin promoter
  regulatory 836..839: note = TATA box; regulatory_class = TATA_box
  misc_feature 956..1928: note = Intron - chichen beta-actin intron
  misc_feature 1814..1830: note = end of intron
  5'UTR 1940..1987: note = c-myc 5'UTR
  misc_feature 1988..1993: note = kozak
  misc_feature 1993..2052: note = leader
  misc_feature 2053..2421: note = aVEGFv13 VH
  misc_feature 2422..2742: note = CH1
  misc_feature 2746..2757: note = furin cleavage site
  misc_feature 2758..2829: note = F2A linker
  misc_feature 2830..2889: note = leader
  misc_feature 2890..3210: note = aVEGFv13 VL
  misc_feature 3211..3531: note = CL
  misc_feature 3532..3537: note = stop cassette
  regulatory 3607..3733: note = polyA_signal - rabbit globin poly A; regulatory_class = polyA_signal_sequence
  misc_feature 3785..3821: note = part of AAV
  repeat_region 3822..3951: note = 3'ITR
  source 1..3951: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccggatgcagctgctgctgctgatcgccctgagcctggccctggtgaccaacagcgaggtgcagctggtggagagcgggggggggctggtgcagcccggggggagcctgcggctgagctgcgccgccagcgggtacgacttcacccactacgggatgaactgggtgcggcaggcccccgggaaggggctggagtgggtggggtggatcaacacctacaccggggagcccacctacgccgccgacttcaagcggcggttcaccttcagcctggacaccagcaagagcaccgcctacctgcagatgaacagcctgcgggccgaggacaccgccgtgtactactgcgccaagtacccctactactacgggaccagccactggtacttcgacgtgtgggggcaggggaccctggtgaccgtgagcagcgccagcaccaaggggcccagcgtgttccccctggcccccagcagcaagagcaccagcggggggaccgccgccctggggtgcctggtgaaggactacttccccgagcccgtgaccgtgagctggaacagcggggccctgaccagcggggtgcacaccttccccgccgtgctgcagagcagcgggctgtacagcctgagcagcgtggtgaccgtgcccagcagcagcctggggacccagacctacatctgcaacgtgaaccacaagcccagcaacaccaaggtggacaagaaggtggagcccaagagctgcgacaagacccacctgcggaagcggcgggcccccgtgaagcagaccctgaacttcgacctgctgaagctggccggggacgtggagagcaaccccgggcccatgtaccggatgcagctgctgctgctgatcgccctgagcctggccctggtgaccaacagcgacatccagctgacccagagccccagcagcctgagcgccagcgtgggggaccgggtgaccatcacctgcagcgccagccaggacatcagcaactacctgaactggtaccagcagaagcccgggaaggcccccaaggtgctgatctacttcaccagcagcctgcacagcggggtgcccagccggttcagcgggagcgggagcgggaccgacttcaccctgaccatcagcagcctgcagcccgaggacttcgccacctactactgccagcagtacagcaccgtgccctggaccttcgggcaggggaccaaggtggagatcaagcggaccgtggccgcccccagcgtgttcatcttcccccccagcgacgagcagctgaagagcgggaccgccagcgtggtgtgcctgctgaacaacttctacccccgggaggccaaggtgcagtggaaggtggacaacgccctgcagagcgggaacagccaggagagcgtgaccgagcaggacagcaaggacagcacctacagcctgagcagcaccctgaccctgagcaaggccgactacgagaagcacaaggtgtacgcctgcgaggtgacccaccaggggctgagcagccccgtgaccaagagcttcaaccggggggagtgctgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 45 - DNA, 3809 bp, synthetic construct
  misc_feature 1..3809: note = ITR.CMV.PI.aVEGFv7.eMCV.IRES.SV40.ITR
  repeat_region 1..130: note = ITR
  regulatory 191..932: note = promoter - human CMV I.E. enhancer & promoter; regulatory_class = promoter
  misc_feature 1047..1179: note = Intron - Promega chimeric intron
  5'UTR 1248..1295: note = c-myc 5'UTR
  misc_feature 1299..1307: note = kozak
  misc_feature 1305..1364: note = leader
  misc_feature 1365..1685: note = aVEGFv7 VL
  misc_feature 1686..2006: note = CL
  regulatory 2018..2608: note = enhancer - IRES; regulatory_class = enhancer
  misc_feature 2606..2665: note = leader
  misc_feature 2666..3034: note = aVEGFv7 VH
  misc_feature 3035..3355: note = CH1
  regulatory 3384..3615: note = polyA_signal - SV40 late polyadenylation signal; regulatory_class = polyA_signal_sequence
  repeat_region 3680..3809: note = ITR
  source 1..3809: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggggactttgcactggaacttacaacacccgagcaaggacgcgactctagacccaccatgtaccggatgcagctgctgctgctgatcgccctgagcctggccctggtgaccaacagcgatatccagctgacccagagcccaagcagcctgagcgccagcgtgggagatcgggtgaccatcacctgcagcgccagccaggatatcagcaactacctgaactggtaccagcagaagccaggaaaggccccaaaggtgctgatctacttcaccagcagcctgcacagcggagtgccaagccggttcagcggaagcggaagcggaaccgatttcaccctgaccatcagcagcctgcagccagaggatttcgccacctactactgccagcagtacagcaccgtgccatggaccttcggacagggaaccaaggtggagatcaagcggaccgtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccggggagagtgctgataaggccggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgtaccgcatgcagctgctgctgctgatcgccctgagcctggccctggtgaccaacagcgaggtgcagctggtggagagcggaggaggactggtgcagccaggaggaagcctgcggctgagctgcgccgccagcggatacgatttcacccactacggaatgaactgggtgcggcaggccccaggaaagggactggagtgggtgggatggatcaacacctacaccggagagccaacctacgccgccgatttcaagcggcggttcaccttcagcctggataccagcaagagcaccgcctacctgcagatgaacagcctgcgggccgaggataccgccgtgtactactgcgccaagtacccatactactacggaaccagccactggtacttcgatgtgtggggacagggaaccctggtgaccgtgagcagcgccagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtacagcctgagcagcgtggtgaccgtgccaagcagcagcctgggaacccagacctacatctgcaacgtgaaccacaagccaagcaacaccaaggtggataagaaggtggagccaaagagctgcgataagacccacctgaagtgatgaaagcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 46 - DNA, 3686 bp, synthetic construct
  misc_feature 1..3686: note = ITR.CMV.PI.aVEGFv7.fmdIRES.SV40.ITR
  repeat_region 1..130: note = ITR
  regulatory 191..932: note = promoter - human CMV I.E. enhancer and promoter; regulatory_class = promoter
  regulatory 897..901: note = TATA box; regulatory_class = TATA_box
  misc_feature 1047..1179: note = Intron - Promega chimeric intron
  5'UTR 1248..1295: note = c-myc 5'UTR
  misc_feature 1299..1307: note = kozak
  misc_feature 1305..1313: note = leader
  misc_feature 1314..1364: note = leader
  misc_feature 1365..1685: note = aVEGFv7 VL
  misc_feature 1686..2006: note = CL
  misc_feature 2021..2482: note = FMDV
  misc_feature 2501..2542: note = leader
  misc_feature 2543..2911: note = aVEGFv7 VH
  misc_feature 2912..3232: note = CH1
  regulatory 3261..3492: note = polyA_signal - Sv40 late polyadenylation signal; regulatory_class = polyA_signal_sequence
  repeat_region 3557..3686: note = ITR
  source 1..3686: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggggactttgcactggaacttacaacacccgagcaaggacgcgactctagacccaccatgtaccggatgcagctgctgctgctgatcgccctgagcctggccctggtgaccaacagcgatatccagctgacccagagcccaagcagcctgagcgccagcgtgggagatcgggtgaccatcacctgcagcgccagccaggatatcagcaactacctgaactggtaccagcagaagccaggaaaggccccaaaggtgctgatctacttcaccagcagcctgcacagcggagtgccaagccggttcagcggaagcggaagcggaaccgatttcaccctgaccatcagcagcctgcagccagaggatttcgccacctactactgccagcagtacagcaccgtgccatggaccttcggacagggaaccaaggtggagatcaagcggaccgtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccggggagagtgctgataaggccggccagcaggtttccccaactgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgggacactcgggatctgagaaggggactggggcttctataaaagcgctcggtttaaaaagcttctatgcctgaataggtgaccggaggtcggcacctttcctttacaattaatgaccctaatgtaccgcatgcagctgctgctgctgatcgccctgagcctggccctggtgaccaacagcgaggtgcagctggtggagagcggaggaggactggtgcagccaggaggaagcctgcggctgagctgcgccgccagcggatacgatttcacccactacggaatgaactgggtgcggcaggccccaggaaagggactggagtgggtgggatggatcaacacctacaccggagagccaacctacgccgccgatttcaagcggcggttcaccttcagcctggataccagcaagagcaccgcctacctgcagatgaacagcctgcgggccgaggataccgccgtgtactactgcgccaagtacccatactactacggaaccagccactggtacttcgatgtgtggggacagggaaccctggtgaccgtgagcagcgccagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtacagcctgagcagcgtggtgaccgtgccaagcagcagcctgggaacccagacctacatctgcaacgtgaaccacaagccaagcaacaccaaggtggataagaaggtggagccaaagagctgcgataagacccacctgaagtgatgaaagcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 47 - DNA, 3619 bp, synthetic construct
  misc_feature 1..3619: note = ITR.CMV.PI.aVEGFv7.cMycIRES.SV40.ITR
  repeat_region 1..130: note = ITR
  regulatory 191..932: note = promoter - human CMV I.E. enhancer and promoter; regulatory_class = promoter
  regulatory 897..901: note = TATA box; regulatory_class = TATA_box
  misc_feature 1047..1179: note = Intron - Promega chimeric intron
  5'UTR 1248..1295: note = c-myc 5'UTR
  misc_feature 1299..1307: note = kozak
  misc_feature 1305..1313: note = leader
  misc_feature 1314..1364: note = leader
  misc_feature 1365..1685: note = aVEGFv7 VL
  misc_feature 1686..2006: note = CL
  misc_feature 2021..2415: note = IRES c-myc
  misc_feature 2226..2273: note = mini c-myc IRES
  misc_feature 2275: note = this C to T mutaion increases expression
  misc_feature 2434..2475: note = leader
  misc_feature 2476..2844: note = aVEGFv7 VH
  misc_feature 2845..3165: note = CH1
  regulatory 3194..3425: note = polyA_signal - SV40 late polyadenylation signal; regulatory_class = polyA_signal_sequence
  repeat_region 3490..3619: note = ITR
  source 1..3619: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggggactttgcactggaacttacaacacccgagcaaggacgcgactctagacccaccatgtaccggatgcagctgctgctgctgatcgccctgagcctggccctggtgaccaacagcgatatccagctgacccagagcccaagcagcctgagcgccagcgtgggagatcgggtgaccatcacctgcagcgccagccaggatatcagcaactacctgaactggtaccagcagaagccaggaaaggccccaaaggtgctgatctacttcaccagcagcctgcacagcggagtgccaagccggttcagcggaagcggaagcggaaccgatttcaccctgaccatcagcagcctgcagccagaggatttcgccacctactactgccagcagtacagcaccgtgccatggaccttcggacagggaaccaaggtggagatcaagcggaccgtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccggggagagtgctgataaggccggccaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgtaccgcatgcagctgctgctgctgatcgccctgagcctggccctggtgaccaacagcgaggtgcagctggtggagagcggaggaggactggtgcagccaggaggaagcctgcggctgagctgcgccgccagcggatacgatttcacccactacggaatgaactgggtgcggcaggccccaggaaagggactggagtgggtgggatggatcaacacctacaccggagagccaacctacgccgccgatttcaagcggcggttcaccttcagcctggataccagcaagagcaccgcctacctgcagatgaacagcctgcgggccgaggataccgccgtgtactactgcgccaagtacccatactactacggaaccagccactggtacttcgatgtgtggggacagggaaccctggtgaccgtgagcagcgccagcaccaagggaccaagcgtgttcccactggccccaagcagcaagagcaccagcggaggaaccgccgccctgggatgcctggtgaaggattacttcccagagccagtgaccgtgagctggaacagcggagccctgaccagcggagtgcacaccttcccagccgtgctgcagagcagcggactgtacagcctgagcagcgtggtgaccgtgccaagcagcagcctgggaacccagacctacatctgcaacgtgaaccacaagccaagcaacaccaaggtggataagaaggtggagccaaagagctgcgataagacccacctgaagtgatgaaagcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 48 - protein, 738 aa, unidentified
  REGION 1..738: note = AAV8 capsid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - DNA, 2217 bp, unidentified
  misc_feature 1..2217: note = Nucleic acid sequence of AAV8 capsid
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

(19) United States
(12) Patent Application Publication
(10) Pub. No.: US 2026/0224753 A1
(43) Pub. Date: Aug. 6, 2026

(54) COMPOSITIONS FOR TREATMENT OF WET AGE-RELATED MACULAR DEGENERATION

(71) Applicant: The Trustees of the University of Pennsylvania, Philadelphia, PA (US)

(72) Inventors: Anna P. Tretiakova, Philadelphia, PA (US); James M. Wilson, Philadelphia, PA (US)

(73) Assignee: The Trustees of the University of Pennsylvania, Philadelphia, PA (US)

(21) Appl. No.: 19/423,915

(22) Filed: Dec. 17, 2025

Related U.S. Application Data
(63) Continuation of application No. 17/340,752, filed on Jun. 7, 2021, now abandoned.
Application No. 17/340,752 is a continuation of application No. 16/093,420, filed on Apr. 14, 2017, now Pat. No. 11,197,937.
(60) Provisional application No. 62/323,184, filed on Apr. 15, 2016.
(60) Provisional application No. 62/331,100, filed on May 3, 2016.
(60) Provisional application No. 62/442,946, filed on Jan. 5, 2017.
(60) Provisional application No. 62/460,515, filed on Feb. 17, 2017.
(60) Provisional application No. 62/466,721, filed on Mar. 3, 2017.

(51) Int. Cl. A61K 48/00; A61K 9/00; A61K 9/10; A61K 35/761; C07K 16/22; C12N 15/86

(52) U.S. Cl. CPC A61K 48/0075; A61K 9/0019; A61K 9/0048; A61K 9/10; A61K 35/761; A61K 48/005; C07K 16/22; C12N 15/86; C07K 2317/55; C07K 2319/33; C12N 2750/14143

(57) ABSTRACT

A recombinant adeno-associated virus (rAAV) having an AAV8 capsid which is suitable for intra-retinal injection is provided herein. The rAAV comprises a vector genome packaged within the capsid which contains, operably linked to regulatory elements which direct expression of anti-human vascular endothelial growth factor (VEGF) antigen binding antibody fragment (aVEGF), a coding sequence for aVEGF, wherein the coding sequence is operably linked to regulatory elements which direct expression of the anti-VEGF Fab in the eye. Also provided herein are liquid suspensions containing these rAAV8.aVEGF and methods of using same for treatment of wet AMD and other ocular conditions.

### CROSS-REFERENCE TO RELATED APPLICATIONS

This is a continuation of U.S. patent application Ser. No. 17/340,752, filed Jun. 7, 2021, which claims benefit of U.S. patent application Ser. No. 16/093,420, filed Oct. 12, 2018, which is a National Stage Entry under 35 U.S.C. 371 of International Patent Application No. PCT/US2017/027529, filed Apr. 14, 2017, which claims the benefit of U.S. Provisional Patent Application No. 62/323,184, filed Apr. 15, 2016, U.S. Provisional Patent Application No. 62/331,100, filed May 3, 2016, U.S. Provisional Patent Application No. 62/442,946, filed Jan. 5, 2017, U.S. Provisional Patent Application No. 62/460,515, filed Feb. 17, 2017, and U.S. Provisional Patent Application No. 62/466,721, filed Mar. 3, 2017. These applications are incorporated by reference herein.

### REFERENCE TO AN ELECTRONIC SEQUENCE LISTING

The electronic sequence listing filed herewith named “UPN-16-7683USC2” with size of 286,394 bytes, created on date of Dec. 17, 2025, and the contents of the electronic sequence listing (e.g., the sequences and text therein) are incorporated herein by reference in entirety.

### BACKGROUND OF THE INVENTION

Age-related macular degeneration (AMD) is a progressive degenerative macular disease attacking the region of highest visual acuity (VA), the macula, and is the leading cause of blindness in Americans 60 years or older (NIH Medline Plus (2008), Leading cause of blindness, NIH Medline Plus 3 (2) 14-15. www.nlm.nih.gov/medlineplus/magazine/issues/summer08/articles/summer08pg14-15.html). The neovascular “wet” form of the disease (nAMD or wet AMD) is characterized by choroidal neovascularization which is marked by proliferation of blood vessels and cells including those of the retinal pigment epithelium (RPE) (Carmeliet (2005) Nature 438:932-936). Ultimately, photoreceptor death and scar formation result in a severe loss of central vision and the inability to read, write, and recognize faces or drive. Many patients can no longer maintain gainful employment, carry out daily activities and consequently report a diminished quality of life (Mitchell and Bradley (2006), Health Qual Life Outcomes 4:97). Preventative therapies have demonstrated little effect and therapeutic strategies have focused primarily on treating the neovascular lesion.

Some currently available treatments for wet AMD include laser photocoagulation, photodynamic therapy with verteporfin, and intravitreal (IVT) injections with the vascular endothelial growth factor (VEGF) inhibitors such as pegaptanib, ranibizumab, bevacizumab or aflibercept (Schmidt-Erfurth, (2014) Guidelines for the management of neovascular age-related macular degeneration by the European Society of Retina Specialists (EURETINA) Br J Ophthalmol 98:1144-1167). While these therapies have some effect on best-corrected visual acuity (BCVA), their effects may be limited in restoring visual acuity and in duration (Schmidt-Erfurth, cited above, 2014, AAO PPP (2015) Preferred Practice Patterns: Age Related Macular Degeneration. American Academy of Ophthalmology).

Several drugs in market that are used to treat wet AMD rely on a mechanism that inhibits VEGF and must be injected intravitreally. While these treatments are reported to succeed in prohibiting the disease from progressing, they require frequent injections of the drug.

Of specific note, ranibizumab, a recombinant, humanized monoclonal IgG1 antigen-binding fragment (Fab) is designed to bind and inhibit all active forms of human (VEGF). Ranibizumab is a humanized monoclonal antibody fragment produced in Escherichia coli cells by recombinant DNA technology. The binding of ranibizumab to VEGF-A prevents the interaction of VEGF-A with its receptors VEGFR-1 and VEGFR-2 on the surface of endothelial cells. This binding inhibits endothelial cell proliferation and neovascularization, as well as vascular leakage, all of which are thought to contribute to the progression of the neovascular (wet) form of age-related macular degeneration (Wet AMD). The safety and efficacy of ranibizumab (Lucentis®) has been established, and ranibizumab is United States (US) Food & Drug Administration (FDA) approved for IVT injection treatment in patients with neovascular AMD, as well as other retinal diseases (initially approved FDA 2006). While long term therapy with either monthly ranibizumab or monthly/every 8 week aflibercept may slow the progression of vision loss and improve vision, none of these treatments prevent neovascularization from recurring (Brown et al (2006) N Engl J Med, 355:1432-44; Rosenfeld et al., (2006) N Engl J Med 355:1419-31; Schmidt-Erfurth, 2014, cited above). Each has to be re-administered to prevent the disease from worsening. The need for repeat treatments can incur additional risk to patients and is inconvenient for both patients and treating physicians.

### SUMMARY OF THE INVENTION

In one aspect, the invention provides a recombinant adeno-associated virus (rAAV) having an AAV8 capsid which is suitable for sub-retinal and/or intra-retinal injection. The AAV8 capsid packages a vector genome that provides for the production of a soluble antigen-binding fragment (Fab) of a human monoclonal antibody (MAb) that binds and inhibits human vascular endothelial growth factor (hVEGF)—the expression product is sometimes referred to herein as “anti-h VEGF Fab” or “aVEGF”.

The vector genome packaged within the rAAV8 capsid, comprises: (a) an AAV inverted terminal repeat(s) (ITR(s)) flanking an expression construct for the anti-VEGF Fab; (b) the expression construct having regulatory elements comprising a chicken beta-actin promoter or a ubiquitin C promoter that direct expression in the eye of a transgene encoding anti-hVEGF Fab; and (c) the transgene which encodes the heavy and light chains of the anti-hVEGF Fab, each chain having a heterologous leader sequence added to its amino terminus, and wherein the coding sequences for the heavy and light chains are separated by a coding sequence for a “cleavable” peptide linker or an IRES (internal ribosome entry site) to ensure production of separate heavy and light chain polypeptides, and a polyadenylation signal. The resulting transgene expression products may contain an amino acid residue in addition to those normally found in Fab heavy chains.

In particular embodiments, codon sequences for the heavy and light chains optimized for expression in human cells are used. As illustrated by the examples, these can include but are not limited to AAV2/8.CB7.CI.aVEGFv1.rBG; AAV2/8.CB7.CI.aVEGFv2.rBG; AAV2/8.CB7.CI.aVEGFv3.rBG; AAV2/8.CB7.CI.aVEGFv4.rBG; AAV2/8.CB7.CI.aVEGFv5.rBG; AAV2/8.CB7.CI.aVEGFv6.rBG; AAV2/8.CB7.CI.aVEGFv7.rBG; AAV2/8.CB7.CI.aVEGFv8.rBG; AAV2/8.CB7.CI.VEGFv9.rBG; AAV2/8.CB7.CI.aVEGFv10.rBG; AAV2/8.CB7.CI.aVEGFv11.rBG; AAV2/8.CB7.CI.aVEGFv12.rBG; AAV2/8.CB7.CI.aVEGFv13.rBG.

As used herein, “AAV2/8” and “AAV8” are used interchangeably to refer to a recombinant AAV having an AAV8 capsid and vector genome flanked by AAV2 ITRs.

In yet another aspect, a liquid suspension of any of the foregoing rAAV8.aVEGF for sub-retinal and/or intra-retinal injection is provided. The composition comprises an aqueous liquid and rAAV8.aVEGF as described herein, and optionally one or more excipients, preservatives, and/or surfactants.

In still a further aspect, a method for delivering an anti-hVEGF Fab to a patient having wet age-related related macular degeneration is provided. The method involves subretinally injecting the patient's eye with the liquid suspension comprising the rAAV8 vector carrying the expression construct for the anti-h VEGF Fab.

In certain embodiments, the invention provides a rAAV as described herein, or a liquid suspension, administrable subretinally to a patient. In certain embodiments, use of a rAAV or a liquid suspension, for subretinal administration to a patient is provided. The patient may have been previously diagnosed with wet age-related macular degeneration, or another ocular condition as defined herein.

In still a further embodiment, a product comprising: (a) a first container comprising an rAAV8.anti-hVEGF Fab and an aqueous liquid, (b) optionally a second container comprising a diluent, and (c) a needle for injection. In certain embodiments, the product is an injection kit.

The invention is illustrated by the examples below which demonstrate that subretinal administration of an rAAV8.aVEGF vector results in gene transfer throughout the retina, and expression of anti-VEGF Fab throughout the retina and in the vitreous and anterior chamber fluids. This result is surprising in view of prior art gene therapy studies that demonstrated that gene transfer spreads laterally outside of the original injection bleb but remains confined to those expanded boundaries and did not achieve gene transfer and transgene expression outside this expanded area of injection (the “bleb” formed in the retina at the injection site); and offers an advantage over standard of care treatment for nAMD in that a single administration of the rAAV8.aVEGF vector should result in (i) continuous delivery of the effective amounts of the VEGF inhibitor throughout the retina which may in turn improve performance as compared to repeated IVT administrations of high dose boluses of the VEGF inhibitor that dissipate over time; and (ii) avoidance of repeated ocular injections which pose additional risks and inconvenience to patients. Each aspect may improve therapeutic outcome.

Still other aspects and advantages of the invention will be apparent from the following detailed description of the invention.

### BRIEF DESCRIPTION OF THE FIGURES

FIG. 1 provides a schematic representation of an AAV8 vector genome containing a gene cassette flanked by the AAV2 inverted terminal repeat (ITRs) and expressing human anti-vascular endothelial growth factor (anti-VEGF) antigen binding antibody fragment (Fab). Control elements include the CB7 promoter consisting of the chicken β-actin promoter and CMV enhancer, chicken β-actin and a rabbit β-globin poly A signal. The nucleic acid sequences coding for the heavy and light chains of anti-VEGF Fab are separated by a self-cleaving furin (F)/F2A linker. A furin recognition site that consists of arginine-lysine-arginine-arginine amino acid sequence was used. In addition, the light and heavy chain each contain a heterologous leader peptide which directs nascent peptide into appropriate cellular compartment where leader peptide is processed away from the mature protein by the host cellular machinery. These and the other synthetic anti-VEGF constructs are termed herein, AAV.aVEGF.

FIG. 2 provides the expression levels and kinetics of AAV8.CB7.aVEGFv1 or rAAV8.UbC.aVEGFv1 at various time points post-injection of the left eye (os) or right eye (od). AAV8.CB7.aVEGF-Rv1 is the top line with the closed circle. AAV8.UBC.aVEGF-Rv1 is the bottom line with the closed circle. The middle line with the open circles is AAV8.UBC.aVEGF-Rv1.

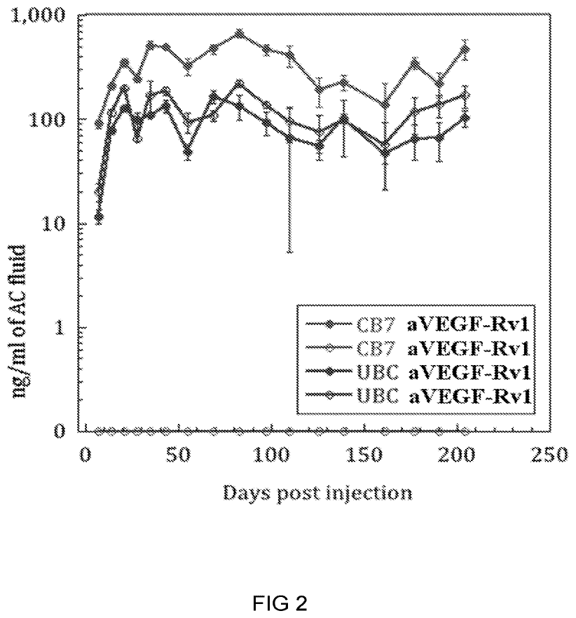

FIGS. 3A-3D show expression of anti-VEGF Fab in anterior chamber fluid and blood for animals in Groups 2 and 3 as described in Example 3 in which Cynomolgus monkeys were administered a single dose of 1.00×10¹¹ GC/eye of AAV2/8 vectors into each eye subretinally. Anterior chamber fluid and blood were collected at prespecified timepoints. Expression of the anti-VEGF Fab was determined using enzyme-linked immunosorbent assay. In FIGS. 3A and 3B, the results for animals in Group 2. In FIGS. 3C and 3D, the results for Group 3 are presented. In FIGS. 3B and 3D, the gray area in the panel presenting the results in serum denotes baseline levels. Circles denote females and squares denote males. Samples were analyzed in duplicate. The results are presented as mean±standard deviation. Abbreviations: Fab=fragment antigen-binding; GC=genome copies; OD=right eye; OS=left eye; VEGF=vascular endothelial growth factor.

FIGS. 4A-4D show expression of anti-VEGF Fab in anterior chamber fluid and blood for animals in Groups 5 and 6 as described in Example 3 in which cynomolgus monkeys were administered a single dose of 1.00×10¹¹ GC/eye of AAV2/8 vectors into each eye subretinally. Anterior chamber fluid and blood were collected at prespecified timepoints. Expression of the anti-VEGF Fab was determined using enzyme-linked immunosorbent assay. The results for Group 5 are presented in FIGS. 4A and 4B and the results for Group 6 is presented in FIGS. 4C and 4D. In FIGS. 4B and 4D, the gray area in the panel presenting the results in serum denotes baseline levels. Circles denote females and squares denote males. Samples were analyzed in duplicate. The results are presented as mean±standard deviation. Abbreviations: Fab=fragment antigen-binding; GC=genome copies; OD=right eye; OS=left eye; VEGF=vascular endothelial growth factor.

FIGS. 5A-5D provide levels of mRNA for AAV8.aVEGF test vector in retina determined by RT-qPCR. Cynomolgus monkeys were administered a single dose of 1.00×10¹² GC/eye of a AAV8.aVEGF test vector or FFB-314 into the right eye subretinally. Levels of mRNA for the AAV8.aVEGF test vector were determined in different portions of the dissected retinas by quantitative reverse transcription polymerase chain reaction (RT-qPCR). In left panels, schematics of injection sites are depicted. In middle panels, retinal dissections are presented. In right panels, levels of mRNA for AAV8.aVEGF test vector mRNA (GC per 100 ng of RNA) in 4 sections of retina are depicted. Abbreviations: BV=major blood vessel; F=fovea; GC=genome copies; IB=injection bleb; ID=identification; O=optic disk; UD=undetected.

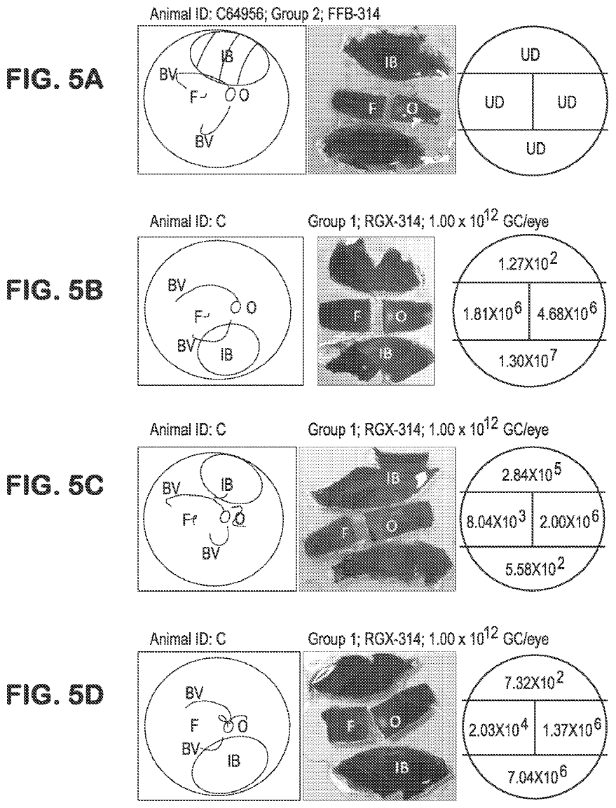

FIGS. 6A-6D provide results of expression of anti-VEGF Fab in anterior chamber fluid, vitreous, and retina (Group 2, Example 6). Cynomolgus monkeys were administered a single dose of 1.00× 10¹¹ GC/eye of AAV2/8 vector subretinally. These data represent results from different AAV8.aVEGF vectors than shown in FIGS. 5A-5D. Concentrations of anti-VEGF Fab were determined in anterior chamber fluid, vitreous, and 4 different parts of retina. Eyes were dissected as described in FIGS. 5A-5D. In FIGS. 6A and 6C, infrared spectral domain optical coherence tomography images of the retinas with boundaries of injection site are depicted. In FIGS. 6B and 6D, graphs of concentrations of anti-VEGF Fab are presented. In this figure, the results for animals in Group 2 in Example 6 are presented. Abbreviations: ACF=anterior chamber fluid; BV=major blood vessel; F=fovea; Fab=fragment antigen-binding; FOV=middle section containing fovea; GC=genome copies; IB=injection bleb; ID=identification; INF=inferior retinal section; O=optic disk; ODI=middle section containing optic disk; SUP=superior retinal section; VEGF=vascular endothelial growth factor; VIT=vitreous.

FIGS. 7A-7D provide results of expression of anti-VEGF Fab in anterior chamber fluid, vitreous, and retina (Group 3, Example 6). Cynomolgus monkeys were administered a single dose of 1.00×10¹¹ GC/eye of AAV2/8 vector subretinally. These data represent results from different AAV8.aVEGF vectors than shown in FIGS. 5A-5D. Concentrations of anti-VEGF Fab were determined in anterior chamber fluid, vitreous, and 4 different parts of retina. Eyes were dissected as described in FIGS. 5A-5D. In FIGS. 7A and 7C, infrared spectral domain optical coherence tomography images of the retinas with boundaries of injection site are depicted. In the graphs of FIGS. 7B and 7D, concentrations of anti-VEGF Fab are presented. In this figure, the results for animals in Group 3 in Example 6 are presented. Abbreviations: ACF=anterior chamber fluid; BV=major blood vessel; F=fovea; Fab=fragment antigen-binding; FOV-middle section containing fovea; GC=genome copies; IB=injection bleb; ID=identification; INF=inferior retinal section; O=optic disk; ODI=middle section containing optic disk; SUP=superior retinal section; VEGF=vascular endothelial growth factor; VIT=vitreous.

FIGS. 8A-8D provide results of expression of anti-VEGF Fab in anterior chamber fluid, vitreous, and retina (Group 5, Example 6). Cynomolgus monkeys were administered a single dose of 1.00×10¹¹ GC/eye of AAV2/8 vector subretinally. These data represent results from different AAV8.aVEGF vectors than shown in FIGS. 5A-5D. Concentrations of anti-VEGF Fab were determined in anterior chamber fluid, vitreous, and 4 different parts of retina. Eyes were dissected as described in FIGS. 5A-5D. In FIGS. 8A and 8C, infrared spectral domain optical coherence tomography images of the retinas with boundaries of injection site are depicted. In the graphs of FIGS. 8B and 8D, concentrations of anti-VEGF Fab are presented. In this figure, the results for animals in Group 5 in Example 6 are presented. Abbreviations: ACF=anterior chamber fluid; BV=major blood vessel; F=fovea; Fab=fragment antigen-binding; FOV-middle section containing fovea; GC=genome copies; IB=injection bleb; ID=identification; INF=inferior retinal section; O=optic disk; ODI=middle section containing optic disk; SUP=superior retinal section; VEGF=vascular endothelial growth factor; VIT=vitreous.

FIG. 9 provides a flow diagram of the manufacturing process.

FIGS. 10A-10D illustrate the results of an rcAAV assay for AAV8. wtAAV8 is spiked into different GC amounts of AAV vector and the cap gene copy number per 1 μg of 293 cell DNA is determined after three successive passages of the cell lysate onto fresh cells. 3 different spike levels of wtAAV8 [one level per panel: 1×10² GC, 1×10³ GC and 1×10⁴ GC] 4 different vector amounts [0 GC (dark square), 1×10⁹ GC (gray square) 1×10¹⁰ GC (triangle) and 1×10¹¹ GC (marked with X)] are shown and background levels are indicated (controls).

### DETAILED DESCRIPTION OF THE INVENTION

Recombinant, replication-defective adeno-associated virus (rAAV) vectors having an AAV8 capsid and compositions containing same which are suitable for subretinal injections to deliver an anti-VEFG antibody binding fragment (Fab). Also provided are compositions containing same, and in particularly, liquid aqueous suspension. Uses of these compositions are also provided.

The rAAV8 vectors are designed to express an anti-VEGF antibody binding fragment (Fab) in mammalian, and more particularly, human cells. These anti-VEGF Fabs are particularly well suited for treatment of age-related macular degeneration (AMD). For convenience, these vectors are terms rAAV8.AMD. As described herein, a series of novel AAV8.aVEGF constructs have been developed which have demonstrated high yield, expression levels, and/or activity.

The invention is illustrated by the examples below which demonstrate that subretinal administration of an rAAV8.aVEGF vector results in gene transfer throughout the retina, and expression of anti-VEGF Fab throughout the retina and in the vitreous and anterior chamber fluids. This result is surprising in view of prior art gene therapy studies that demonstrated that gene transfer spreads laterally outside of the original injection bleb but remains confined to those expanded boundaries and did not achieve gene transfer and transgene expression outside this expanded area of injection (the “bleb” formed in the retina at the injection site); and offers an advantage over standard of care treatment for nAMD in that a single administration of the rAAV8.aVEGF vector should result in (i) continuous delivery of the effective amounts of the VEGF inhibitor throughout the retina which may in turn improve performance as compared to repeated IVT administrations of high dose boluses of the VEGF inhibitor that dissipate over time; and (ii) avoidance of repeated ocular injections which pose additional risks and inconvenience to patients. Each aspect may improve therapeutic outcome.

The present invention provides constructs encoding a novel anti-VEGF Fab having, at a minimum, a heavy chain amino acid sequence of SEQ ID NO: 1 and a light chain amino acid sequence of SEQ ID NO:2, each of which has been engineered to have an exogenous leader sequence for each the heavy chain and light chain. In certain constructs illustrated herein, the leader sequence is derived from a human IL2 leader. Further, in certain constructs illustrated in the working examples, the heavy and light chains are separated by a furin/F2a linker, which may result may result in one or more extra amino acids being added to the heavy chain [SEQ ID NO:1]. In one embodiment, a single arginine [R] is added to the heavy chain. However, in certain embodiments, another linker may be selected and/or a different system may result in no additional amino acid, or one or more extra amino acids [e.g., R, Lys (K), RK, RKR, RKRR among others]. In prior provisional applications, the resulting constructs were termed herein, aVEGF-R. However, for clarity, these constructs encoding the anti-VEGF Fab transgene product described herein, are referred to as: anti-VEGF Fab, aVEGF, anti-hVEGF, anti-human VEGF, or anti-VEGF Fab transgene product. In the constructs encoding this transgene product, a numerical designation following the term aVEGF, e.g., aVEGFv1, aVEGFv2, aVEGFv3, through aVEGFv13, refers to different nucleic acid coding sequences for the open reading frame of the immunoglobulin heavy chain and light chains.

In certain embodiments, the amino acid sequence of the anti-VEGF Fab has 513 amino acids, including anti-VEGF is heavy and light chain separated by extra amino acids as a result of the linker. For example, while each of the following expression cassettes encodes the same anti-VEGF heavy chain and light chain, in one embodiment, there may be one amino acid added to the last position of the heavy chain. In still other embodiments, there may be two, three, four or more extra amino acids attached to the heavy chain. For example, in certain embodiments, the nucleic acid sequences coding for the heavy and light chains of anti-VEGF Fab are separated by a self-cleaving furin (F)/F2A linker. A furin recognition site that consists of arginine-lysine-arginine-arginine amino acid sequence may be used. Due to the mechanism of furin-mediated cleavage, vector-expressed anti-VEGF Fab may contain an additional arginine (R) residue added to the last position of the heavy chain [SEQ ID NO: 1]. In other embodiments, the vector-expressed anti-VEGF Fab may contain the dipeptide arginine-lysine at the end of the heavy chain, the tripeptide arginine-lysine-arginine at the end of the heavy chain, or the polypeptide arginine-lysine-arginine-arginine at the end of the heavy chain. In certain embodiments, the vector expressed anti-VEGF Fab are a heterogeneous mixture of two or more of these Fab products. Other furin cleavage sites can be used (arginine-X-X-arginine, or arginine-X-lysine or arginine-arginine), which can also generate C-terminal heterogeneity. In other words, other vector expressed anti-VEGF Fabs may be a heterogeneous population of the Fab in which the heavy chain has 0, 1, 2, 3, or 4 amino acids at its C-terminus as a result of the linker processing. In addition, the light and heavy chain each contain a heterologous leader peptide which directs nascent peptide into appropriate cellular compartment where leader peptide is processed away from the mature protein by the host cellular machinery. In certain embodiments, the anti-VEGF Fab contains no HC or LC leader sequences. See, e.g., SEQ ID NO: 33.

In certain embodiments, the anti-VEGF Fab heavy chain has the amino acid sequence of residues 21-252 of SEQ ID NO: 33 with a leader sequence. In other embodiments, the anti-VEGF Fab light chain has the amino acid sequence of residues 300-513 of SEQ ID NO: 33 with a leader sequence. For example, the leader sequence may be from about 15 to about 25 amino acids, preferably about 20 amino acids. In some embodiments, the leader has the sequence of amino acids 1-20 of SEQ ID NO: 33.

In one embodiment, the coding sequences for the heavy chain and light chain of anti-VEGFv1 are provided in SEQ ID NO: 24. More particularly, the heavy chain variable region open reading frame (ORF) is provided in nucleotides (nt) 1843 to 2211 and the heavy chain constant region (CH1) ORF is provided in nt 2212-2532, with reference to SEQ ID NO: 24. Thus, the aVEGFv1 heavy chain, without the leader, has the nucleic acid sequence of nt 1843 to 2532. The light chain variable region (VL) ORF is provided in nt 2680 to 3000 and the light chain constant region (CL) is provided in nt 3001 to 3321 of SEQ ID NO: 24. Thus, the aVEGFv2 light chain, without the leader, has the nucleic acid sequence of nt 2680 to 3321 of SEQ ID NO: 24.

In another embodiment, the coding sequences for the heavy chain and light chain of anti-VEGFv2 are provided in SEQ ID NO: 3. More particularly, the VH ORF is provided in nt 2059 to 2427 and the CHI is provided in 2428 to 2748 of SEQ ID NO: 3; the heavy chain without the leader has the nucleic acid sequence of nt 2059 to 2748 of SEQ ID NO: 3. The VL ORF is provided in nt 2896 to 3216 and CL is provided in nt 3217 to 3536 of SEQ ID NO: 3; the light chain without the leader sequence has the nucleic acid sequence of nt 2896 to 3536 of SEQ ID NO: 3.

In yet another embodiment, the coding sequences for the heavy chain and light chain of aVEGFv3 are provided in SEQ ID NO: 19. The VH ORF is provided in nt 1842 to 2210 and the CHI is provided in nt 2211 to 2531 of SEQ ID NO: 19; the heavy chain without the leader has the nucleic acid sequence of nt 1842 to 2531 of SEQ ID NO: 19. The VL ORF is provided in nt 2679 to 2999 and CL is provided in nt 3000 to 3320 of SEQ ID NO: 19; the light chain without the leader has the nucleic acid sequence of nt 2670 to 3320 of SEQ ID NO: 19.

In a further embodiment, the coding sequences for the heavy and light chain of aVEGFv4 are provided in SEQ ID NO: 35. The heavy chain leader sequence is encoded by nt 1993-2052, the VH ORF is at nt 2053-2421 and the CH1 is at nt 2422-2742 of SEQ ID NO: 35. As in the other constructs described herein, as a result of the location of the F2A cleavage site, sequences encoding additional amino acids may be retained on the VH chain. The light chain leader sequence is encoded by nt 2830-2889; the VL ORF is provided in nt 2890-3210; the CL ORF is located at nt 3211-3531 of SEQ ID NO: 35.

In a further embodiment, the coding sequences for the heavy and light chain of aVEGFv5 is provided within SEQ ID NO: 36. The heavy chain leader sequence is encoded by nt 1993-2052, the VH ORF is encoded by nt 2053-2421, and the CH1 is encoded by nt 2422-2742 of SEQ ID NO: 36. As in the other constructs described herein, as a result of the location of the F2A cleavage site, sequences encoding additional amino acids may be retained on the VH chain. The light chain leader sequence is encoded by nt 2830-2889; the VL ORF is provided in nt 2890-3210; the CL ORF is located at nt 3211-3531 of SEQ ID NO: 36.

In a further embodiment, the coding sequences for the heavy and light chain of aVEGFv6 is provided within SEQ ID NO: 37. The heavy chain leader sequence is encoded by nt 1993-2051, the VH ORF is encoded by nt 2053-2421, and the CH1 is encoded by nt 2422-2742 of SEQ ID NO: 37. As in the other constructs described herein, as a result of the location of the F2A cleavage site, sequences encoding additional amino acids may be retained on the VH chain. The light chain leader sequence is encoded by nt 2830-2889; the VL ORF is provided in nt 2890-3210; the CL ORF is located at nt 3211-3531 of SEQ ID NO: 37.

In a further embodiment, the coding sequences for the heavy and light chain of aVEGFv7 is provided within SEQ ID NO: 38. The heavy chain leader sequence is encoded by nt 1993-2052, the VH ORF is encoded by nt 2053-2421, and the CH1 is encoded by nt 2422-2742 of SEQ ID NO: 38. As in the other constructs described herein, as a result of the location of the F2A cleavage site, an additional Arg codon is retained on the VH chain. The light chain leader sequence is encoded by nt 2830-2889; the VL ORF is provided in nt 2890-3210; the CL ORF is located at nt 3211-3531 of SEQ ID NO: 38.

In a further embodiment, the coding sequences for the heavy and light chain of aVEGFv8 is provided within SEQ ID NO: 39. The heavy chain leader sequence is encoded by nt 1993-2052, the VH ORF is encoded by nt 205-2421, and the CH1 is encoded by nt 2422-2742 of SEQ ID NO: 39. As in the other constructs described herein, as a result of the location of the F2A cleavage site, an additional Arg codon is retained on the VH chain. The light chain leader sequence is encoded by nt 2830-2889; the VL ORF is provided in 2890-3210; the CL ORF is located at nt 3211-3531 of SEQ ID NO: 39.

In a further embodiment, the coding sequences for the heavy and light chain of aVEGFv9 is provided within SEQ ID NO: 40. The heavy chain leader sequence is encoded by nt 1999-2058, the VH ORF is encoded by nt 2059-2427, and the CH1 is encoded by nt 2428-2748 of SEQ ID NO: 40. As in the other constructs described herein, as a result of the location of the F2A cleavage site, an additional Arg codon is retained on the VH chain. The light chain leader sequence is encoded by nt 2836-2895; the VL ORF is provided in nt 2896-3216; the CL ORF is located at nt 3217-3637 of SEQ ID NO: 40.

In a further embodiment, the coding sequences for the heavy and light chain of aVEGFv10 is provided within SEQ ID NO: 41. The heavy chain leader sequence is encoded by nt 1993-2052, the VH ORF is encoded by nt 2053-2421, and the CH1 is encoded by nt 2422-2742 of SEQ ID NO: 41. As in the other constructs described herein, as a result of the location of the F2A cleavage site, an additional Arg codon is retained on the VH chain. The light chain leader sequence is encoded by nt 2830-2889; the VL ORF is provided in nt 2890-3210; the CL ORF is located at nt 3211-3231 of SEQ ID NO: 41.

In a further embodiment, the coding sequences for the heavy and light chain of aVEGFv11 is provided within SEQ ID NO: 42. The heavy chain leader sequence is encoded by nt 1993-2052, the VH ORF is encoded by nt 2053-2421, and the CH1 is encoded by nt nt 2422-2742 of SEQ ID NO: 42. As in the other constructs described herein, an F2A cleavage site is located between the end of the heavy chain and the beginning of the light chain. The light chain leader sequence is encoded by nt 2830-2889; the VL ORF is provided in nt 2890-3210; the CL ORF is located at nt 3211-3531 of SEQ ID NO: 42.

In a further embodiment, the coding sequences for the heavy and light chain of aVEGFv12 is provided within SEQ ID NO: 43. The heavy chain leader sequence is encoded by nt 1993-2052, the VH ORF is encoded by nt 2053-2421, and the CH1 is encoded by nt 2422-2742 of SEQ ID NO: 43. The light chain leader sequence is encoded by nt 2830-2889; the VL ORF is provided in nt 2890-3210; the CL ORF is located at nt 3211-3531 of SEQ ID NO: 43.

In a further embodiment, the coding sequences for the heavy and light chain of aVEGFv13 is provided within SEQ ID NO: 44. The heavy chain leader sequence is encoded by nt 1993-2052, the VH ORF is encoded by nt 2053-2421, and the CH1 is encoded by nt 2422-2742 of SEQ ID NO: 44. The light chain leader sequence is encoded by nt 2830-2889; the VL ORF is nt 2890-3210; the CL ORF is located at nt 3211-3531 of SEQ ID NO: 44.

Ranibizumab is described herein as a positive control and is currently marketed under the brand name Lucentis®. It is described as a Fab moiety of a high affinity version of recombinant humanized monoclonal antibody rhuMAb vascular endothelial growth factor (VEGF). It consists of a 214-residue light chain linked by a disulfide bond at its C-terminus to the 231-residue N-terminal segment of the heavy chain. The expected amino acid sequences of the heavy and light chains are provided in SEQ ID NO: 1 and 2. CAS number 347396-82-1.

As used herein, an “immunoglobulin domain” refers to a domain of an antibody heavy chain or light chain as defined with reference to a conventional, full-length antibody. More particularly, a full-length antibody contains a heavy (H) chain polypeptide which contains four domains: one N-terminal variable (VH) region and three C-terminal constant (CH1, CH2 and CH3) regions and a light (L) chain polypeptide which contains two domains: one N-terminal variable (VL) region and one C-terminal constant (CL) region. An Fc region may contain two domains (CH2-CH3). A Fab region contains one constant and one variable domain for each the heavy and light chains.

In one embodiment, rAAV.aVEGF vector has an AAV8 capsid and a vector genome packaged therein which comprises at least one element heterologous to AAV8. In one embodiment, the vector genome contains, from 5′ to 3′: (a) an AAV 5′ ITR; (b) an enhancer; (c) a promoter; (d) an intron; (e) a leader sequence and the anti-VEGF heavy chain coding sequence; (f) a furin-F2a linker; (g) a leader sequence and the anti-VEFG light chain coding sequence; (h) a polyA signal; and (i) an AAV3′ ITR.

In certain embodiments, the processing of anti-VEGF Fab heavy chain and light chains is directed by leader peptides that are derived from human IL2 protein. In one embodiment the leader sequence is an interleukin (IL) IL-2 leader sequence, which may be the wild-type human IL2, MYRMQLLSCIALSLALVINS [SEQ ID NO: 29], or a mutated leader, such as MYRMQLLLLIALSLALVTNS [SEQ ID NO: 30] or MRMQLLLLIALSLALVINS [SEQ ID NO: 31]. In another embodiment, a human serpinF1 secretion signal may be used as leader peptides. Other leader sequences can be used, or other leaders exogenous to the heavy and light chain.

As used in the following description of the vector genome unless otherwise specified as the light chain or heavy chain, reference to a coding sequence (e.g., aVEGFv2) encompasses the anti-VEGF heavy chain-furin/F2a linker-anti-VEGF light chain. In one embodiment, a nucleic acid sequence encoding the furin recognition site Arginine-Lysine-Arginine-Arginine is selected. In certain embodiments, nucleic acids encoding a F2A linker which is a 24 amino acid peptide derived from FMDV (GenBank #CAA2436.1) is selected. However, if desired, an IRES sequence, e.g., such as derived from encephalomycarditis virus (EMCV): SEQ ID NO: 32: [TATGCTAGTACGTCTCTCAAGGATAAGTAAGTAATATTAAGGTACGGGAGGTAT TGGACAGGCCGCAATAAAATATCTTTATTTTCATTACATCTGTGTGTTGGTTTTTT GTGTGAATCGATAGTACTAACATACGCTCTCCATCAAAACAAAACGAAACAAAA CAAACTAGCAAAATAGGCTGTCCCCAGTGCAAGTGCAGGTGCCAGAACATTTCT CTGGCCTAACTGGCCGGTACCTGAGCTCTAGTTTCACTTTCCCTAGTTTCACTTTC CCTAGTTTCACTTTCCCTAGTTTCACTTTCCCTAGTTTCACTTTCCCCTCGAGGAT ATCAAGATCTGGCCTCGGCGGCCAG], cMyc [Nanbru C, et al (1997). J. Biol. Chem. 272, 32061-32066; Stoneley M, et al., (1998). Oncogene 16, 423-428.], or foot and mouth disease (FMD) may be selected.

Inverted terminal repeats (ITR) from AAV2 may be selected. Vectors having ITRs from a different source than its capsid are termed “pseudotyped”. In certain embodiments, ITRs from a source other than AAV2 may be selected for this construct to generate another pseudotyped AAV. Alternatively, ITRs from the same source as the capsid may be selected. In certain embodiments, ITRs may be selected to generate a self-complementary AAV, such as defined infra.

In certain embodiments, the promoter is CB7, a hybrid between a cytomegalovirus (CMV) immediate early enhancer (C4) and the chicken beta actin promoter. In other embodiments, the promoter is a ubiquitin C (UbC) promoter. See, e.g., WO 2001/091800. See, e.g., GenBank R accession numbers AF232305 (rat) and D63791 (human), respectively. Still other promoters and/or enhancers may be selected. See, e.g., cytomegalovirus (CMV) immediate early enhancer (260 bp, C4; GenBank #K03104.1). Chicken beta-actin promoter (281 bp; CB; GenBank #X00182.1). In still other embodiments, multiple enhancers and/or promoters may be included.

In certain embodiments, an intron is included. One suitable intron is a chicken beta-actin intron. In one embodiment, the intron is 875 bp (GenBank #X00182.1). In another embodiment, a chimeric intron available from Promega is used. However, other suitable introns may be selected.

The vector genomes described herein include a polyadenylation signal (polyA). A variety of suitable polyA are known. In one example, the polyA is rabbit beta globin, such as the 127 bp rabbit beta-globin polyadenylation signal (GenBank #V00882.1). In other embodiments, an SV40 polyA signal is selected. Still other suitable polyA sequences may be selected.

Optionally, other suitable vector elements may be selected which may include, e.g., a UTR sequence or a Kozak sequence.

In one embodiment, the vector genome contains, ITR-CB7-CI-aVEGFv2-rBG-ITR, [SEQ ID NO: 3]. In another embodiment, the vector genome contains: ITR-UbC-CI-aVEGFv2-SV40-ITR. [SEQ ID NO: 9]. In one embodiment, the vector genome contains, ITR-CB7-CI-aVEGFv3-rBG-ITR [SEQ ID NO: 14]. In another embodiment, the vector genome contains: ITR-UbC-PI-aVEGFv3-SV40-ITR [SEQ ID NO: 19]. In another embodiment, the vector genome contains: ITR-UbC-PI-aVEGFv1-SV40-ITR [SEQ ID NO: 24]. In a further embodiment, the vector genome contains AAV2-ITR-CB7.CI.aVEGFv4.rBG-AAV2 ITR [SEQ ID NO: 35]. In a further embodiment, the vector genome contains AAV2-ITR-CB7.CI.aVEGFv5.rBG-AAV2 ITR [SEQ ID NO: 36]. In a further embodiment, the vector genome contains AAV2-ITR-CB7.Cl.aVEGFv6.rBG-AAV2 ITR [SEQ ID NO: 37]. In a further embodiment, the vector genome contains AAV2-ITR-CB7.CI.aVEGFv7.rBG-AAV2 ITR [SEQ ID NO: 38]. In a further embodiment, the vector genome contains AAV2-ITR-CB7.CI.aVEGFv8.rBG-AAV2 ITR [SEQ ID NO: 39]. In a further embodiment, the vector genome contains AAV2-ITR-CB7.CI.aVEGFv9.rBG-AAV2 ITR [SEQ ID NO: 40]. In a further embodiment, the vector genome contains AAV2-ITR-CB7.CI.aVEGFv10.rBG-AAV2 ITR [SEQ ID NO: 41]. In a further embodiment, the vector genome contains AAV2-ITR-CB7.CI.aVEGFv11.rBG-AAV2 ITR [SEQ ID NO: 42]. In a further embodiment, the vector genome contains AAV2-ITR-CB7.CI.aVEGFv12.rBG-AAV2 ITR [SEQ ID NO: 43]. In a further embodiment, the vector genome contains AAV2 ITR-CB7.CI.aVEGFv13.rBG-AAV2 ITR [see, SEQ ID NO: 44]. In a further embodiment, the vector genome contains AAV2 ITR-CMV.PI.aVEGFv7.eCMVIres.aVEGF.SV40-AAV2 ITR [SEQ ID NO: 45]. In another embodiment, the vector genome contains AAV2 ITR.CMV.PI.aVEGF.FMDVIIRES.SV40-ITR [SEQ ID NO: 46]. In still a further embodiment, the vector genome contains AAV2 ITR.CMV.PI.aVEGF.cMycIRES.Fab.SV40-ITR [SEQ ID NO: 47].

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in prokaryotic cells, mammalian cells, or both. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99:119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production of a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

As used herein, “AAV8 capsid” refers to the AAV8 capsid having the amino acid sequence of GenBank accession: YP_077180 (SEQ ID NO: 48) encoded by nucleic acid sequence of NCBI Reference Sequence: NC_006261.1 (SEQ ID NO: 49), both of which are incorporated by reference herein. Some variation from this encoded sequence is encompassed by the present invention, which may include sequences having about 99% identity to the referenced amino acid sequence in GenBank accession: YP_077180; U.S. Pat. Nos. 7,282,199, 7,790,449; 8,319,480; 8,962,330; 8,962,332, (i.e., less than about 1% variation from the referenced sequence). In another embodiment, the AAV8 capsid may have the VP1 sequence of the AAV8 variant described in WO2014/124282, which is incorporated by reference herein. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003), US 2013/0045186A1, and WO 2014/124282. In certain embodiments, an AAV8 variant which shows tropism for the desired target cell, e.g., photoreceptors, RPE or other ocular cells is selected. For example, an AAV8 capsid may have Y447F, Y733F and T494V mutations (also called “AAV8 (C&G+T494V)” and “rep2-cap8 (Y447F+733F+T494V)”), as described by Kay et al, Targeting Photoreceptors via Intravitreal Delivery Using Novel, Capsid-Mutated AAV Vectors, PLOS One. 2013; 8 (4): e62097. Published online 2013 Apr. 26, which is incorporated herein by reference. See, e.g., Mowat et al, Tyrosine capsid-mutant AAV vectors for gene delivery to the canine retina from a subretinal or intravitreal approach, Gene Therapy 21, 96-105 (January 2014), which is incorporated herein by reference. In another embodiment, the AAV capsid is an AAV8 capsid, which preferentially targets bipolar cells. See, WO 2014/024282, which is incorporated herein by reference.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 32 amino acids, about 330 amino acids, or a peptide fragment thereof or the corresponding nucleic acid sequence coding sequencers. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999). As used herein, the term “operably linked” refers to both expression control sequences that are contiguous with the gene of interest and expression control sequences that act in trans or at a distance to control the gene of interest.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

The term “exogenous” when used with reference to a protein or nucleic acid sequences indicates two or more sequences or subsequences which are from different sources, e.g., an AAV and a human protein.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

rAAV8.aVEGF Formulation

The rAAV8.aVEGF formulation is a suspension containing an effective amount of rAAV8.aVEGF vector suspended in an aqueous solution. In certain embodiments, the suspension contains buffered saline, optionally with a surfactant and/or other excipients. A buffered saline typically contains a physiologically compatible salt or mixture of salts, e.g. phosphate buffered saline, sodium chloride, or a mixture thereof.

In one embodiment, the formulation may contain, e.g., about 1×10⁸ GC/eye to about 7×10¹² GC/eye, or about 5×10⁹ GC/eye to about 1×10¹¹ GC/eye, or about 10¹⁰ GC/eye, or about as measured by oqPCR or digital droplet PCR (ddPCR) as described in, e.g., M. Lock et al, Hu Gene Therapy Methods, Hum Gene Ther Methods. 2014 April; 25 (2): 115-25. doi: 10.1089/hgtb.2013.131. Epub 2014 Feb. 14, which is incorporated herein by reference.

For example, a suspension as provided herein may contain both NaCl and KCl. The pH may be in the range of 6.5 to 8, or 7.2 to 7.6. pH may be assessed using any suitable method, e.g., USP <791> [US Pharmacopeial Convention, reference standards]. A suitable surfactant, or combination of surfactants, may be selected from among a Poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension. In one embodiment, the rAAV8.aVEGF formulation is a suspension containing at least 1×10¹¹ genome copies (GC)/mL, or greater, e.g., about 1×10¹³ GC/mL as measured by oqPCR or digital droplet PCR (ddPCR) as described in, e.g., M. Lock et al, Hu Gene Therapy Methods, Hum Gene Ther Methods. 2014 April; 25 (2): 115-25. doi: 10.1089/hgtb.2013.131. Epub 2014 Feb. 14, which is incorporated herein by reference. In one embodiment, the vector is suspended in an aqueous solution containing 180 mM sodium chloride, 10 mM sodium phosphate, 0.001% Poloxamer 188, pH 7.3. The formulation is suitable for use in human subjects and is administered subretinally.

In order to ensure that empty capsids are removed from the dose of AAV8.aVEGF that is administered to patients, empty capsids are separated from vector particles during the vector purification process. In one embodiment, the vector particles containing packaged genomes are purified from empty capsids using the process described in International Patent Application No. PCT/US16/65976, filed Dec. 9, 2016 and its priority documents, US Patent Appln Nos. 62/322,098, filed Apr. 13, 2016 and 62/266,341, filed on Dec. 11, 2015, and entitled “Scalable Purification Method for AAV8”, which is incorporated by reference herein. Briefly, a two-step purification scheme is described which selectively captures and isolates the genome-containing rAAV vector particles from the clarified, concentrated supernatant of a rAAV production cell culture. The process utilizes an affinity capture method performed at a high salt concentration followed by an anion exchange resin method performed at high pH to provide rAAV vector particles which are substantially free of rAAV intermediates.

In one embodiment, the pH used is from 10 to 10.4 (about 10.2) and the rAAV particles are at least about 50% to about 90% purified from AAV8 intermediates, or a pH of 10.2 and about 90% to about 99% purified from AAV8 intermediates. In one embodiment, this is determined by genome copies. A stock or preparation of rAAV8 particles (packaged genomes) is “substantially free” of AAV empty capsids (and other intermediates) when the rAAV8 particles in the stock are at least about 75% to about 100%, at least about 80%, at least about 85%, at least about 90%, at least about 95%, or at least 99% of the rAAV8 in the stock and “empty capsids” are less than about 1%, less than about 5%, less than about 10%, less than about 15% of the rAAV8 in the stock or preparation. In one embodiment, the formulation is be characterized by an rAAV stock having a ratio of “empty” to “full” of 1 or less, preferably less than 0.75, more preferably, 0.5, preferably less than 0.3.

In a further embodiment, the average yield of rAAV particles is at least about 70%. This may be calculated by determining titer (genome copies) in the mixture loaded onto the column and the amount presence in the final elutions. Further, these may be determined based on q-PCR analysis and/or SDS-PAGE techniques such as those described herein or those which have been described in the art.

For example, to calculate empty and full particle content, VP3 band volumes for a selected sample (e.g., an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the BI anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacylamide gels (e.g., Novex). Silver staining may be performed using SilverXpress (Invitrogen, CA) according to the manufacturer's instructions. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is provided herein which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25 (2): 115-25. doi: 10.1089/hgtb.2013.131. Epub 2014 Feb. 14.

### Manufacturing

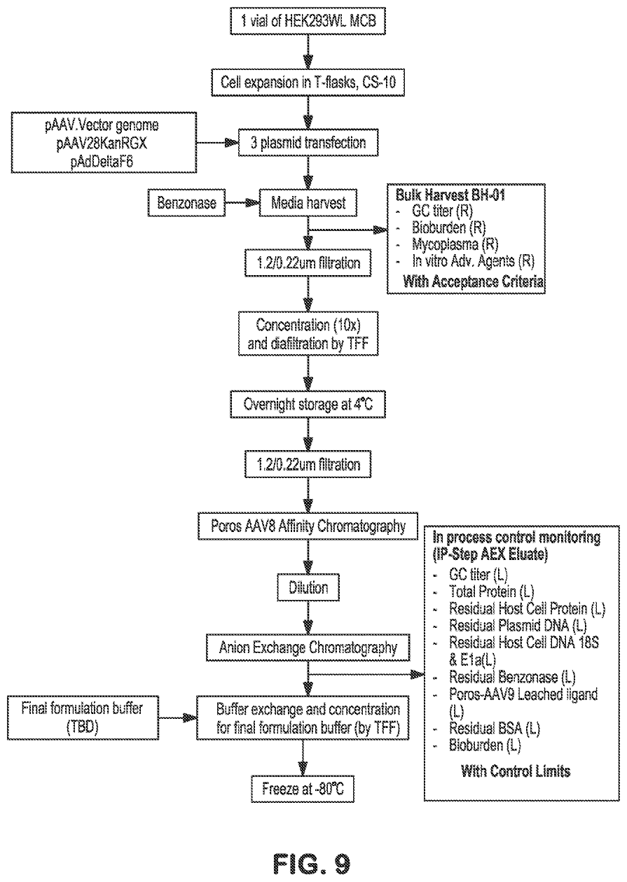

The rAAV8.aVEGF vector can be manufactured as shown in the flow diagram shown in FIG. 9. Briefly, cells (e.g. HEK 293 cells) are propagated in a suitable cell culture system and transfected for vector generation. The rAAV8.aVEGF vector can then be harvested, concentrated and purified to prepare bulk vector which is then filled and finished in a downstream process. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest.

The crude cell harvest may thereafter be subject method steps such as concentration of the vector harvest, diafiltration of the vector harvest, microfluidization of the vector harvest, nuclease digestion of the vector harvest, filtration of microfluidized intermediate, purification by chromatography, purification by ultracentrifugation, buffer exchange by tangential flow filtration, and formulation and filtration to prepare bulk vector.

In a specific embodiment, the methods used for manufacturing the gene therapy vectors are described in the examples herein.

### Patient Population

Patients who are candidates for treatment include those with neovascular age-related macular degeneration, macular edema following retinal vein occlusion (RVO), diabetic macular edema (DME), diabetic retinopathy (non-proliferative diabetic retinopathy (NPDR), proliferative diabetic retinopathy (PDR) in patients with DME, diabetic retinopathy in patients with diabetic macular edema. These patients are particularly well suited for subretinal treatment with an AAV8.aVEGF composition as described herein.

Patients who are candidates for intraocular, including, e.g., subretinal and/or intravitreal administration, with an AAV8.aVEGF as described herein include those with macular degeneration, neovascular/wet/exudative age-related macular degeneration, macular edema following retinal vein occlusion (RVO) (including central retinal vein occlusion (CRVO) and branch retinal vein occlusion (BRVO)) central/hemi/branch retinal vein occlusion, retinal artery occlusion; retinal neovascularization; diabetic macular edema (DME), diabetic retinopathy (non-proliferative diabetic retinopathy (NPDR), proliferative diabetic retinopathy (PDR)) in patients with DME, diabetic retinopathy without macular edema (including pre-treatment of vitrectomy for proliferative diabetic retinopathy); active photocoagulated diabetic retinopathy; choroidal neovascularization, rare causes of choroidal neovascularization (angioid streaks, choroiditis [including choroiditis secondary to ocular histoplasmosis], idiopathic degenerative myopia, retinal dystrophies, rubeosis iridis, and trauma), idiopathic choroidal neovascularization, corneal neovascularization; retinopathy of prematurity, optic nerve head perfusion, retrolental fibroplasia; retinal degeneration; vitreomacular traction syndrome; retinal detachment, diabetic traction retinal detachment, submacular vascularized pigment epithelial detachments, Vogt Koyanagi Harada Disease, pigment epithelial detachment, pigment epithelium rip; vitreoretinopathy proliferative; vitreoretinal surgery in diabetic tractional retinal detachment, polypoidal choroidal vasculopathy; punctate inner choroidopathy (PIC); multifocal choroiditis; central serous chorioretinopathy (CSC), serpiginous choroiditis, vitreous hemorrhage, pars plana vitrectomy for vitreous hemorrhage, diabetic premacular hemorrhage with active fibrovascular proliferation; Choroidal hemorrhage amblyopia; myopia, myopic choroidal neovascularization, choroidal subfoveal/juxtafoveal neovascularization in high myopia; choroidal melanoma; ocular histoplasmosis syndrome, tecalcitrant inflammatory ocular neovascularization (neovascularization, tuberculosis, multifocal serpiginous choroiditis, harada toxoplasmosis); Pseudoxanthoma elasticum; hereditary eye diseases; corneal endothelial cell loss; Vogt Koyanagi Harada Disease, non-arteritic anterior ischemic optic neuropathy; cystoid macular edema; refractory cystoid macular oedema; idiopathic macular telangiectasia; Coat's disease (Coates' disease, also known as exudative retinitis or retinal telangiectasis); glaucoma, neovascular glaucoma, steroid-induced glaucoma, ocular hypertension, Glaucoma surgery; control of wound healing; uveal melanoma; uveitis; radiation maculopathy, pattern dystrophy, radiation retinopathy, radiation necrosis; Hippel-Lindau Disease; Von Hippel-Lindau Syndrome; endophthalmitis; neuromyelitis optica spectrum disorder; pterygium, primary pterygium (including as adjunctive therapy for primary pterygium surgery), recurrent pterygium; retinal drusen; eye neoplasms; intraocular melanoma; cataract; corneal graft failure; trabeculectomy; lipid keratopathy, penetrating keratoplasty, herpetic keratopathy, rosacea; retinal angioma; retinovascular disease; vision disorders, vitreoretinopathy proliferative; iris neovascularization (NV); corneal NV, including pannus, pars planitis sarcoid or Eale's disease.

Patients who are candidates for treatment with an AAV8.aVEGF (the anti-VEGF transgene product) in a regimen which involves a combination with, but not limited to 24GyE proton, 16GyE, Xylocaine, Proparacaine Hydrochloride, Tetravisc, Acuvail, Zimura, Triamcinolone acetonide, Ranibizumab, or Ozurdex. Examples of suitable indications include those in the preceding paragraph. For example, a combination regimen involving an AAV8.aVEGF with one or more of the drugs listed above, may be used for treatment of exudative age-related macular degeneration, central retinal vein occlusion, idiopathic polypoidal choroidal vasculopathy, and/or diabetic macular edema.

The AAV8.aVEGF composition described herein are also useful in preventing vascularization in a number of cancers, neoplasms and other diseases associated with VEGF. Such compositions may be administered for any suitable route, including, e.g., intravenous, intralesional, direct delivery to a tumor or organ, among others. Such patients may include those with Acute Lymphoblastic Leukemia (ALL), Acute Myeloid Leukemia (AML), Adrenocortical Carcinoma, Adrenocortical Carcinoma, AIDS-Related Cancers, Kaposi Sarcoma, AIDS-Related Lymphoma, Primary CNS Lymphoma, Anal Cancer, Appendix Cancer, Astrocytomas, Atypical Teratoid/Rhabdoid Tumor, Basal Cell Carcinoma of the Skin, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Non-Hodgkin Lymphoma, Carcinoid Tumors, Carcinoma of Unknown Primary, Cardiac Tumors, Central Nervous System Atypical Teratoid/Rhabdoid Tumor, Embryonal Tumors, Germ Cell Tumor, Primary CNS Lymphoma, Cervical Cancer, Unusual Cancers of Childhood, Cholangiocarcinoma, Bile Duct Cancer, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Central Nervous System Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Intraocular Melanoma, Retinoblastoma, Fallopian Tube Cancer, Fibrous Histiocytoma of Bone, Malignant, Osteosarcoma, Gallbladder Cancer, Gastric Cancer, Childhood Gastric Cancer, Gastrointestinal Stromal Tumors (GIST), Germ Cell Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular Cancer, Histiocytosis, Langerhans Cell, Hodgkin Lymphoma, Hypopharyngeal Cancer, Intraocular Melanoma, Islet Cell Tumors, Pancreatic Neuroendocrine Tumors, Kaposi Sarcoma, Kidney Cancer, Langerhans Cell Histiocytosis, Laryngeal Cancer, Childhood Laryngeal Cancer and Papillomatosis, Leukemia, Lip and Oral Cavity Cancer, Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Intraocular Melanoma, Merkel Cell Carcinoma, Malignant Mesothelioma, Metastatic Cancer, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasms, Mycosis Fungoides, Myelodysplastic Syndromes, Myelodysplastic, Myeloproliferative Neoplasms, Myelogenous Leukemia, Chronic (CML), Acute Myeloid Leukemia (AML), Chronic Myeloproliferative Neoplasms, Nasal Cavity and Paranasal Sinus Cancer; Nasopharyngeal Cancer, Neuroblastoma, Non-Hodgkin Lymphoma, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Pharyngeal Cancer, Pheochromocytoma, Plasma Cell Neoplasm/Multiple Myeloma, Pleuropulmonary Blastoma; Pregnancy and Breast Cancer, Primary Central Nervous System (CNS) Lymphoma, Primary Peritoneal Cancer, Prostate Cancer, Rectal Cancer, Recurrent Cancer, Renal Cell Cancer, Retinoblastoma, Salivary Gland Cancer, Sarcoma, Childhood Rhabdomyosarcoma, Childhood Vascular Tumors, Ewing Sarcoma, Osteosarcoma, Uterine Sarcoma, Sézary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Metastatic, Stomach Cancer, Cutaneous T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter (Kidney (Renal Cell) Cancer), Carcinoma of Unknown Primary, Childhood Cancer of Unknown Primary, Unusual Cancers of Childhood, Ureter and Renal Pelvis, Urethral Cancer; Endometrial Uterine Cancer, Uterine Sarcoma, Uterine Leiomyosarcomas, Vaginal Cancer, Vascular Tumors, Vulvar Cancer, Wilms Tumor and Other Childhood Kidney Tumors, abdominal neoplasms (adenocarcinoma; hepatocellular, papillary serous mullerian, Lapatinib, colorectal, ovarian, fallopian tube, peritoneal ncer/neoplasms/carcinoma/tumors); lymphoproliferative disorder; small intestine cancer; acoustic neuroma (e.g. vestibular Schwannoma, Neurofibromatosis Type 2); acute myeloid leukemia, acute respiratory distress syndrome (ARDS), head and neck cancer; squamous cell carcinoma, multiple myeloma, non-hodgkin's lymphoma; B-cell lymphoma, sarcoma, neuroblastoma, advanced cancer, alignant neoplasms of female genital organs; metastatic or unresectable solid tumor, anaplastic astrocytoma, Colon Cancer, Metastatic Melanoma, Malignant Ascites, Renal Cell Carcinoma, Glioblastoma, Gliosarcoma, Colorectal Liver Metastases, Advanced Malignancy, Myeloma, Gestational Trophoblastic Neoplasia, Choriocarcinoma, Placental Site Trophoblastic Tumor, Epithelioid Trophoblastic Tumor, Biliary Tract Cancer, Malignant Glioma, Cervical Cancer, Uterine Cancer, Mesothelioma. Candidates thereof are treated with said composition alone or in combination with anti-cancer treatments, for example but not limited to paclitaxel, carboplatin, oxaliplatin, radiation, capecitabine, irinotecan, fluorouracil, doxorubin hydrochloride liposome, erlotinib hydrochloride, irinotecan hydrochloride, Irinotecan hydrochloride hydrate (CPT-11), gemcitabine hydrochloride, Pazopanib Hydrochloride, topotecan hydrochloride, Trifluridine/tipiracil hydrochloride, Pegylated Liposomal Doxorubicin Hydrochloride, enzastaurin hydrochloride, mitoxantrone hydrochloride; epirubicin hydrochloride, docetaxel, gemcitabine, erlotinib, cisplatin, chemotherapy, cetuximab, FOLFIRI-Cetuximab, 5-Fluorouracil (5-FU), LV5FU2, cyclophosphamide, temozolomide, pemetrexed, levofolinate calcium (1-LV), Leucovorin Calcium, FOLFOX, FOLFOX6, mFOLFOX, FOLFOXIRI, FOLFIRI, doxorubicin, Liposomal Doxorubicin, doxorubicin HCL liposome, Sorafenib Tosylate, sorafenib, triamcinolone, Triamcinolone acetonide, trastuzumab, everolimus, sunitinib, dexamethasone, conventional surgery, xeloda, radiotherapy, temsirolimus, pazopanib, Leucovorin (LV), 1-LV, anitumumab, epirubicin, verteporfin, AMG 655, Amgen 386, AMG 479, AMG 706, AMG 951, AMG 102, Folinic Acid, levo-folinic acid, etoposide, BAY 43-9006, atezolizumab, Interferon Alfa-2b, Interferon alpha-2a, interferon alfa, Gamma-Interferon-1b, Photodynamic Therapy, vinorelbine tartrate, vinorelbine, topotecan, tarceva, pemetrexed disodium, estramustine phosphate sodium, Imetelstat sodium, XELOX, RAD001, pegfilgrastim, paclitaxel albumin-stabilized nanoparticle formulation, ipilimumab, Stereotactic Radiosurgery (SRS), Stereotactic Radiation, ozurdex, letrozole, AG-013736 (axitinib), filgrastim, crizotinib, cediranib maleate, cediranib, bortezomib, abraxane, vorinostat, vincristine, TRC105, rituximab, regorafenib, pembrolizumab, methotrexate, imatinib, Herceptin, tecentriq, oxaliplatin (OXA), lomustine, ixabepilone, CPT-11, CGC-11047, vinorelbine tartrat, tartrate, prednisone, nivolumab, fulvestrant, enzastaurin, doxil, AZD2014, AZD2281, AZD2171, AZD4547, AZD5363, AZD8931, Vitamin B12, Vitamin C, Vitamin D, Valproic acid, mitomycin C, Cediranib Maleate, lenalidomide, lapatinib, HAI Abraxane, HAI Irinotecan, GDC-0941, GDC-0449, GDC-0980, bicalutamide, xeliri, vandetanib, thalidomide, rapamycin, olaparib, NovoTTF100A, Navelbine, metmab, Imatinib Mesylate (Gleevec), ifosfamide, hydroxychloroquine, and GM-CSF.

Still other suitable conditions for treatment may include, e.g., Hemophilia, Synovitis, Hypertension, keloid, inflammation, Radiation Necrosis, and Neoplastic Meningitis. These and the conditions described above may be delivered by any suitable route, except where subretinal or another type of administration to the eye is specified.

In certain embodiments, patients receive a single dose of rAAV8.aVEGF administered subretinally. For example, this is particularly well suited for treatment of neovascular age-related macular degeneration, macular edema following retinal vein occlusion (RVO), diabetic macular edema (DME), diabetic retinophathy (non-proliferative diabetic retinopathy (NPDR), proliferative diabetic retinopathy (PDR) in patients with DME, diabetic retinopathy in patients with diabetic macular edema.

The dose of rAAV8.aVEGF administered to a patient is at least 1×10⁹ GC/eye to 1×10¹³ GC/eye, or at least 1×10¹⁰ GC/eye to about 7.5×10¹² GC/eye (as measured by oqPCR or ddPCR). However, other doses may be selected. For example, therapeutically effective subretinal doses of the rAAV8.aVEGF for patients may range from about 6.6×10⁹ GC/eye to about 6.6×10¹¹ GC/eye, most preferably, 6.6×10¹⁰ GC/eye, in an injection volume ranging from about 0.1 mL to about 0.5 mL, preferably in 0.1 to 0.15 mL (100-150 μl). In still other embodiments, therapeutically effective concentrations may be about 1×10⁵ concentration can be 1×10⁵ GC/μL to 1×10⁹ GC/μL, and the volume of injection for any GC concentration in that range can be from 10 μL to 300 μL.

In certain embodiments, patients may receive an rAAV8.aVEGF by subretinal administration by a retinal surgeon under local anesthesia. The procedure may involve standard 3 port pars plana vitrectomy with a core vitrectomy followed by subretinal delivery into the subretinal space by a subretinal cannula (36 to 41 gauge). In certain embodiments, 100 to 150 microliters of rAAV8.aVEGF will be delivered.

In some embodiments, rAAV8.aVEGF is administered in combination with one or more therapies for the treatment of wetAMD or another selected disorder. In some embodiments, rAAV.aVEGF is administered in combination with laser coagulation, photodynamic therapy with verteporfin, and intravitreal with anti-VEGF agent, including but not limited to pegaptanib, ranibizumab, aflibercept, or bevacizumab.

In certain embodiments, patients for rAAV8.aVEGF therapy may include those which have previously responded to conventional anti-VEGF antibody (Fab) treatment.

The goal of the gene therapy treatment of the invention is to slow or arrest the progression of retinal degeneration, and to slow or prevent loss of vision with minimal intervention/invasive procedures. In certain embodiments, the efficacy of the gene therapy treatment may be indicated by the elimination of or reduction in the number of rescue treatments using standard of care, for example, intravitreal injections with anti-VEGF agents, including but not limited to pegaptanib, ranibizumab, aflibercept, or bevacizumab.

In certain embodiments, efficacy by measured by one or more of the following: Vision change, visual acuity, including best corrected visual acuity measured by (BCVA) score, Snellen chard or Early Treatment Diabetic Retinopathy (ETDRS) visual acuity score, percentage of subjects losing or gaining measured by ETDRS, distance best corrected visual acuity, reading best corrected visual acuity, change in NEI Visual Functioning Questionnaire-25 (VFQ-25) score, questionnaire of vision-related quality of life, contrast sensitivity measured by Pelli-Robson charts; low-contrast visual acuity on Electronic Visual Acuity Tester; peripheral visual field as measured by Goldmann visual field, mean angle opening distance and trabecular-iris spur area measured by Heidelberg Slit-Lamp Optical Coherence Tomography, Preferential-Hyperacuity-Perimeter (PHP) testing of Age Related Macular Degeneration (AMD) by characterizing central and paracentral metamorphopsia, retinal sensitivity (mfERG, Nidek MP-1 microperimetry), Visual Analog Scale (VAS), Macular Mapping Test, electrophysiological changes, including electroretinogram (ERG), pattern electroretinography (PERG) and full field (or flash) electroretinography (ffERG), multifocal electroretinography (mfERG), mfERG central ring amplitude density; mean retinal sensitivity (dB) in three concentric rings (4°, 8° & 12°), visual evoked potential (VEP): ECG parameters included PR interval, QRS interval, and corrected QT interval using Fridericia's formula (QTcF). Anatomical changes, including regression of NVE (retinal neovascularization), CNVM (Choroidal Neovascular Membranes), changes measured using optical coherence topography (OCT), including macular volume, macular thickness, central macular subfield thickness, retinal volume (inner retinal volume and outer retinal volume), retinal thickness, central retinal thickness, central subfield retinal thickness (CSRT), subfoveal retinal thickness (SRT), foveal thickness, maximum diameter of foveal avascular zone, integrity of retinal layers, external limiting membrane (ELM) integrity, ellipsoidal line/band integrity, lens status, lens opacity, neovascular membrane regression percentage measured by Optical coherence tomography angiography (OCTA), degree of integrity of the photoreceptors in the inner/outer segments layer in the 1 mm centered in the fovea. Optionally, during trial, AMD lesion size and leakage may be by fluorescein angiography, change in total lesion size and CNV (choroidal neovascularization) size by fluorescein angiography (FA) and Indocyanine green angiography (ICG), active CNV leakage which may include subretinal fluid or hemorrhage, area of leakage, area of macular leakage, change in percentage of lesion hemorrhage, change in drusen size, amount of fluid, intra-retinal cystoid changes (IRCs) volume, vessel density, presence of intra/sub-retinal fluid, sub-retinal fluid (SRF) height and diameter, intraretinal fluid volume, anterior chamber reaction, chorioretinal perfusion (ICG), development of geographic atrophy (GA) as detected by fundus photography (FP) and/or fundus autofluorescence (AF), presence and extension of capillary occlusion, peripheral retinal ischemia, macular sensitivity using microperimetry, neovascularization of the iris, neovascularization of the angle, diabetic retinopathy.

In certain embodiments, subretinal and/or intra-retinal injection of the AAV8.aVEGF results in plasma and serum levels free of the aVEGF.

In certain embodiments, efficacy may be monitored by measuring BCVA (Best-Corrected Visual Acuity), intraocular pressure, slit lamp biomicroscopy, indirect ophthalmoscopy, SD-OCT (SD-Optical Coherence Tomography). Signs of vision loss, infection, inflammation and other safety events, including retinal detachment may also be monitored.

SD-OCT is a useful non-invasive, in vivo cross-sectional retinal microscopy technique. Suitable equipment is commercially available. See, e.g., Spectralis OCT, Heidelberg Engineering, Carlsbad, CA. In brief, this technique may be performed by dilating pupils. En face retinal imaging can be performed with near infrared (NIR) reflectance (REF) and/or with NIR fundus autofluorescence (FAF) using the scanning laser ophthalmoscope of this imaging system. Spectral domain optical coherence tomography scanning can be performed with 9 mm long horizontal and vertical cross-sections through the fovea and overlapping 30×25 mm raster scans extending into the near midperiphery. The parameters may be modified as needed, or other suitable parameters determined comparable.

Retinal function can be evaluated by a full-field electroretinogram (ERG). An ERG is a mass electrical potential generated by the retina in response to light stimulus. Usually, it is recorded by an electrode in contact with the corneal surface. Electroretinograms can be conducted in accordance with the recommendations set by the International Society for Clinical Electrophysiology of Vision (ISCEV; McCulloch, Doc Ophthalmol. 2015 February; 130 (1): 1-12. 2015). In summary, an electroretinogram (ERG) is usually generated when all retinal cells actively respond to a flash stimulation (a dark-adapted animal, moderate to intense flash). The 2 components are the following: •a-wave: cornea-negative signal, first after the flash. Origin: photoreceptor photocurrent, the most direct signature of photoreceptor function. •b-wave: cornea-positive signal following the a-wave generated mostly by on-bipolar cells (second order neurons downstream from photoreceptors). In the examples described below, the following International Society for Clinical Electrophysiology of Vision (ISCEV) standard and additional protocols were used. However, these parameters may be adjusted as needed or required. Dark-adapted rod ERG: Stimulus intensity: 0.01 to 0.02 cd s m⁻². Response: b-wave only, no a-wave. Source: rod “on” bipolar cells (second order neurons driven by input from rods). Meaning: a measure of rod function. Dark-adapted standard flash ERG: Stimulus intensity: 3 cd s m⁻². Response: combined rod-cone a- and b-waves; 60% to 70% of the signal being generated by the rod-driven pathway. Source: photoreceptors, both rods and cones (a-wave); higher order neurons driven by both rods and cones. Meaning: a measure of mostly rod function; less sensitive to the state of dark adaptation and less variable than the “dim flash” response. Dark-adapted bright flash ERG: Stimulus intensity: 10 cd s m⁻². Response and meaning: same as for the “standard flash” response, but bright flash response is larger in magnitude and may be less variable. Light-adapted standard flash cone ERG: Stimulus intensity: 3 cd s m⁻², delivered in presence of 30 cd m⁻² background light after 5 minutes of light adaptation. Response: a- and b-waves generated by cone-driven pathways. Meaning: in presence of background light which completely desensitizes rods the ERG is produced exclusively by cones and cone-driven secondary retinal neurons and is a measure of the cone function. Light-adapted bright flash cone ERG (in addition to the ISCEV standard): Stimulus intensity: 10 cd s m⁻², delivered in presence of 30 cd m⁻² background light after 5 minutes of light adaptation. Response and meaning: cone-driven ERG as in case of the “Standard cone ERG”, but of greater magnitude and potentially less variable. ERG measures (a-wave amplitude, a-wave implicit time, b-wave amplitude, b-wave implicit time) were summarized using mean and standard deviation (SD) for treated eyes and control eye.

Another measure of efficacy may include a lack of thickening of the retina.

As illustrated in the examples below, administration of 1×10¹⁰ GC/eye of an AAV8.aVEFG vector causes no impairment to retinal function. This dose is not a limitation on the therapeutically effective amounts which can be administered.

### Measuring Clinical Objectives

Safety of the gene therapy vector after administration can be assessed by the number of adverse events, changes noted on physical examination, and/or clinical laboratory parameters assessed at multiple time points up to about 36 months post vector administration. Although physiological effect may be observed earlier, e.g., in about 1 day to one week, in one embodiment, steady state levels expression levels are reached by about 12 weeks.

Improvement/efficacy resulting from rAAV.aVEGF administration can be assessed as a defined mean change in baseline in visual acuity at about 12 weeks, 12 months, 24 months, 36 months, or at other desired time points. Other improvements/efficacy can be assessed as mean change from baseline in central retinal thickness as measured by spectral domain optical coherence tomography (SD-OCT) at 12, 24 and 36 months.

In some embodiments, treatment with rAAV.aVEGF results in a 5%, 10%, 15%, 20%, 30%, 40%, 50% or more increase in visual acuity from baseline. In some embodiments, treatment with rAAV.aVEGF results in a decrease, e.g., about 5%, about 10%, about 15%, about 20%, about 30%, about 40%, about 50% or more decrease in central retinal thickness. In other embodiments, the central retinal thickness is stable, i.e., no increase in central retinal thickness. In certain embodiments, a measure of efficacy includes stabilizing retinal thickness, and/or stabilizing/decreasing) exudate and/or drusen.

In one embodiment, expression may be observed as early as about 8 hours to about 24 hours post-dosing. One or more of the desired clinical effects described above may be observed within several days to several weeks post-dosing.

The invention is illustrated by the examples below which demonstrate that subretinal administration of an rAAV8.aVEGF vector results in gene transfer throughout the retina, and expression of anti-VEGF Fab throughout the retina and in the vitreous and anterior chamber fluids. This result is surprising in view of prior art gene therapy studies that demonstrated that gene transfer spreads laterally outside of the original injection bleb but remains confined to those expanded boundaries and did not achieve gene transfer and transgene expression outside this expanded area of injection (the “bleb” formed in the retina at the injection site); and offers an advantage over standard of care treatment for nAMD in that a single administration of the rAAV8.aVEGF vector should result in (i) continuous delivery of the effective amounts of the VEGF inhibitor throughout the retina which may in turn improve performance as compared to repeated IVT administrations of high dose boluses of the VEGF inhibitor that dissipate over time; and (ii) avoidance of repeated ocular injections which pose additional risks and inconvenience to patients. Each aspect may improve therapeutic outcome.

### EXAMPLES

The following abbreviations are used in the specification: AAV refers to Adeno-Associated Virus. ACF refers to Anterior Chamber Fluid. Ad5 refers to Adenovirus type 5. AE refers to Adverse Event. AMD refers to Age-Related Macular Degeneration. BCA refers to Bicinchoninic Acid. BCVA refers to Best-Corrected Visual Acuity. BH refers to Bulk Harvest. BI refers to Bulk Drug Substance Intermediate. BP refers to Base pairs. CB refers to Chicken Beta Actin Promoter. CB7 refers to a hybrid CMV Enhancer (C4) and Chicken β-Actin Promoter. CBC refers to Complete Blood Count. CI refers to chicken β-Actin Intron. CMC refers to Chemistry, Manufacturing and Control. CMO refers to Contract Manufacturing Organization. CMV refers to Cytomegalovirus. CNV refers to Choroidal Neovascularization. CS-10 refers to Corning 10-layer CellSTACKs® plates. ddPCR refers to Droplet Digital Polymerase Chain Reaction. DLS refers to Dynamic Light Scattering. DMEM refers to Dulbecco's Modified Eagle Medium. DNA refers to deoxyribonucleic Acid. DP refers to Drug Product. ELISA refers to Enzyme-Linked Immunosorbent Assay. ERG refers to electroretinogram. ELISPOT refers to Enzyme Linked Immunospot. Fab refers to Antigen-Binding Fragment. FBS refers to Fetal Bovine Serum. GC refers to Genome Copies. g refers to gram. GLP refers to Good Laboratory Practices. GMP refers to Good Manufacturing Practices. HEK293 refers to Human Embryonic Kidney Cells. HCP refers to Host Cell Protein. HS-36 refers to Corning 36-layer HYPERStacks®. ICH refers to International Conference on Harmonization. IND refers to Investigational New Drug. IP refers to In-Process. ITR refers to Inverted Terminal Repeat. IU refers to Infectious Unit. IV refers to Intravenous. IVT refers to Intravitreal. KDa refers to KiloDalton. Kg refers to Kilogram. LOQ refers to Limit of Quantification. Lucentis® is a brand name Ranibizumab. MCB refers to Master Cell Bank. MED refers to Minimally Effective Dose. μl refers to microliter. mL refers to milliliter. Mm refers to millimeter. mRNA refers to Messenger RNA. MS refers to Mass Spectrometry. Ng refers to Nanogram. NHP refers to Non-Human Primate. OCT refers to Optical Coherence Tomography. oqPCR refers to Optimized Quantitative Polymerase Chain Reaction. PCR refers to Polymerase Chain Reaction. PD refers to Pharmacodynamics. popPK refers to Population Pharmacokinetics. PEI refers to Polyethylenimine. PK refers to Pharmacokinetics. POC refers to Proof-Of-Concept. PRN refers to pro re nata (as needed). QA refers to Quality Assurance. qPCR refers to Quantitative Polymerase Chain Reaction. rAAV refers to Recombinant Adeno-Associated Virus. RBG refers to Rabbit Beta-Globin. RPE refers to Retinal Pigment Epithelium. S-36 refers to HYPERstack®36-layer. SEND refers to Standards for Exchange of Nonclinical Data. SOC refers to Standard of Care. SOP refers to Standard Operating Procedure. TCID50 refers to Tissue Culture Infectious Dose 50%. TFF refers to Tangential Flow Filtration. μL refers to Microliter. VA refers to Visual Acuity. VEGF refers to Vascular Endothelial Growth Factor. WAMD refers to Wet Age-Related Macular Degeneration. YAG refers to Yttrium-aluminum-garnet.

### Example 1: Treating Human Subjects

This Example relates to a gene therapy treatment for patients with neovascular (wet) age-related macular degeneration (nAMD). In this example, the gene therapy vector, rAAV8.aVEGF, a replication deficient adeno-associated viral vector 8 (AAV8) carrying a coding sequence for a soluble anti-VEGF Fab protein is administered to patients with nAMD. The goal of the gene therapy treatment is to slow or arrest the progression of retinal degeneration and to slow or prevent loss of vision with minimal intervention/invasive procedures.

### A. Gene Therapy Vector

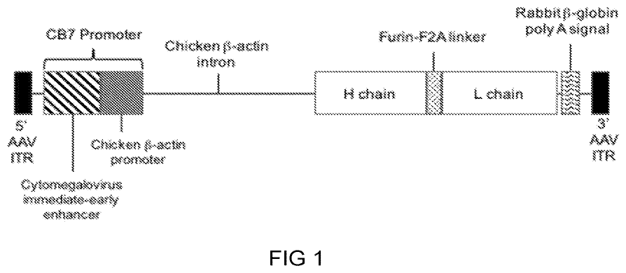

The generation of several rAAV8.aVEGF gene therapy vectors is described in Example 2 herein. Moreover, a schematic representation of the rAAV8.aVEGF vector genome is shown in FIG. 1. rAAV8.aVEGF is a non-replicating recombinant AAV8 viral vector containing a transgene that leads to the production of a human anti-vascular endothelial growth factor (anti-VEGF) antigen binding antibody fragment (Fab). The gene cassette is flanked by the AAV2 inverted terminal repeats (ITRs). Expression from the cassette is driven by a CB7 promoter, a hybrid of a cytomegalovirus immediate-early enhancer and the chicken β-actin promoter. Transcription from this promoter is enhanced by the presence of the chicken β-actin intron. The polyadenylation signal for the expression cassette is from the rabbit β-globin gene. The nucleic acid sequences coding for the heavy and light chains of anti-VEGF Fab are separated by a self-cleaving furin (F)/F2A linker. The incorporation of the furin-F2A linker ensures expression of about equal amounts of the heavy and the light chain polypeptides.

The final product is supplied as a frozen solution of the AAV vector active ingredient in a formulation buffer in Crystal Zenith® vials sealed with latex-free rubber stoppers and aluminum flip-off seals. Vials are stored at ≤−60° C.

### B. Dosing & Route of Administration

A volume of 250 μL of rAAV8.aVEGF is administered as a single dose via subretinal delivery in the eye of a subject in need of treatment. The subject receives a dose of 3×10⁹ GC/eye, 1×10¹⁰ GC/eye, or 6×10¹⁰ GC/eye.

rAAV8.aVEGF is administered by a single subretinal delivery by a retinal surgeon with the subject under local anesthesia. The procedure involves a standard 3-port pars plana vitrectomy with a core vitrectomy followed by subretinal delivery of rAAV8.aVEGF into the subretinal space by a subretinal cannula (38 gauge). The delivery is automated via the vitrectomy machine to deliver 250 μL to the subretinal space.

rAAV8.aVEGF can be administered in combination with one or more therapies for the treatment of wet AMD. For example, rAAV8.aVEGF is administered in combination with laser coagulation, photodynamic therapy with verteporfin, and intravitreal with anti-VEGF agent, including but not limited to pegaptanib, ranibizumab, aflibercept, or bevacizumab.

Starting at about 4 weeks post-rAAV8.aVEGF administration, a patient may receive intravitreal ranibizumab rescue therapy in the affected eye.

### C. Patient Subpopulations

Suitable patients may include those:
Having a diagnosis of nAMD;
Responsive to anti-VEGF therapy;
Requiring frequent injections of anti-VEGF therapy;
Males or females aged 50 years or above;
Having a BCVA≤20/100 and ≥20/400 (≤65 and ≥35 ETDRS letters) in the affected eye;
Having a BCVA between ≤20/63 and ≥20/400 (≤75 and ≥35 ETDRS letters);
Having a documented diagnosis of subfoveal CNV secondary to AMD in the affected eye;
Having CNV lesion characteristics as follows: lesion size less than 10 disc areas (typical disc area is 2.54 mm²), blood and/or scar <50% of the lesion size;
Having received at least 4 intravitreal injections of an anti-VEGF agent for treatment of nAMD in the affected eye in the 8 months (or less) prior to treatment, with anatomical response documented on SD-OCT; and/or
Having subretinal or intraretinal fluid present in the affected eye, evidenced on SD-OCT.
Prior to treatment, patients are screened and one or more of the following criteria may indicate this therapy is not suitable for the patient:
CNV or macular edema in the affected eye secondary to any causes other than AMD;
Blood occupying ≥50% of the AMD lesion or blood >1.0 mm² underlying the fovea in the affected eye;
Any condition preventing VA improvement in the affected eye, e.g., fibrosis, atrophy, or retinal epithelial tear in the center of the fovea;
Active or history of retinal detachment in the affected eye;
Advanced glaucoma in the affected eye;
Any condition in the affected eye that may increase the risk to the subject, require either medical or surgical intervention to prevent or treat vision loss, or interfere with study procedures or assessments;
History of intraocular surgery in the affected eye within 12 weeks prior to screening (Yttrium aluminum garnet capsulotomy may be permitted if performed >10 weeks prior to the screening visit.);
History of intravitreal therapy in the affected eye, such as intravitreal steroid injection or investigational product, other than anti-VEGF therapy, in the 6 months prior to screening;
Presence of an implant in the affected eye at screening (excluding intraocular lens).
History of malignancy requiring chemotherapy and/or radiation in the 5 years prior to screening (Localized basal cell carcinoma may be permitted.);
History of therapy known to have caused retinal toxicity, or concominant therapy with any drug that may affect visual acuity or with known retinal toxicity, e.g, chloroquine or hydroxychloroquine;
Ocular or periocular infection in the affected eye that may interfere with the surgical procedure;
Myocardial infarction, cerebrovascular accident, or transient ischemic attacks within the past 6 months of treatment;
Uncontrolled hypertension (systolic blood pressure [BP]>180 mmHg, diastolic BP>100 mmHg) despite maximal medical treatment;
Any concomitant treatment that may interfere with ocular surgical procedure or healing process;
Known hypersensitivity to ranibizumab or any of its components or past hypersensitivity to agents like rAAV8.aVEGF;
Any serious or unstable medical or psychological condition that, in the opinion of the Investigator, would compromise the subject's safety or successful participation in the study.
Aspartate aminotransferase (AST)/alanine aminotransferase (ALT)>2.5× upper limit of normal (ULN)
Total bilirubin>1.5×ULN unless the subject has a previously known history of Gilbert's syndrome and a fractionated bilirubin that shows conjugated bilirubin<35% of total bilirubin
Prothrombin time (PT)>1.5×ULN
Hemoglobin<10 g/dL for male subjects and <9 g/dL for female subjects
Platelets<100×10³/μL
Estimated glomerular filtration rate (GFR)<30 mL/min/1.73 m²

Starting at about 4 weeks post-rAAV8.aVEGF administration, a patient may receive intravitreal ranibizumab rescue therapy in the affected eye for disease activity if 1 or more of the following rescue criteria apply:
Vision loss of ≥5 letters (per Best Corrected Visual Acuity [BCVA]) associated with accumulation of retinal fluid on Spectral Domain Optical Coherence Tomography (SD-OCT)
Choroidal neovascularization (CNV)-related increased, new, or persistent subretinal or intraretinal fluid on SD-OCT
New ocular hemorrhage

Further rescue injections may be deferred per the Investigator's discretion if one of the following sets of findings occur:
Visual acuity is 20/20 or better and central retinal thickness is “normal” as assessed by SD-OCT, or
Visual acuity and SD-OCT are stable after 2 consecutive injections.

If injections are deferred, they are resumed if visual acuity or SD-OCT get worse per the criteria above.

### D. Measuring Clinical Objectives

Primary clinical objectives include slowing or arresting the progression of retinal degeneration and slowing or preventing loss of vision. Clinical objectives are indicated by the elimination of or reduction in the number of rescue treatments using standard of care, for example, intravitreal injections with anti-VEGF agents, including but not limited to pegaptanib, ranibizumab, aflibercept, or bevacizumab. Clinical objectives are also indicated by a decrease or prevention of vision loss and/or a decrease or prevention of retinal detachment.

Clinical objectives are determined by measuring BCVA (Best-Corrected Visual Acuity), intraocular pressure, slit lamp biomicroscopy, indirect ophthalmoscopy, and/or SD-OCT (SD-Optical Coherence Tomography). In particular, clinical objectives are determined by measuring mean change from baseline in BCVA over time, measuring the gain or loss of ≥15 letters compared to baseline as per BCVA, measuring mean change from baseline in CRT as measured by SD-OCT over time, measuring mean number of ranibizumab rescue injections over time, measuring time to 1st rescue ranibizumab injection, measuring mean change from baseline in CNV and lesion size and leakage area based on FA over time, measuring mean change from baseline in aqueous aVEGF protein over time, performing vector shedding analysis in serum and urine, and/or measuring immunogenicity to rAAV.aVEGF, i.e., measuring Nabs to AAV, measuring binding antibodies to AAV, measuring antibodies to aVEGF, and/or performing ELISpot.

Clinical objectives are also determined by measuring the mean change from baseline over time in area of geographic atrophy per fundus autofluorescence (FAF), measuring the incidence of new area of geographic atrophy by FAF (in subjects with no geographic atrophy at baseline, measuring the proportion of subjects gaining or losing≥5 and ≥10 letters, respectively, compared with baseline as per BCVA, measuring the proportion of subjects who have a reduction of 50% in rescue injections compared with previous year, measuring the proportion of subjects with no fluid on SD-OCT.

Improvement/efficacy resulting from rAAV.aVEGF administration can be assessed as a defined mean change in baseline in visual acuity at about 4 weeks, 12 weeks, 6 months, 12 months, 24 months, 36 months, or at other desired timepoints. Treatment with rAAV.aVEGF can result in a 5%, 10%, 15%, 20%, 30%, 40%, 50% or more increase in visual acuity from baseline. Improvements/efficacy can be assessed as mean change from baseline in central retinal thickness (CRT) as measured by spectral domain optical coherence tomography (SD-OCT) at 4 weeks, 12 weeks, 6 months, 12 months, 24 months and 36 months. Treatment with rAAV.aVEGF can result in a 5%, 10%, 15%, 20%, 30%, 40%, 50% or more increase central retinal thickness from baseline.

### Example 2: Generation of AAV8.CMV.aVEGF

Each of the aVEGF vectors described herein include an expression cassette including a promoter which drives expression of the anti-VEGF Fab heavy chain and light chain, each of which has an IL2 leader sequence. The Fab coding sequence in the vector genomes carried by the rAAV in the tested composition (suspension) were designed to be identical. The expression cassette is flanked by a 5′ AAV2 ITR and a 3′ AAV2 ITR. Each of the tested vector genomes contains a coding sequence variant for the same anti-VEGF Fab (previously designated aVEGF-Arg or aVEGF-R). In certain embodiments, the expressed aVEGF Fab is a homogenous population. In certain embodiments, the expressed aVEGF Fab has heterogeneity at the heavy chain carboxy terminus. The open reading frames for the IL2-aVEGF heavy chain and IL2-aVEGF light chain were separated by an encoded furin cleavage site/F2A linker to promote equal molar expression of both, heavy and light chains. This results in expression of an aVEGF heavy chain which optionally further contains 0, 1, 2, 3 or 4 amino acids at its carboxy terminus. an arginine, arginine-lysine, arginine-lysine-arginine, or arginine-lysine-arginine-arginine at its carboxy terminus.

Various coding sequences are designated aVEGFv1, v2, etc. These vector genomes are provided in the Sequence Listing, which is incorporated by reference.

The following elements to be included in the transgene cassette in AAV2/8 vector for expression of anti-VEGF Fab in mice were evaluated.
7 different promoters (98 male C57BL/6 mice; Jackson Laboratories) were assessed using a convenient antibody (F16) expressed from AAV2/8. Expression of FI6 mAb was measured by ELISA against hemagglutinin (HA) protein;
2 different leader peptides (28 male C57BL/6 mice; Jackson Laboratories). Expression of anti-VEGF Fab was measured by ELISA against VEGF;
3 different light-heavy chain separators (42 male C57BL/6 mice; Jackson. Laboratories) were evaluated using the following vectors.

| | | No. of animals | | |
|---|---|---|---|---|
| | | Dose (GC/eye) | | |
| Group | Treatment | 1.00 × 10⁹ | 5.00 × 10⁹ | ROA |
| 1 | AAV2/8.CMV.PI.aVEGFv7.EMCVIRES.Fab. | 7 | 7 | Subretinal |
| | SV40; the sequence of the expression cassette | | | |
| | is provided in SEQ ID NO: 45 | | | |
| 2 | AAV2/8.CMV.PI.aVEGFv7.FMDV1IRES.Fab. | 7 | 7 | Subretinal |
| | SV40; the sequence of the expression | | | |
| | cassette is provided in SEQ ID NO: 46 | | | |
| 3 | AAV2/8.CMV.PI.aVEGFv7.cMycIRES.Fab.SV40; | 7 | 7 | Subretinal |
| | the sequence of the expression cassette is | | | |
| | provided in SEQ ID NO: 47 | | | |

Abbreviation:
GC = genome copies;
No. = number;
ROA = route of administration.

13 different coding sequences (182 male C57BL/6 mice; Jackson Laboratories). Expression of anti-VEGF Fab was measured by ELISA against VEGF.

Vectors were delivered into subretinal space of the mouse eye. Expression of reporter genes was determined by enzyme-linked immunosorbent assay (ELISA).

Seven different promoters were evaluated in another study: 3 viral (cytomegalovirus [CMV], thymidine kinase [TK], simian virus [SV40]), 3 non-viral (phosphoglycerate kinase [PGK], human elongation factor-1α [EF1α], ubiquitin C [UbC]), and 1 hybrid (chicken β-actin [CB7]) promoters.

Two different leader peptides were also evaluated using rAAV8 vectors having identical vector elements and the same coding sequence, i.e., v3, with the exception of the leader sequence (interleukin-2 vs the serpin leader). AAV2/8=adeno associated virus (AAV) capsid type 8 with AAV2 inverted terminal repeats flanking the transgene; amd201Lead=anti-VEGF Fab with IL2 leader sequence and Furin F2A as light-heavy chain separator; amd201altLead=anti-VEGF Fab with SF1 leader sequence and Furin F2A as light-heavy chain separator; CB7=chicken β actin promoter; CI=chimeric intron; rBG=rabbit β globin polyadenylation sequence.

Three different internal ribosome entry site (IRES) sequences separating heavy and light chains of anti-VEGF Fab were evaluated in another study. These IRES sequences were derived from encephalomyocarditis virus (EMCV), cMyc, and foot-and-mouth disease virus 1 (FMDV1). In this study, the vectors were identical except for the light and heavy chain separators (EMC, FMDV1 and cMyc). AAV2/8=adeno associated virus (AAV) capsid type 8 with AAV2 inverted terminal repeats flanking the transgene; amd201=codon variant of anti-VEGF Fab with IL2 leader sequence; CMV=cytomegalovirus promoter; EMCV=encephalomyocarditis virus; Fab=fragment antigen-binding region; FMDV1=foot and mouth disease virus 1; IRES=internal ribosome entry site; PI=Promega intron; SV40=simian virus polyadenylation sequence

In another study, thirteen different coding sequences for the anti-VEGF Fab were evaluated. The overall coding sequence variance was between approximately 20% and 30%. The vectors are described in the following Table.

Vectors with Different Coding Sequences Used. The SEQ ID NO for the expression cassettes is provided in the following table:

| | SEQ ID NO: |
|---|---|
| Vector | (vector genome) |
| AAV2/8.CB7.CI.aVEGFv4.rBG | 35 |
| AAV2/8.CB7.CI.aVEGFv5.rBG | 36 |
| AAV2/8.CB7.CI.aVEGFv1.rBG | 34 |
| AAV2/8.CB7.CI.aVEGFv2.rBG: | 3 |
| AAV2/8.CB7.CI.aVEGFv6.rBG | 37 |
| AAV2/8.CB7.CI.aVEGFv7.rBG | 38 |
| AAV2/8.CB7.CI.aVEGFv8.rBG | 39 |
| AAV2/8.CB7.CI.aVEGFv9.rBG | 40 |
| AAV2/8.CB7.CI.aVEGFv10.rBG | 41 |
| AAV2/8.CB7.CI.aVEGFv11.rBG | 42 |
| AAV2/8.CB7.CI.aVEGFv12.rBG | 43 |
| AAV2/8.CB7.CI.aVEGFv3.rBG | 14 |
| AAV2/8.CB7.CI.aVEGFv13.rBG | 44 |

Vectors in all studies were diluted in Dulbecco's phosphate-buffered saline (DPBS).

Animals were assigned into treatment groups and administered 1.00×10⁹ or 5.00×10⁹ genome copies (GC)/eye of AAV2/8 vectors into the right eye. The left eye was used as an untreated control. Vectors were administered subretinally in a total volume of 1 μL.

### A. Subretinal Injections

Subretinal injections were conducted using aseptic technique and sterile dissecting instruments. Animals were anesthetized with ketamine/xylazine or 3% to 5% isoflurane and administered meloxicam. Animals were then placed under a dissection microscope with the eye to be injected under view (using a 15× magnification). The temporal conjunctiva was grasped with jeweler's forceps and carefully cut down to the sclera using the tip of Vannas iridotomy scissors. Conjunctival peritomy was conducted by introducing the lower lip of the scissors through the incision and extending circumferentially both superiorly and inferiorly of the conjunctiva. Any conjunctival debris was carefully removed from the surface of the sclera. The conjunctiva adjacent to the cornea was grasped with the forceps, providing traction to rotate the globe and allow optimal surgical exposure. Using a 30½-gauge needle, a small incision, large enough to allow the blunt-tip needle to pass through, was made.

The tip of a 33-gauge blunt-tip needle mounted on a Hamilton auto-injector syringe was introduced into the incision tangentially to the surface of the globe. The needle was passed along the inner surface of the sclera with the tip entering approximately 1 mm. The 33-gauge needle passed through the sclera and choroid and then terminated in the subretinal space. Up to 1 μL of vector was delivered. Once the procedure was completed, antibiotic ophthalmic ointment was applied to the eye.

### B. Assay Methods

The collected eyes were homogenized by placing entire eyeball into a conical tube with stainless steel beads and 200 μL of cocktail containing protein lysis and extraction buffer (RIPA) and cOmplete™, Mini Protease Inhibitor Cocktail tablets (1 tablet/10 mL of RIPA buffer). The eyes were homogenized for at least 2 minutes in a TissueLyser (Qiagen, USA) or until fully homogenized. Homogenate was centrifuged for 20 minutes at 12000 RPM at 4° C. in a cold room. The supernatants were transferred into fresh tubes and used in analytical assays.

### Determination of Protein Concentrations in Eye Homogenates

Protein concentration in eye homogenate was determined using Pierce™ BCA Protein Assay Kit (Thermo Fisher Scientific) per the manufacturer's instructions. Equal amounts of protein in all samples were used in ELISA.

### Enzyme-Linked Immunosorbent Assay

Ninety-six-well, round-bottom plates were coated with 2 μg/mL of HA A-Beijing or 1 μg/mL VEGF overnight at 4° C. After coating, the plates were washed 5 times with 200 μL of phosphate-buffered saline (PBS) with 0.05% Tween-20 (PBS-T) using a 405 TS Washer (BioTek Instruments, Winooski, VT). Plates were then blocked with 200 μL/well of 1% bovine serum albumin (BSA) at room temperature (RT) for 1 hour. After washing (as described), 100 μL/well of sample was loaded into duplicate wells and incubated at 37° C. for 1 hour. Following incubation, the plates were washed (as described) and then blocked with 1% BSA at RT for 1 hour. After washing (as described), 100 μL/well of the primary antibody was added and incubated at RT for 1 hour. Wells were then washed (as described) and incubated with 100 μL/well of the secondary antibody at RT for 1 hour. Following a final wash (as described), 150 μL/well of 3,3′,5,5′-tetramethylbenzidine, a detection substrate, was added and incubated at RT for 30 minutes protected from light. The reaction was stopped with 50 μL/well of 2N H₂SO₄. The plates were then read at the excitation/emission of 450 nm/540 nm using spectrophotometer SpectraMax® M3 (Molecular Devices, Sunnyvale, CA).

The following primary antibodies were used: 1.0 mg/mL Goat Anti-Human IgG H&L (Biotin) preadsorbed at a 1:10000 dilution in PBS (Abcam 0.5 mg/mL); 0.5 mg/mL Goat Anti-Human IgG H&L (Biotin) preadsorbed at a 1:5000 dilution in PBS (Abcam, 1 mg/mL). The following secondary antibody was used: 1 mg/mL Streptavidin (HRP) at a 1:30000 dilution in PBS.

### Statistical Analyses

Average and standard deviation values for concentration of reporter genes for ELISA were calculated using Microsoft Office Excel 2010.

### C. Results

AAV2/8 vectors with 7 different promoters were evaluated for expression of FI6 mAb. Expression of FI6 mAb was not observed in any animal when promoter EF 1-α was used. Expression of FI6 mAb was low when promoters SV40.PI, PGK.PI and TK.PI were used. Promoters CMV.PI, CB7.CI, and UbC.PI demonstrated the highest expression of FI6 mAb. No expression was observed in the untreated left eye in any animal (data on file). AAV2/8 vectors with 2 different leader peptides were evaluated for expression of anti-VEGF Fab. Compared to aVEGFv7 with an IL2 leader, expression of anti-VEGF Fab at low dose was greater when leader peptide aVEGFv7 with SF2 leader was used. At high dose, the expression of anti-VEGF Fab was similar for both leader peptides. No expression was observed in the untreated left eye in any animal (data on file). AAV2/8 vectors with 3 different light-heavy chain separators were evaluated for expression of anti-VEGF Fab. Expression of anti-VEGF Fab was not observed in any animal when cMyc light-heavy chain separator was used. With EMCV and FMDV1 light-heavy chain separators, anti-VEGF Fab was expressed at low levels. No expression was observed in the untreated left eye in any animal (data on file).

AAV2/8 vectors with 13 different coding sequences were evaluated for expression of anti-VEGF Fab transgene product. When coding sequences aVEGFv4, aVEGFv5, aVEGFv6, aVEGFv7, aVEGFv8, and aVEGFv9 were used, expression of anti-VEGF Fab was low. Expression was higher with coding sequences aVEGFv13, aVEGFv10, aVEGFv11, and aVEGFv12. When coding sequences aVEGFv1, aVEGFv2, and aVEGFv3 were used, expression of anti-VEGF Fab was the highest. No expression was observed in the untreated left eye in any animal (data on file).

Each of the vectors encodes the same anti-VEGF transgene product. Based in part on these results, a single replication-defective, recombinant AAV8.aVEGF was selected for further development. This tert vector has an AAV8 capsid and a vector genome in which AAV2 ITRs flank a CB7 promoter, an intron, an anti-VEGF coding sequence selected from the coding sequence as described earlier and a rBG poly A sequence. This is termed the test vector (alternatively AAV2/8.aVEGF test vector or AAV8.aVEGF test vector) in the following examples, except where specifically specified otherwise.

### Example 3: Pharmacokinetic (PK) Study in Non-Human Primates

Macaques were used in this study because they are the closest species to humans for studying retinal diseases. Cynomolgus monkeys and humans have similar eye anatomy, including fovea. The dimensions of the eyes are comparable, which allows determination of the human dose based on relative retinal areas.

This study was conducted to select AAV2/8 vector for clinical development and to evaluate toxicity and immunogenicity of AAV2/8 vector and anti-VEGF Fab in cynomolgus monkeys. The study is ongoing. The results presented are based on the data collected at Month 10. Evaluation of toxicity of AAV2/8 vector and anti-VEGF Fab is described. Animals were administered AAV2/8 vectors subretinally. Toxicity was evaluated based on clinical observations, body weights, indirect ophthalmoscopy, spectral domain optical coherence tomography, hematology, coagulation, clinical chemistry, and gross pathologic findings. The only adverse finding related to AAV2/8 vector or anti-VEGF Fab was some thinning in outer nuclear layer localized to the injection site observed by spectral domain optical coherence tomography in several eyes of animals administered 1.00×10¹¹ GC/eye of AAV2/8 vectors.

Animals were assigned into 4 treatment groups. Animals were administered a single dose of 1.00×10¹¹ genome copies (GC)/eye of AAV2/8 vectors into each eye in a total volume of 100 μL. Vectors were administered subretinally (confirmed visually by appearance of a dome shaped retinal detachment/retinal bleb under microscope) into both eyes. The following table lists the tested vectors.

| | | Dose | No. of | ROA | |
|---|---|---|---|---|---|
| Group | Treatment | (GC/eye) | animals | OD | OS |

| | | | | |
|---|---|---|---|---|
| 2 | AAV2/8.UbC.PI.aVEGFv2.SV40 | 1.00 × 10¹¹ | 2 M, 2 F | Subretinal |
| 3 | AAV2/8.UbC.PI.aVEGFv3.SV40 | 1.00 × 10¹¹ | 2 M, 2 F | Subretinal |
| 5 | AAV2/8.CB7.CI.aVEGFv2aVEGFv2. | 1.00 × 10¹¹ | 2 M, 2 F | Subretinal |
| | rBG | | | |
| 6 | AAV2/8.CB7.CI.aVEGFv3.rBG | 1.00 × 10¹¹ | 2 M, 2 F | Subretinal |

Abbreviation:
F = female;
GC = genome copies;
M = male;
No. = number;
OD = right eye;
OS = left eye;
ROA = route of administration.

### Subretinal Injections

For subretinal injections, a needle was inserted through a trocar, introduced by sclerotomy, at the 2 or 10 o'clock position. The needle was advanced through the vitreous to penetrate the retina in the posterior pole. Under the microscopic control, 100 μL of test article was injected into the subretinal space. This was confirmed by appearance of a dome shaped retinal detachment/retinal bleb. If the first injection attempt did not result in retinal detachment, the cannula was moved to another site in the retina. The injection site may have resulted in a temporary scotoma. The injected solution was reabsorbed within a few hours by the retina. The retinal detachment was made in the peripheral retina and did not result in permanent blindness. The site of sclerotomy was sutured with absorbable suture and the eye dressed with PredG ointment or equivalent. Subconjunctival kenalog or equivalent was administered. Animals were observed daily and administered parenteral analgesics as needed. If vitreal inflammation appeared, animals were treated with topical atropine and PredG ointment or equivalent daily until symptoms resolved.

### Collection of Anterior Chamber Fluid

Animals were anesthetized and their head stabilized. Betadine 5% antiseptic solution and Proparacaine or equivalent were applied to each eye. An eye speculum was placed to allow access to the anterior chamber. The procedure was performed with a tuberculin syringe attached to a 27- to 30-gauge hypodermic needle. The eye was held steady with forceps or a cotton tip applicator on the nasal conjunctiva. The needle was inserted bevel up through the paralimbal peripheral clear cornea, anterior to the iris plane. Once the eye was entered, a sampler slowly withdrew the plunger of the syringe to aspirate the aqueous fluid. A maximum of 100 μL of anterior chamber fluid was collected. Once anterior chamber fluid was drained, the needle was withdrawn from the eye. The anterior chamber fluid was placed on wet ice until use or storage. After the procedure, topical flurbiprofen, PredG ointment, and antibiotic drops were applied to each eye. Anterior chamber fluid was collected on the following Study days (occasionally adjusted due to weekends, holidays or scheduling issues)
0, 15, 29, 43, 57, 71, 85, 120, 149, 183, 212, 247, 274, and 302.

### Spectral Domain Optical Coherence Tomography

Retinal structure (at a micron-level resolution) was evaluated by in vivo, non-invasive, cross-sectional retinal microscopy with SD-OCT (Spectralis OCT, Heidelberg Engineering, Carlsbad, CA). Pupils were dilated with phenylephrine 2.5% and tropicamide 1%. En-face retinal imaging was performed with near infrared (NIR) reflectance (REF) and in a subset of animals with NIR fundus autofluorescence (FAF) using the scanning laser ophthalmoscope of this imaging system. Spectral domain optical coherence tomography scanning was performed with 9 mm long horizontal and vertical cross-sections through the fovea and overlapping 30×25 mm raster scans extending into the near midperiphery. See, Aleman, Invest Ophthalmol Vis Sci. 2007 October; 48(10):4759-65.

### Enzyme-Linked Immunosorbent Assay (ELISA)

The ELISA was performed essentially as described for the mouse studies above. Ninety-six-well, round-bottom plates were coated with 1 μg/mL of VEGF for expression of anti-VEGF Fab. Plates were coated overnight at 4° C. After coating, the plates were washed 5 times with 200 μL of phosphate-buffered saline (PBS) with 0.05% Tween-20 (PBS-T) using a 405 TS Washer (BioTek Instruments, Winooski, VT). Plates were then blocked with 200 μL/well of 1% bovine serum albumin (BSA) at room temperature (RT) for 1 hour. After washing (as described), 100 μL/well of sample was loaded into duplicate wells and incubated at 37° C. for 1 hour. Following incubation, the plates were washed (as described) and then blocked with 1% BSA at RT for 1 hour. After washing (as described), 100 μL/well of the primary antibody was added and incubated at RT for 1 hour. Wells were then washed (as described) and incubated with 100 μL/well of the secondary antibody at RT for 1 hour.

Following a final wash (as described), 150 μL/well of 3,3′,5,5′-tetramethylbenzidine, a detection substrate, was added and incubated at RT for 30 minutes protected from light. The reaction was stopped with 50 μL/well of 2N H₂SO₄. The plates were then read at the excitation/emission of 450 nm/540 nm using spectrophotometer SpectraMax® M3 (Molecular Devices, Sunnyvale, CA). The following primary antibodies were used: 1.0 mg/mL Goat Anti-Human IgG H&L (Biotin) preadsorbed at a 1:10000 dilution in PBS; 0.5 mg/mL Goat Anti-Human IgG H&L (Biotin) preadsorbed at a 1:5000 dilution in PBS. The following secondary antibody was used: 1 mg/mL Streptavidin (HRP) at a 1:30000 dilution in PBS.

Average and standard deviation values for concentrations of the anti-VEGF Fab in anterior chamber fluid and blood were calculated using Microsoft Office Excel 2010.

### A Pharmacology Results

Four AAV vectors with different promoters and coding sequences were evaluated as described earlier in this example. Vectors were administered subretinally. Expression of anti-VEGF Fab was determined by enzyme-linked immunosorbent assay.

### Expression of Anti-VEGF Fab in Anterior Chamber Fluid

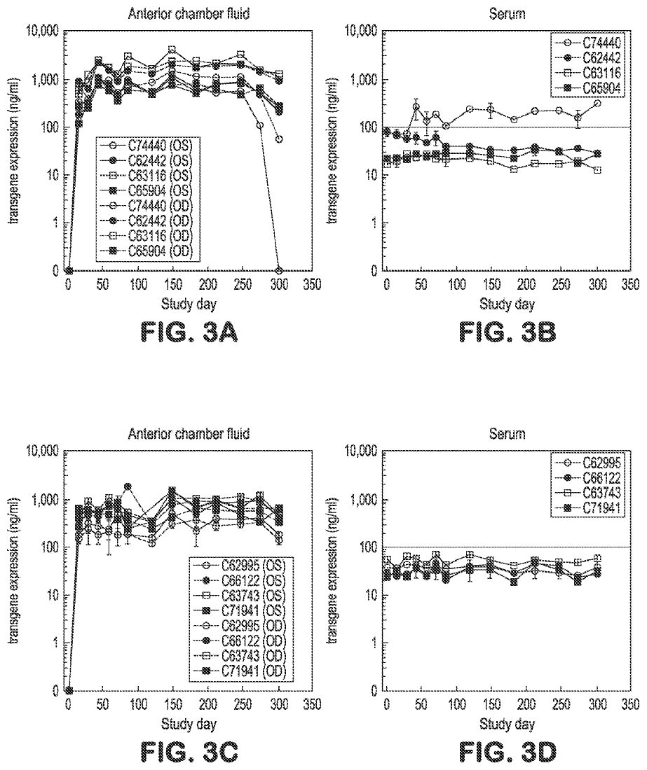

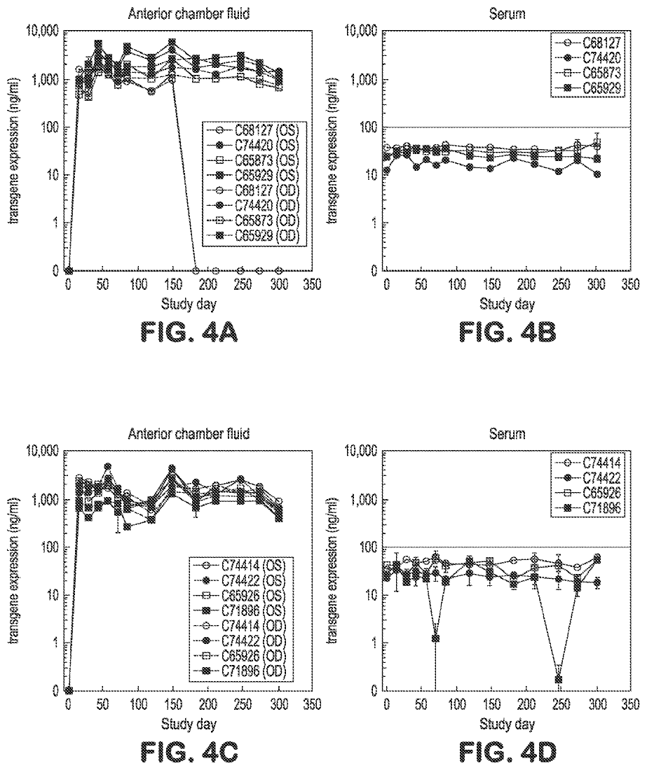

In anterior chamber fluid of animals in all groups, similar expression kinetics was observed (FIG. 3A-3D; FIG. 4A-4D). Onset of expression of the anti-VEGF Fab was rapid, generally within 7 days. Steady-state expression levels were achieved within 1 month. All except 2 animals continued to express the anti-VEGF Fab at steady-state levels until the last evaluated timepoint.

One animal in Group 2 (FIG. 3B) and 1 animal in Group 5 (FIG. 4A) lost expression of the anti-VEGF Fab. Loss of expression coincided with appearance of antibodies against the anti-VEGF Fab. No difference in expression of the anti-VEGF Fab between males and females and between the right and the left eye was observed.

Generally, vectors controlled by the CB7.CI promoter (FIG. 4A-4D) expressed the anti-VEGF Fab at higher levels than vectors controlled by the UbC.PI promoter (FIG. 3). Vector AAV2/8.CB7.CI.aVEGFv3aVEGFv3.rBG was selected as a primary vector for clinical development. This selection was based on expression level of transgene, better translatability of relative expression levels from mice to cynomolgus monkeys for aVEGFv3 coding sequence, and greater level of experience with CB7.CI promoter.

### Expression of Anti-VEGF Fab in Blood

In some patients administered a single IVT injection of Lucentis, ranibizumab was observed in serum (Xu, 2013). To determine if subretinal administration of AAV2/8 vector results in systemic exposure to the anti-VEGF Fab, its concentrations were measured in serum.

Expression of the anti-VEGF Fab was around the baseline levels in blood of all animals (FIGS. 3A-3D, FIGS. 4A-3D).

### B. Toxicology

Evaluation of toxicity of AAV2/8 vector and anti-VEGF Fab is described in this subpart B. Animals were administered AAV2/8 vectors subretinally. Toxicity was evaluated based on clinical observations, body weights, indirect ophthalmoscopy, spectral domain optical coherence tomography (SD-OCT), hematology, coagulation, clinical chemistry, and gross pathologic findings.

For each variable in each treatment group, the measurements at each time point were compared to the corresponding baseline values using Wilcoxon rank-sum test. The Wilcoxon rank-sum test is a nonparametric alternative to the two-sample t-test, which is based solely on the order in which the observations from the 2 samples fall. It is a preferable test for dataset with small sample size. Statistical significance was declared at the 0.05 level without the adjustment for multiple testing. The analysis was done using R program (version 3.3.1; cran.r-project.org/) with function “wilcox.test”.

The study is ongoing. The results presented are based on the data collected by Month 10. There were no mortalities in this study. No adverse clinical observations related to AAV2/8 vector or anti-VEGF Fab were noted in any animal. No clinically meaningful changes in body weight during the study were observed for any animal. No adverse observations related to AAV2/8 vector or anti-VEGF Fab were noted during indirect ophthalmoscopy in any animal.

### Spectral Domain Optical Coherence Tomography

All 4 animals (8 eyes) in Group 6 were imaged by SD OCT. Injected regions in the eyes that received the intermediate dose level of AAV8.aVEGF test vector (1.00×10¹¹ GC/eye) showed intermediate outcomes as compared to 1.00×10¹⁰ and 1.00×10¹² dose levels described in Example 7 (esp., subpart B). In two animals (Animal C71896 and Animal C65936), some thinning of ONL was observed (data on file). In addition, in 2 animals (Animal C74422 and Animal C74414), minimal changes were observed (data on file). No clinically significant changes in hematology, coagulation, or clinical chemistry parameters were observed in any animal.

No findings related to AAV2/8 vector or anti-VEGF Fab were observed in 2 animals sacrificed at Month 10. In Animal C65936, a liver mass was observed, which microscopically was focal chronic grade 3 inflammation. In Animal C74414, bilateral grade 3 lymphoid hyperplasia was observed. These finding were not related to AAV2/8 vector or anti-VEGF Fab. At a dose level of 1.00×10¹¹ GC/eye of AAV2/8 vector, the only findings related to AAV2/8 vector or anti-VEGF Fab were minimal vacuolation of the lens of the right eye of Animal C65926. Minimal perivascular mononuclear cell infiltrates around the vasculature of the right optic nerve in Animal C74414 were observed. In the same animal, a minimal mononuclear cell infiltrate in the subconjunctiva of the left eye and a minimal perivascular exocular mononuclear cell infiltrate in right eye were also noted.

The only adverse finding related to AAV2/8 vector or anti-VEGF Fab was some thinning in ONL localized to the injection site observed by SD OCT in several eyes of animals administered 1.00×10¹¹ GC/eye of AAV2/8 vectors.

### C. Immunology

In this section, evaluation of immunogenicity of AAV2/8 vector and anti-VEGF Fab is described. Vectors were administered subretinally as described earlier in this example. Immunogenicity was assessed by the presence of IgM and IgG antibodies against anti-VEGF Fab, neutralizing antibodies against AAV8 capsid, and cellular immune response against AAV2/8 vector and anti VEGF Fab.

To summarize, one animal each in Groups 2 and 5 had antibodies against anti-VEGF Fab, high levels of NAbs to AAV8 capsid, and T-cell responses. Both animals lost expression of anti-VEGF Fab. Animals with pre-existing NAbs to AAV8 capsid generally had increased response after administration of AAV2/8 vector when compared to animals without pre-existing NAbs. In some animals sacrificed at approximately Study day 300, NAbs to AAV8 capsid were observed in vitreous fluid. No antibodies against anti-VEGF Fab and no T-cell responses were observed in animals in Group 6. Only mild fluctuations in the levels of NAbs were observed in animals in Group 6 after administration of AAV2/8 vector.

Anti-VEGF Fab was expressed in all animals administered AAV2/8 vectors (Part A of this Example). Two animals (Animal C74440 and Animal C68127) lost expression of the anti-VEGF Fab. Loss of expression coincided with appearance of antibodies against the anti-VEGF Fab.

Overall, levels of IgM and IgG against anti-VEGF Fab were below baseline levels in anterior chamber fluid and serum. Increases above the baseline levels at some timepoints were observed in some animals. In 1 animal each in Groups 2 (Animal C74440) and 5 (Animal C68127), levels of IgG against anti-VEGF Fab in anterior chamber fluid increased above baseline levels approximately at approximately 6 months. The levels generally increased thereafter. In both animals, IgG against anti-VEGF Fab increased above the baseline levels in serum. These increases in IgG coincided with loss of expression of anti-VEGF Fab. Importantly, IgM and IgG against anti-VEGF Fab in animals in Group 6 were not detected or were below the baseline levels for the duration of the study.

In brief, the animal immune system permitted continued localized expression of anti-VEGF transgene product, despite the fact that the transgene product is a human antibody.

### Presence of Neutralizing Antibodies Against AAV8 Capsid

Baseline levels of NAbs against AAV8 capsid were determined in serum from blood samples collected on Study day 0. The limit of detection was a 1:5 dilution; titers of <5 were considered undetectable. In 2 of 16 animals (Animal C63116 and Animal C66122), pre-existing NAbs in serum were not observed. The levels of NAbs in these 2 animals following administration of AAV2/8 vectors remained below the limit detection or were low. In 14 of 16 animals, pre-existing NAbs were observed. In 11 of these animals, levels of NAbs fluctuated throughout the study. In 1 animal each in Groups 2 (Animal C74440) and 5 (Animal C68127), levels of NAbs following administration of AAV2/8 vector increased up to 256 and 128 two-fold dilutions respectively at two months. These increases in NAbs coincided with loss of expression of anti-VEGF Fab. In 6 sacrificed animals from Groups 2, 3 and 5, the presence of NAbs was evaluated in vitreous fluid. In 2 animals (Animal C63116 and Animal C66122), with undetectable NAb in serum time of sacrifice, NAbs were not present in vitreous fluid at sacrifice. In the remaining animals, levels of NAbs at sacrifice did not correlate with levels in serum at time of sacrifice. In all animals in Group 6, pre-existing NAbs were observed. The levels of NAbs in these animals fluctuated mildly throughout the study.

### T-Cell Responses to AAV2/8 Vector and Anti-VEGF Fab

In 1 animal (Animal C74440) in Group 2, elevated T-cell responses were observed at a single time point. In this animal, antibodies against anti-VEGF Fab and NAbs against AAV8 capsid were also observed. This animal lost expression of anti-VEGF Fab. In 1 animal (Animal C65873) in Group 5, displayed sustained T-cell responses to the pool B peptides of AAV8 capsid were observed including pre-injection baseline sample. The same animal had the highest levels of NAbs after administration of AAV2/8 vector. Another animal (Animal C68127) in the same group developed T-cells to all peptide pools of AAV8 capsid which were not sustained over time. This animal had antibodies against anti-VEGF Fab and the second highest level of NAbs. The animal lost expression of anti-VEGF Fab.

No other sustained T-cell responses to the transgene product were observed. No sustained T-cell responses were observed in animals in Group 6.

### Example 4-Animal Models Useful for Evaluating AAV2/8.aVEGF and Anti-VEGF Transgene Product

VEGF transgenic mice are used as animal models of Wet AMD. Two such models include the Rho/VEGF mouse model and the Tet/opsin/VEGF model.

### A. Rho/VEGF Mouse Model

Rho/VEGF mice are transgenic mice in which the rhodopsin promoter drives expression of human vascular endothelial growth factor (VEGF165) in photoreceptors, causing new vessels to sprout from the deep capillary bed of the retina and grow into the subretinal space, starting at postnatal Day 10. The production of VEGF is sustained and therefore the new vessels continue to grow and enlarge and form large nets in the subretinal space similar to those seen in humans with neovascular age-related macular degeneration. See Tobe, Takao, et al. “Evolution of neovascularization in mice with overexpression of vascular endothelial growth factor in photoreceptors.” Investigative ophthalmology & visual science 39.1 (1998): 180-188.

An enzyme-linked immunosorbent assay (ELISA) can be performed as follows. Briefly, plates are coated with 1 μg/mL of VEGF overnight at 4° C. 1% BSA is used as blocking buffer and is allowed to incubate at room temperature for 1 hour at 200 μL per well. Samples are loaded in duplicate at 100 μL per well and incubated for 1 hour at 37° C., followed by a second blocking buffer incubation. The primary antibody is a goat Anti-Human IgG H&L conjugated with Biotin which is left to incubate for 1 hour at room temperature at 100 μL per well. Secondary antibody is a 1:30,000 dilution of Streptavidin, loaded at 100 μL per well and incubated at room temperature for 1 hour. TMB solution is used as detection substrate (0.1M NaOAc Citric Buffer (pH 6.0), Hydrogen Peroxide, 100×TMB Stock), loaded at 150 μL per well and incubated at room temperature for 30 minutes without exposure to light. 50 μL of stop solution (2N H₂SO₄) is added to each well, and each plate was then read at 450 nm-540 nm.

In one study performed using this model and a test AAV8.aVEGF as described in the preceding examples, the ELISA results were as follows:

| | Dose | | | | | | |
|---|---|---|---|---|---|---|---|
| AAV8.aVEGF | (GC/eye) | Eye 1R | Eye 1L | Eye 2R | Eye 2L | Eye 3R | Eye 3L |
| Empty | 1.00E+10 | 0.00 | 0.00 | 0.00 | 0.00 | 0.00 | 0.00 |
| AAV8.aVEGF | 1.00E+08 | 0.00 | 0.00 | 0.00 | 0.00 | 0.00 | 313.58 |
| AAV8.aVEGF | 3.00E+08 | 0.00 | 0.00 | 0.00 | 0.00 | 0.00 | 0.00 |
| AAV8.aVEGF | 1.00E+09 | 0.00 | 530.45 | 0.00 | 0.00 | 324.01 | 0.00 |
| AAV8.aVEGF | 3.00E+09 | 0.00 | 0.00 | 0.00 | 0.00 | 208.71 | 0.00 |
| AAV8.aVEGF | 1.00E+10 | 232.23 | 239.19 | 139.30 | 0.00 | 0.00 | 0.00 |

| | Dose | | | | | | |
|---|---|---|---|---|---|---|---|
| | (GC/eye) | Eye 1R | Eye 1L | Eye 2R | Eye 2L | Eye 3R | Eye 3L |
| AAV8.aVEGF | 1.00E+10 | 0.00 | 0.00 | 0.00 | 0.00 | 0.00 | 0.00 |
| AAV8.aVEGF | 1.00E+08 | 0.00 | 0.00 | 0.00 | 0.00 | 0.00 | 313.58 |
| AAV8.aVEGF | 3.00E+08 | 0.00 | 0.00 | 0.00 | 0.00 | 0.00 | 0.00 |
| AAV8.aVEGF | 1.00E+09 | 0.00 | 530.45 | 0.00 | 0.00 | 324.01 | 0.00 |
| AAV8.aVEGF | 3.00E+09 | 0.00 | 0.00 | 0.00 | 0.00 | 208.71 | 0.00 |
| AAV8.aVEGF | 1.00E+10 | 232.23 | 239.19 | 139.30 | 0.00 | 0.00 | 0.00 |

| | Dose | | | | |
|---|---|---|---|---|---|
| Vector | (GC/eye) | Eye 4R | Eye 4L | Eye 5R | Eye 5L |
| Empty | 1.00E+10 | 0.00 | 0.00 | 0.00 | 0.00 |
| AAV8.aVEGF | 1.00E+08 | 0.00 | 251.56 | 0.00 | 0.00 |
| AAV8.aVEGF | 3.00E+08 | 0.00 | 0.00 | 0.00 | 0.00 |
| AAV8.aVEGF | 1.00E+09 | 0.00 | 564.75 | 0.00 | 0.00 |
| AAV8.aVEGF | 3.00E+09 | 355.31 | 207.95 | | |
| AAV8.aVEGF | 1.00E+10 | 134.53 | 0.00 | 214.05 | 167.79 |

Anti-VEGF FAb levels are shown in ng/eye.

### B. Tet/opsin/VEGF Mouse Model

Tet/opsin/VEGF mice are transgenic mice that are normal until given doxycycline in drinking water. Doxycycline induces very high photoreceptor expression of vascular endothelial growth factor (VEGF), leading to massive vascular leakage, culminating in total exudative retinal detachment in 80-90% of mice within 4 days of induction. See, Ohno-Matsui, Kyoko, et al. “Inducible expression of vascular endothelial growth factor in adult mice causes severe proliferative retinopathy and retinal detachment.” The American journal of pathology 160.2 (2002): 711-719.

The ELISA can be performed as described in Part A of this Example. In one study performed using this model and a test AAV8.aVEGF as described in the preceding examples, the ELISA results were as follows. Results are shown in the tables below as the average±standard deviation (Std).

| | | | Mouse Eye ID's | | | |
|---|---|---|---|---|---|---|
| | | Dose | 1R | 1L | 2R | 2L |
| Sample | Vector | (GC/eye) | Avg ± Std | Avg ± Std | Avg ± Std | Avg ± Std |
| Empty | Empty | 1.00E+10 | 0.00 ± 0.00 | 0.00 ± 0.00 | 0.00 ± 0.00 | 0.00 ± 0.00 |
| 1.00E+11 | AAV8.aVEGF | 1.00E+08 | 0.00 ± 0.00 | 216.34 ± 14.85 | 0.00 ± 0.00 | 0.00 ± 0.00 |
| 3.00E+11 | AAV8.aVEGF | 3.00E+08 | 88.23 ± 0.10 | 106.54 ± 1.01 | 0.00 ± 0.00 | 0.00 ± 0.00 |
| 1.00E+12 | AAV8.aVEGF | 1.00E+09 | 424.07 ± 19.26 | 0.00 ± 0.00 | 344.51 ± 30.67 | 0.00 ± 0.00 |
| 3.00E+12 | AAV8.aVEGF | 3.00E+09 | 581.28 ± 50.45 | 175.23 ± 20.45 | 254.13 ± 21.33 | 477.85 ± 34.54 |
| 1.00E+13 | AAV8.aVEGF | 1.00E+10 | 366.10 ± 20.76 | 309.06 ± 2.45 | 234.42 ± 4.78 | 173.46 ± 1.86 |

| | | | Mouse Eye ID's | | | | | |
|---|---|---|---|---|---|---|---|---|
| | | | 3R | 3R | 4R | 4L | 5R | 5L |
| | | Dose | Avg ± | Avg ± | Avg ± | Avg ± | Avg ± | Avg ± |
| Sample | Vector | (GC/eye) | Std | Std | Std | Std | Std | Std |
| Empty | Empty | 1.00E+10 | 0.00 ± 0.00 | 0.00 ± 0.00 | 0.00 ± 0.00 | 0.00 ± 0.00 | 0.00 ± 0.00 | 0.00 |
| | | | | | | | | 0.00 |
| 1.00E+11 | AAV8.aVEGF | 1.00E+08 | 0.00 ± 0.00 | 0.00 ± 0.00 | 0.00 ± 0.00 | 0.00 ± 0.00 | 0.00 ± 0.00 | 322.39 ± 23.43 |
| 3.00E+11 | AAV8.aVEGF | 3.00E+08 | 0.00 ± 0.00 | 0.00 ± 0.00 | 88.25 ± 1.96 | 0.00 ± 0.00 | 150.83 ± 26.63 | 444.96 ± 54.45 |
| 1.00E+12 | AAV8.aVEGF | 1.00E+09 | 0.00 ± 0.00 | 0.00 ± 0.00 | 537.61 ± 17.07 | 0.00 ± 0.00 | 0.00 ± 0.00 | 0.00 ± 0.00 |
| 3.00E+12 | AAV8.aVEGF | 3.00E+09 | 366.10 ± 23.68 | 285.85 ± 27.94 | 456.01 ± 27.81 | 155.71 ± 12.75 | 778.20 ± 143.25 | 270.15 ± 10.85 |
| 1.00E+13 | AAV8.aVEGF | 1.00E+10 | 498.95 ± 26.64 | 289.96 ± 8.95 | 291.74 ± 7.96 | 338.82 ± 6.03 | 165.10 ± 4.85 | 301.23 ± 21.10 |

### C. Other Animal Models

Other animal models of Wet AMD are utilized. In the laser trauma model, high-powered, focused laser energy is used to induce a break in Bruch's membrane. Subretinal injection of matrigel, VEGF, macrophages, lipid hydroperoxide, and/or polyethylene glycol induces choroidal neovascularization (CNV), a wet AMD pathology. See Pennesi, Mark E., Martha Neuringer, and Robert J. Courtney. “Animal models of age related macular degeneration.” Molecular aspects of medicine 33.4 (2012): 487-509.

Optimized rAAV.aVEGF vectors are generated, diluted and delivered into subretinal space of the transgenic mice eye with dosage described in the previous examples. Expressions of reporter genes, VEGF and anti-VEGF antibodies in the eye and/or plasma are determined by PCR, qPCR, ddPCR, oqPCR, Western Blot and ELISA as described in previous Examples. Electron Microscopy and Immunohistochemical analysis are also performed to evaluate the retinal neovascularization. The number of lesions per retina, area per lesion, neovascularization area per retina and traction retinal detachment Histopathological Evaluation of Retinas are quantified.

### Example 5-Assessment of Expression of Anti-VEGF Fab (Transgene Product) in Cynomolgus Monkeys

This study was conducted to assess the expression of the anti-VEGF Fab (transgene product) and to evaluate toxicity, immunogenicity, and biodistribution of an AAV8 vector expressing the anti-VEGF Fab following its administration in cynomolgus monkeys. In this report, expression of the transgene product and immunogenicity of the vector are described. Animals were administered an AAV2/8.aVEGF vector as described in these Examples or FFB-314 (control article) subretinally. Expression of transgene product in anterior chamber fluid and blood was determined by enzyme linked immunosorbent assay (ELISA). Immunogenicity was assessed by the presence of neutralizing antibodies (NAbs) against AAV8 capsid before and after administration. The transgene product is expressed in anterior chamber fluid of all animals administered the vector. The transgene product is not expressed in blood. Increase in levels of NAbs was observed in 1 animal (C73723) administered AAV8.aVEGF; this animal had pre-existing NAbs.

Animals in this study were administered a single dose of 1.00×10¹² genome copies (GC)/eye of AAV8.CB7.CI.aVEGFv3.rBG or formulation buffer, FFB-314.

AAV8.CB7.CI.aVEGFv3.RBG and FFB-314 were administered subretinally into the right eye (confirmed visually by appearance of a dome shaped retinal detachment/retinal bleb under microscope) in a total volume of 100 μL.

Animals were randomized using www.jamestease.co.uk/team-generator. One of 4 animals was selected using www.randomizer.org/by random and assigned to Group 2. The remaining 3 animals were assigned to Group 1. Group designation and dose levels for this study are presented in the following Table.

| | | Dose | | |
|---|---|---|---|---|
| Group | Treatment^{a} | (GC/eye) | No. of animals | Follow-up |
| 1 | AAV8.aVEGF | 1.00 × 10¹² | 1 M, 2 F | 7 days |
| 2 | FFB-314 | NA | 1 M | |

Abbreviation:
F = female;
GC = genome copies;
M = male;
NA = not applicable;
No. = number.
^{a}Test and control articles were administered subretinally into the right eye.

Animals were euthanized on Study day 7. Samples of anterior chamber fluid and blood were collected for determination of expression of the anti-VEGF FAb transgene product and/or the presence of NAbs against AAV8 capsid.

Subretinal injections were performed as described in the earlier examples. Collection of anterior chamber fluid was as described in earlier examples. For the ELISA, ninety-six-well, round-bottom plates were coated with 1 μg/mL of VEGF for expression of the anti-VEGF Fab transgene product, or 0.5 μg/mL of a commercial anti-VEGF Fab for expression of IgM and IgG against the Anti-VEGF Fab transgene product. The ELISA methods were as described in the earlier examples.

The following primary antibodies were used: 1.0 mg/mL Goat Anti-Human IgG H&L (Biotin) preadsorbed at a 1:10000 dilution in PBS; 0.5 mg/mL Goat Anti-Human IgG H&L (Biotin) preadsorbed at a 1:5000 dilution in PBS. The following secondary antibody was used: 1 mg/mL Streptavidin (HRP) at a 1:30000 dilution in PBS.

### Neutralizing Antibody Assay

Neutralizing antibody responses to AAV8 capsid were analyzed as follows. A Poly D lysine-coated 96-well black-walled/clear-bottom plate was seeded with human embryonic kidney 293 (HEK293) cells at 1×10⁵ cells/well (referred to as a cell plate); the plate was incubated at 37° C. overnight. The following day, the serum sample was heat-inactivated at 56° C. for 35 minutes. The heat-inactivated sample and a recombinant vector (AAV8.CMV.LacZ at 1×10⁹ GC/well; provided by the Penn Vector Core at the University of Pennsylvania) were used to formulate a serum-vector plate. The recombinant vector was diluted in serum-free Dulbecco's Modified Eagle Medium (DMEM) and incubated with 2-fold serial dilutions (starting at 1:5) of the heat inactivated samples at 37° C. for 1 hour. Prior to combining the serum vector plate with the cell plate, the HEK293 cells (now at 2×10⁵ cells/well) were infected with wild type HAdV5 (90 particles/cell) and incubated at 37° C. for 2 hours. After the incubation, the serum-vector plate and the cell plate were combined and incubated at 37° C. for 1 hour. Following the incubation, an equal volume of 20% fetal bovine serum (FBS) with DMEM was added to each well and the combined plate was incubated at 37° C. for additional 18 to 22 hours. The next day, the combined plate was washed with PBS and the HEK293 cells were lysed, and the lysate was developed using a mammalian β-galactosidase bioluminescence assay kit per the manufacturer's instructions. As a control, mouse serum was used instead of serum sample. The resulting luminescence was measured using a SpectraMax® M3 microplate luminometer. The resulting NAb titer was reported as the serum dilution that inhibits transduction of vector by at least 50% compared to the mouse serum.

### Statistical Analyses

Average and standard deviation values for concentrations of the Anti-VEGF Fab transgene product in anterior chamber fluid and blood were calculated using Microsoft Office Excel 2010.

### Results

### Expression of Anti-VEGF Fab Transgene Product in Anterior Chamber Fluid

The Anti-VEGF Fab transgene product was not expressed in anterior chamber fluid of the animal administered FFB-314. The anti-VEGF Fab transgene product was expressed in anterior chamber fluid collected from the right eye of all animals administered AAV8. aVEGF test vector. No expression was observed in the left eye. No difference in expression of the Anti-VEGF Fab transgene product between males and females was observed.

### Expression of Anti-VEGF Fab Transgene Product in Blood

In some patients administered a single IVT injection of Lucentis, ranibizumab was observed in serum (Xu, Invest Ophthalmol Vis Sci, 54:1616-24 (2013)). To determine if subretinal administration of AAV8.aVEGF test vector results in systemic exposure to the Anti-VEGF Fab transgene product, its concentrations were measured in serum.

Expression of the Anti-VEGF Fab transgene product was below nonspecific background levels in blood of the animal administered FFB-314 and all animals administered the AAV8.aVEGF vector as compared to matched pre-injection level.

### Presence of Neutralizing Antibodies Against AAV8 Capsid

Baseline levels of NAbs against AAV8 capsid were determined in serum from blood samples collected on Study day 0. The limit of detection was a 1:5 dilution; titers of <5 were considered undetectable.

| | |
|---|---|
| Animal | NAb titer |

| Treatment | Identification | Baseline | Study day 7 |
|---|---|---|---|
| FFB-314 | C64956 | 5 | 40 |
| AAV2/8.aVEGF | C73723 | 10 | 320 |
| 1.00 × 10¹² GC/eye | C74431 | <5 | <5 |
| | C65027 | 40 | 10 |

Abbreviations:
GC = genome copies;
NAb = neutralizing antibody.
Note:
the NAb titer values reported are the reciprocal dilutions of serum at which the relative luminescence units (RLUs) were reduced for 50% comparted to control wells (without sample). The limit of detection was 1:5 dilution of sample.

Animal administered FFB-314 had pre-existing NAbs against AAV8 capsid (see preceding Table). One animal (C74431) administered AAV8.aVEGF test vector did not have detectable NAbs to AAV8 capsid. In 2 animals (C73723, C65027) administered AAV8.aVEGF test vector, pre-existing NAbs against AAV8 capsid were observed, which persisted on Day 7 (see preceding Table).

Toxicity was evaluated based on clinical observations, body weights, indirect ophthalmoscopy, hematology, coagulation, clinical chemistry, and gross pathologic findings. There were no mortalities or unscheduled sacrifices in this study. No adverse clinical observations related to AAV8.aVEGF test vector or the Anti-VEGF Fab transgene product were noted for any animal. Several animals exhibited intermittent transient bouts of diarrhea with no impact to the welfare of the animals because body weights remained stable. No clinically meaningful changes in body weight during the study were observed for any animal. No adverse observations related to AAV8.aVEGF Test vector or the Anti-VEGF Fab transgene product were noted during indirect ophthalmoscopy in any animal. No clinically significant changes in hematology, coagulation, or clinical chemistry parameters were observed in any animal. All clinical pathology parameters were within normal ranges in all animals. There were no gross finding in animals C64956 and C74431. The surfaces of the right and left kidney of C73723 were pale. There was a focal lesion on the liver in C65027. In conclusion, there were no major toxicology findings.

The test vector in Examples 6-11 is rAAV8.CB7.CI.aVEGFrv3.rBG.

### Example 6-Expression of AAV2/8.aVEGF Vector in Cynomolgus Monkeys

This study was conducted to assess expression of the anti-VEGF transgene product and to evaluate toxicity, immunogenicity, and effect on normal retinal function of AAV2/8.aVEGF and the anti-VEGF transgene product, and shedding of AAV8.aVEGFin cynomolgus monkeys. The study is ongoing.

An AAV2/8.aVEGF described earlier in the examples is used this study. The vector is diluted in Dulbecco's phosphate-buffered saline (DPBS) with 0.001% Pluronic F-68. As a control article, FFB-314 (DPBS with 0.001% Pluronic F-68) was used. The study is ongoing. The results presented are based on the data collected at Month 3.

Macaques were used because they are the closest species to humans for studying retinal diseases. These monkeys and humans have similar eye anatomy, including fovea. The dimensions of the eyes are comparable, which allows determination of the human dose based on relative retinal areas.

Animals in this Example were administered a single dose of 1.00×10¹⁰ genome copies (GC)/eye of AAV8.aVEGF, or 1.00×10¹² GC/eye of AAV8.aVEGF, or FFB-314. AAV8.aVEGF and FFB-314 were administered subretinally into the right eye (confirmed visually by appearance of a dome-shaped retinal detachment/retinal bleb under microscope) in a total volume of 100 μL.

Animals were randomly assigned to 6 sets of 4 animals per set using www.jamestease.co.uk/team-generator. After assigning the sets, 1 of 4 animals from each of the 6 sets was selected using www.randomizer.org/at random and assigned to groups administered FFB-314 for each given administration date (Groups 2, 4, 6, 8, 10, and 12). The remaining 3 animals were assigned to groups administered 1×10¹² GC/eye or 1×10¹⁰ GC/eye AAV8.aVEGF (Groups 1, 3, 5, 7, 9, and 11). Group designation and dose levels for Examples 6 and 7 are presented below.

### Group Designation and Dose Levels

Group Designation and Dose Levels

| | | Dose | Number (#) | |
|---|---|---|---|---|
| Group | Treatment^{a} | (GC/eye) | of animals | Follow-up |
| 1 | AAV8.aVEGF test | 1.00 × 10¹² | 1 M, 2 F | 3 months |
| | vector | | | |
| 2 | FFB-314 | NA | 1 M | |
| 3 | AAV8.aVEGF test | 1.00 × 10¹⁰ | 2 M, 1 F | |
| | vector | | | |
| 4 | FFB-314 | NA | 1 M | |
| 5 | AAV8.aVEGF test | 1.00 × 10¹² | 2 M, 2 F^{b} | 1 year |
| | vector | | | |
| 6 | FFB-314 | NA | 1 F | |
| 7 | AAV8.aVEGF test | 1.00 × 10¹⁰ | 1 M, 2 F | |
| | vector | | | |
| 8 | FFB-314 | NA | 1 F | |
| 9 | AAV8.aVEGF test | 1.00 × 10¹² | 2 M, 1F | 7 days |
| | vector | | | |
| 10 | FFB-314 | NA | 1 M | |
| 11 | AAV8.aVEGF test | 1.00 × 10¹⁰ | 2 M, 1 F | |
| | vector | | | |
| 12 | FFB-314 | NA | 1 F | |

Abbreviation:
F = female;
GC = genome copies;
M = male;
NA = not applicable;
No. = number.
^{a}Test and control articles were administered subretinally into the right eye.
^{b}One female animal was euthanized during the study because of severe eye infection. The animal was replaced.

Samples of anterior chamber fluid and blood were collected for determination of expression of the Anti-VEGF Fab transgene product Subretinal injections were performed as described in earlier examples.

### A. Pharmacology

The results presented are based on the data collected at Month 3. In this report, expression of the anti-VEGF Fab transgene product is described.

### 1. Methods

Animals were administered AAV8.aVEGF test vector or FFB-314 (control article) subretinally. Expression of anti-VEGF transgene product in anterior chamber fluid and blood was determined by enzyme linked immunosorbent assay (ELISA) which was performed as described in previous examples.

### 2. Pharmacology Results

### (a) Expression of Transgene Product in Anterior Chamber Fluid:

The transgene product was not expressed in anterior chamber fluid of any animal administered FFB-314. The transgene product was expressed in anterior chamber fluid of all animals administered AAV8.aVEGF test vector. Onset of expression was rapid, generally within 7 days. Steady-state expression levels were achieved within 1 month. All animals continued to express the transgene product at steady-state levels until the last evaluated timepoint. However, overall expression levels of the anti-transgene product were greater in animals administered 1.00×10¹² GC/eye of AAV8.aVEGF test vector. No difference in expression of the transgene product between males and females was observed.

### (b) Expression of Transgene Product in Blood

In some patients administered a single IVT injection of Lucentis, ranibizumab was observed in serum (Xu, Invest Ophthalmol Vis Sci. 2013 Mar. 5, 54 (3):1616-24). To determine if subretinal administration of an AAV2/8.aVEGF test vector described in these examples results in systemic exposure to the anti-VEGF Fab transgene product, its concentrations were measured in serum. Expression of the anti-VEGF Fab transgene product was below nonspecific background levels in blood of all animals administered AAV8.aVEGF test vector compared to matched pre-injection levels.

### 3. Conclusion:

Anti-VEGF Fab transgene product is expressed in anterior chamber fluid of all animals administered AAV8.aVEGF test vector.

Anti-VEGF Fab transgene product is not expressed in blood of any animal administered AAV8.aVEGF test vector.

### B. Toxicology

In this report, evaluation of toxicity of an AAV2/8.aVEGF test vector is described. Animals were administered AAV8.aVEGF test vector or FFB-314 (control article) subretinally. Toxicity was evaluated based on clinical observations, body weights, ocular pressure, indirect ophthalmoscopy, spectral domain optical coherence tomography, hematology, coagulation, clinical chemistry, and gross pathologic findings, and histopathologic findings.

Ocular pressure was evaluated via rebound tonometry (Tono Vet). This method is easy to use and does not require topical anesthesia. Rebound tonometry estimates OP by using an induction coil to magnetize a small, plastic-tipped metal probe that is launched against the cornea. As the probe rebounds back to the instrument, it creates an induction current from which the OP is calculated. Up to 2 readings were taken, from which an average OP was determined, and accuracy of the results was indicated. Application with the device was performed according to the manufacturer's instructions.

Retinal structure (at a micron-level resolution) was evaluated by in vivo, non-invasive, cross-sectional retinal microscopy with SD-OCT (Spectralis OCT, Heidelberg Engineering, Carlsbad, CA). Pupils were dilated with phenylephrine 2.5% and tropicamide 1%. En-face retinal imaging was performed with near infrared (NIR) reflectance (REF) and in a subset of animals with NIR fundus autofluorescence (FAF) using the scanning laser ophthalmoscope of this imaging system. Spectral domain optical coherence tomography scanning was performed with 9 mm long horizontal and vertical cross-sections through the fovea and overlapping 30×25 mm raster scans extending into the near midperiphery.

The only adverse AAV8.aVEGF test vector-related finding was significant retinal thinning and loss of photoreceptors observed by spectral domain optical coherence tomography in animals administered 1.00×10¹² GC/eye of test vector.

### C. Electroretinogram (ERG)

In this subpart, assessment of effects of AAV8.aVEGF test vector and the anti-VEGF Fab transgene product on normal retinal function is described. Animals were administered AAV8.aVEGF test vector or FFB-314 (control article) subretinally. Retinal function was evaluated by the full-field electroretinogram (ERG). The full-field ERG is a widely used electrophysiologic test of retinal function. Electroretinogram is a mass electrical potential generated by the retina in response to light stimulus. Usually, it is recorded by an electrode in contact with the corneal surface. Electroretinograms in this study were conducted in accord with the recommendations set by the International Society for Clinical Electrophysiology of Vision (ISCEV; McCulloch, Doc Ophthalmol. 2015 February; 130 (1): 1-12. 2015). The results presented are based on the data collected at Month 3. In this report, assessment of effects of AAV8.aVEGF test vector and the anti-VEGF Fab transgene product on normal retinal function is described. Animals were administered AAV8.aVEGF test vector or FFB-314 (control article) subretinally. Retinal function was evaluated by the full-field electroretinogram. In summary, administration of 1.00×10¹⁰ genome copies (GC)/eye of AAV8.aVEGF test vector do not impair retinal function. In contrast, administration of 1.00×10¹² GC/eye of AAV8.aVEGF test vector impairs retinal function.

### 1. Electroretinogram (ERG) Parameters

An electroretinogram (ERG) generated usually when all retinal cells are active respond to a flash stimulation (a dark-adapted animal, moderate to intense flash). The 2 components are the following:
a-wave: cornea-negative signal, first after the flash. Origin: photoreceptor photocurrent, the most direct signature of photoreceptor function.
b-wave: cornea-positive signal following the a-wave generated mostly by on-bipolar cells (second order neurons downstream from photoreceptors).

In this study, the following International Society for Clinical Electrophysiology of Vision (ISCEV) standard and additional protocols were used:
Dark-adapted rod ERG: Stimulus intensity: 0.01 to 0.02 cd s m⁻². Response: b-wave only, no a-wave. Source: rod “on” bipolar cells (second order neurons driven by input from rods). Meaning: a measure of rod function. Designation in data sheets: “Dim flash”.
Dark-adapted standard flash ERG: Stimulus intensity: 3 cd s m⁻². Response: combined rod-cone a- and b-waves; 60% to 70% of the signal being generated by the rod-driven pathway. Source: photoreceptors, both rods and cones (a-wave); higher order neurons driven by both rods and cones. Meaning: a measure of mostly rod function; less sensitive to the state of dark adaptation and less variable than the “dim flash” response. Designation in data sheets: “Standard flash”.
Dark-adapted bright flash ERG: Stimulus intensity: 10 cd s m⁻². Response and meaning: same as for the “standard flash” response, but bright flash response is larger in magnitude and may be less variable. Designation in data sheets: “Bright flash”.
Light-adapted standard flash cone ERG: Stimulus intensity: 3 cd s m⁻², delivered in presence of 30 cd m⁻² background light after 5 minutes of light adaptation. Response: a- and b-waves generated by cone-driven pathways. Meaning: in presence of background light which completely desensitizes rods the ERG is produced exclusively by cones and cone-driven secondary retinal neurons and is a measure of the cone function. Designation in data sheets: “Standard cone ERG”.
Light-adapted bright flash cone ERG (in addition to the ISCEV standard): Stimulus intensity: 10 cd s m⁻², delivered in presence of 30 cd m⁻² background light after 5 minutes of light adaptation. Response and meaning: cone-driven ERG as in case of the “Standard cone ERG”, but of greater magnitude and potentially less variable.

ERG measures (a-wave amplitude, a-wave implicit time, b-wave amplitude, b-wave implicit time) were summarized using mean and standard deviation (SD) for treated eyes and control eye, and for each treatment (FFB-314 (vehicle) groups, AAV8.aVEGF test vector 1.00×10¹⁰ GC/eye groups, AAV8.aVEGF test vector 1.00×10¹² GC/eye groups). The paired t-test was used for comparing the ERG measures between AAV8.aVEGF test vector (treated) eye and FFB-314 (control eye), and for comparison between post-injection vs. pre-injection. The two-sample t-test was used for comparing the ERG measures between AAV8.aVEGF test vector 1.00×10¹⁰ GC/eye groups vs. FFB-314 (vehicle) groups, AAV8.aVEGF test vector 1.00×10¹² GC/eye groups vs. FFB-314 (vehicle) groups, and AAV8.aVEGF test vector 1.00×10¹² GC/eye groups vs. AAV8.aVEGF test vector 1.00×10¹⁰ GC/eye groups. The t-test is appropriate even when the sample size is small [Winter JCF. Using the Student's t-test with extremely small sample sizes. Practical Assessment, Research and Evaluation. 2013; 18 (10). Available online: pareonline.net/-getvn.asp?v=18&n=10] 1 or the data are not normally distributed. See, Shuster JJ. Diagnostic for assumptions in moderate to large simple clinical trials: do they really help? Statist. Med. 2005; 24:2431-2438; Ganju J. D. Comment on “Diagnostic for assumptions in moderate to large simple clinical trials: do they really help?” Statist. Med. 2006; 25:1798-1800.] All the statistical analyses were performed in SAS v9.4 (SAS Institute Inc., Cary, NC), and two-sided p-value≤0.05 is considered as statistically significant.

### 2. Results

Anti-VEGF Fab transgene product was expressed in all animals administered AAV8.aVEGF test vector (see pharmacology results in Part A of this Example). Retinal function 3 months following administration of AAV8.aVEGF test vector or FFB-314 (post-injection) was compared to retinal function before administration (pre-injection) for treated and untreated eyes. An animal in Group 8 was excluded from data analyses due to an unobtainable ERG following administration of FFB-314.

| | | | | | | p-value | | |
|---|---|---|---|---|---|---|---|---|
| | | | | | | Low | High | High |
| | | | | Low | High | dose | dose | dose |
| | | Stimulus | FFB-314 | dose | dose | vs. | vs. | vs. |
| ERG | | intensity | Mean | Mean | Mean | FFB- | FFB- | Low |
| test | Parameter | (cd · s · m⁻²) | (SD) | (SD) | (SD) | 314 | 314 | dose |
| Dark- | a-wave | 3 | 68.2 | 58.4 | 31.3 | 0.4 | 0.003 | 0.01 |
| adapted | amplitude | | (10.0) | (17.4) | (12.4) | | | |
| | (uv) | 10 | 113.8 | 109.8 | 54.1 | 0.8 | 0.002 | 0.002 |
| | | | (0.9) | (25.6) | (21.0) | | | |
| Light- | a-wave | 3 | 19.9 | 18.7 | 9.8 | 0.65 | 0.007 | 0.002 |
| adapted | amplitude | | (4.1) | (3.5) | (3.7) | | | |
| | (uv) | 10 | 36.1 | 33.3 | 19.5 | 0.61 | 0.02 | 0.01 |
| | | | (7.1) | (7.4) | (8.6) | | | |

Abbreviations: ERG = electroretinogram; GC = genome copies; SD = standard deviation.
Low dose: 1.00 × 10¹⁰ GC/eye of AAV8.aVEGF test vector
High dose: 1.00 × 10¹² GC/eye of AAV8.aVEGF test vector.

a. Comparison of Retinal Function Between Treatment Groups

For treated eyes, retinal function post-injection was comparable between animals in low-dose group (1.00×10¹⁰ GC/eye of AAV8.aVEGF test vector) and FFB-314 group (see preceding Table). For treated eyes, retinal function post-injection in animals in high-dose group (1.00×10¹² GC/eye of AAV8.aVEGF test vector) was significantly reduced compared to animals in FFB-314 group (see preceding Table). For treated eyes, retinal function post-injection in animals in high-dose group was significantly reduced compared to animals in low-dose group (see preceding Table). For untreated eyes, retinal function post-injection was comparable to pre-injection for all groups.

b. Comparison of Retinal Function within Treatment Groups

For treated eyes, in animals in low-dose and FFB-314 groups, retinal function post-injection was comparable to matched pre-injection baseline. For treated eyes, in animals in high-dose group, retinal function post-injection was significantly reduced compared to matched pre-injection baseline. For untreated eye, retinal function post-injection was comparable to matched pre-injection baseline.

### E. Virus Shedding

Shedding of AAV8.aVEGF test vector was determined by quantitative PCR analysis targeting transgene-specific sequence in samples of tears, nasal secretion, serum, saliva, urine, and feces. Samples were collected before and after administration of AAV8.aVEGF test vector or FFB-314. AAV8.aVEGF test vector DNA was readily detectable in most samples collected from animals administered AAV8.aVEGF test vector. The presence of AAV8.aVEGF DNA was dose-dependent, transient, and decreased over time.

### F. Immunogenicity

In this study, immunogenicity of AAV8.aVEGF test vector and the anti-VEGF Fab transgene product is described. Immunogenicity was assessed by the following:
The presence of IgM and IgG antibodies against the anti-VEGF Fab transgene product using enzyme linked immunosorbent assay (ELISA);
The presence of neutralizing antibodies (NAbs) against AAV8 capsid using NAb assay;
T-cell responses to AAV8.aVEGF test vector and the anti-VEGF Fab transgene product using enzyme linked immunospot (ELISPOT) assay.

Animals were administered AAV8.aVEGF test vector or FFB-314 (control article) subretinally as described earlier in this Example. No sustained IgM, IgG, or T-cell responses to the anti-VEGF Fab transgene product were observed in any animal. Animals administered 1.00×10¹² GC/eye AAV8.aVEGF test vector developed a higher neutralizing antibody (Nab) response to AAV8 capsid than animals administered 1.00× 10¹⁰ GC/eye AAV8.aVEGF test vector. The NAb response was higher in animals with pre-existing NAbs. Slightly increased T-cell responses against AAV8 capsid were observed in 2 of 6 animals administered 1.00×10¹² GC/eye test vector.

### Results

Anti-VEGF Fab transgene product was expressed in all animals administered AAV8.aVEGF test vector (Example 6). There was no significant IgM against the Anti-VEGF Fab transgene product in serum or in anterior chamber fluid of animals administered FFB-314. IgG against the Anti-VEGF Fab transgene product above baseline level was not observed in animals administered FFB-314.

IgM against the anti-VEGF Fab transgene product was elevated above the baseline level in anterior chamber fluid of 1 animal administered 1.00×10¹⁰ GC/eye of AAV8.aVEGF test vector. However, as there was no corresponding elevation in the serum, therefore this observation was not clinically significant. IgG against the anti-VEGF Fab transgene product above baseline level was not observed in this treatment group.

IgM against the anti-VEGF Fab transgene product above baseline level was observed in anterior chamber fluid of 1 animal administered 1.00×10¹² GC/eye of AAV8.aVEGF test vector. However, as there was no corresponding elevation in the serum, this observation was not clinically meaningful. IgG against the anti-VEGF Fab transgene product above baseline level was observed in serum and anterior chamber fluid from another animal and in anterior chamber fluid only of a third animal in this treatment group. However, as neither was preceded by any detectable IgM, these observations were not clinically meaningful. The presence of IgG in these animals was not associated with loss of expression of the anti-VEGF Fab transgene product.

Baseline levels of NAbs against AAV8 capsid were determined in serum from blood samples collected on Study day 0. The limit of detection was a 1:5 dilution; titers of <5 were considered undetectable.

In 4 of 6 animals administered FFB-314, pre-existing NAbs were not observed. Two animals that were followed by Study day 90 did not develop NAbs. In 2 animals administered FFB-314, pre-existing NAbs were observed. The levels of NAbs in these 2 animals fluctuated no more than 2 two-fold serial dilutions during the study.

In 2 of 9 animals administered 1.00×10¹⁰ GC/eye of AAV8.aVEGF test vector, pre-existing NAbs were not observed. In 1 animal that was followed by Study day 90, NAbs were not observed following administration of AAV8.aVEGF test vector. In animals with pre-existing NAbs, their levels increased by no more than 4 two-fold serial dilutions following administration of AAV8.aVEGF test vector.

In 4 of 9 animals administered 1.00×10¹² GC/eye of AAV8.aVEGF test vector, pre-existing NAbs were not observed. Regardless of status of pre-existing NAbs, in most animals, an increase in NAb response of up to 9 two-fold serial dilutions was observed following administration of AAV8.aVEGF. This response was sustained through the Study day 90.

T-cell responses to AAV8.aVEGF test vector were observed in 1 animal administered FFB-314 at a single timepoint. In 1 animal, non-specific T cell responses were observed at all timepoints.

Sustained T-cell responses to AAV8.aVEGF test vector were not observed in animals administered 1.00×10¹⁰ GC/eye of test vector.

In 4 of 6 animals administered 1.00×10¹² GC/eye of AAV8.aVEGF test vector, low-level immune response against AAV8.aVEGF test vector was observed. In 2 of 4 animals with low-level immune response, a sustained (more than 2 consecutive time points) response was observed. Sustained T-cell responses to the anti-VEGF Fab transgene product were not observed in any animal.

No sustained IgM, IgG, or T-cell responses to the Anti-VEGF Fab transgene product were observed in any animal.

Animals administered 1.00×10¹² GC/eye AAV8.aVEGF test vector developed a higher NAb response to AAV8.aVEGF test vector than animals administered 1.00×10¹⁰ GC/eye of the same test vector. The NAb response was higher in animals with pre-existing NAbs. Slightly increased T-cell responses against this AAV8.aVEGF test vector were observed in 2 of 6 animals administered 1.00×10¹² GC/eye the AAV8.aVEGF test vector.

### Example 7-Evaluation of Distribution of AAV2/8 Vector mRNA and Anti-VEFG Fragment Antigen-Binding Following Subretinal Administration of AAV2/8 Vectors in Cynomolgus Monkeys

This study was conducted to evaluate retinal distribution of AAV2/8 vector mRNA and distribution of anti-VEGF Fab throughout the eye following subretinal administration of AAV2/8 vector utilizing tissues from Example 3, Example 5 and Example 6. Levels of mRNA in different parts of retina were assessed by quantitative reverse transcription-polymerase chain reaction and by in situ hybridization. Concentrations of anti-VEGF Fab were determined in retinal sections, anterior chamber fluid and vitreous humor by enzyme-linked immunosorbent assay.

mRNA for AAV2/8 vector is distributed throughout the entire retina following subretinal administration. Similarly, anti-VEGF Fab is distributed throughout the entire retina and is detected in both, vitreous and anterior chamber fluid.

Vector

AAV2/8.UbC.PI.aVEGFv2.SV40
AAV2/8.UbC.PI.aVEGFv3.SV40
AAV2/8.CB7.CI.aVEGFv2.rBG
AAV2/8.CB7.CI.aVEGFv3.rBG

Site of subretinal administration is denoted by a retinal bleb, which can be visualized by SD OCT. In all SD OCT images, retinal blebs are visible.

Levels of mRNA for AAV2/8.aVEGF Test Vector in Retina Determined by RT-qPCR

mRNA for AAV8.aVEGF test vector was not detected in the retina of the animal administered FFB-314. mRNA for the AAV8.aVEGF test vector was detected in retinas of all animals administered the AAV8.aVEGF test vector. The highest level of mRNA was detected in the retinal sections that incorporated the site of the subretinal injection. However, mRNA for the AAV8.aVEGF test vector was also detected in sections outside of the injection bleb. mRNA levels in these sections were lower than those in the bleb. The levels were up to 4 logs lower in sections most peripheral to the injection blebs. In sections immediately adjacent to the injection bleb, the levels of mRNA were intermediate.

Expression of mRNA for AAV2/8 Vectors in Retina Determined by In Situ Hybridization (ISH)

Expression of mRNA for the AAV2/8 vector determined by ISH was high at the injection site. The transduced cells within retinal layers included RPE cells, photoreceptors, and ganglion cells. Expression of mRNA was lower when moving away from the injection site, disappearing almost completely in the areas most distal to the injection site.

### Concentrations of Anti-VEGF Fab in Anterior Chamber Fluid, Vitreous, and Retina

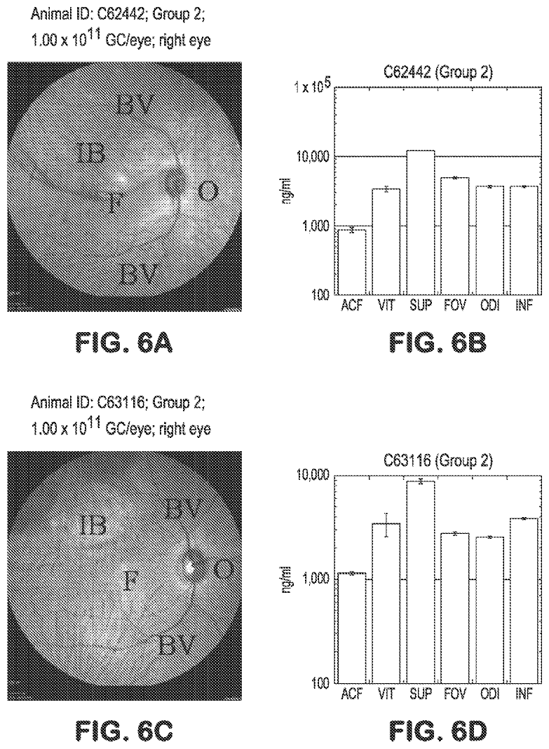

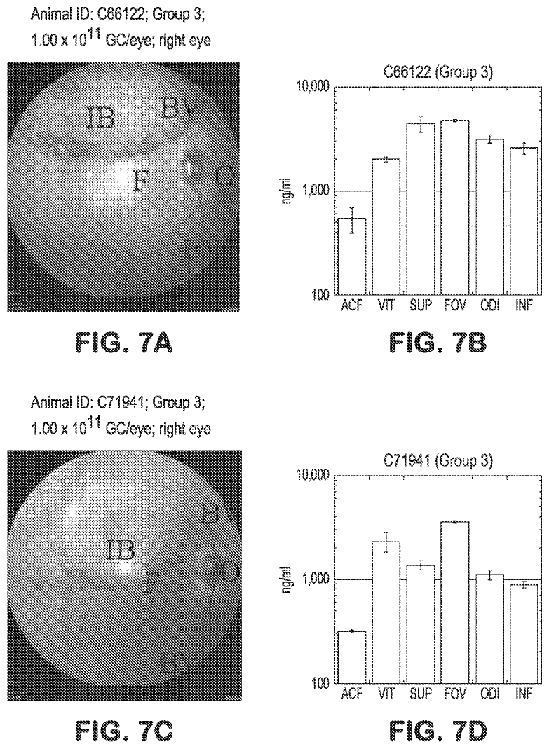

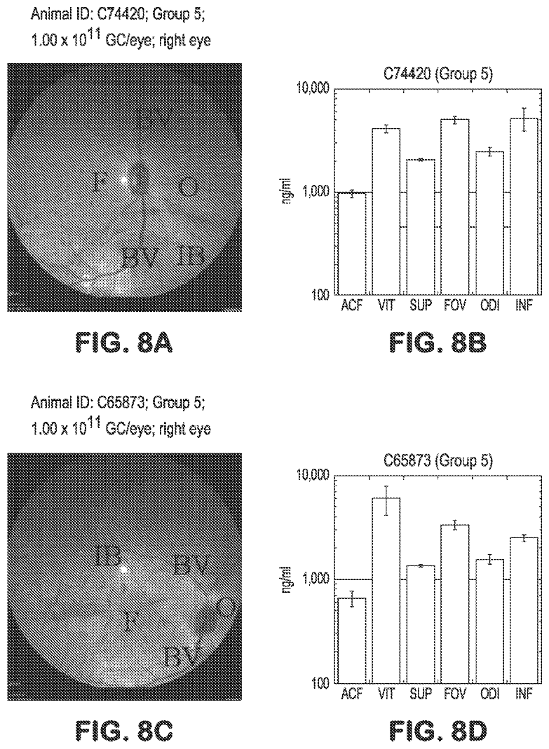

Anti-VEGF Fab was expressed in retinas, vitreous, and anterior chamber fluid of eyes of all animals administered AAV2/8 vector (FIGS. 6-8). Expression in the vitreous was 3- to 9-fold higher than in the anterior chamber fluid. With the exception of 1 animal (C65873) in Group 5 (FIG. 8), maximal expression in the retinal segments was 1.2- to 3.6-fold higher than in the vitreous. This concentration gradient is likely a reflection of the mechanism of distribution of anti-VEGF Fab. Anti-VEGF Fab is secreted into vitreous by transduced retina and then diffuses form vitreous to the anterior chamber fluid. Of note, expression of anti-VEGF Fab throughout the retina is more uniform than expression of mRNA.

Overall, functional AAV2/8 vector is surprisingly distributed throughout the entire retina following subretinal administration as evidenced by the expression of the vector mRNA by the transduced cells, instead of being limited to the injection bleb. Anti-VEGF Fab is also surprisingly distributed throughout the entire retina including retinal segments that are peripheral to the injection bleb, and is detected in both, vitreous and anterior chamber fluid.

### Example 8—Determination of Affinity for Binding OF Anti-VEGF Transgene Product to Recombinant Human VEGF

This study was conducted to determine affinity for binding of the Anti-VEGF Fab heavy and line chains product to recombinant human VEGF. Binding affinity was determined using Biacore 3000 system, based on surface plasmon resonance (SPR) technique. This technique is based on the plane-polarized light hitting a sensor chip under the conditions of total internal reflection. Interaction between immobilized ligands (e.g., VEGF) and interacting molecules (e.g., Anti-VEGF Fab transgene product) on the sensor chip causes a change in angle of reflectivity of plane-polarized light. This change is immediately detected by sensogram in real time as response units (Daghestani, Theory and applications of surface plasmon resonance, resonant mirror, resonant waveguide grating, and dual polarization interferometry biosensors. Sensors (Basel). 2010; 10 (11): 9630-46.). The equilibrium binding affinity constant for binding of the Anti-VEGF Fab transgene product is consistent with published range for ranibizumab.

### Example 9: Tissue Cross-Reactivity Study

The objective of this study was to assess using immunohistochemical techniques, the potential cross-reactivity of the Sponsor supplied antibody Fab fragment aVEGF transgene product with histologically prepared cryo-sections from a selected panel of human tissues.

Anti-VEGF Fab transgene product (1 mg/mL) (“Test product”) and ranibizumab (0.97 mg/mL) were used for this study. Natural Human IgG Fab Fragment Protein (the “Control Article”) was supplied at a protein concentration of 14.64 mg/mL. To facilitate immunohistochemical detection the Test transgene product, natural human IgG Fab fragment protein and ranibizumab were conjugated with biotin. The respective protein concentrations were 2.79 mg/mL, 2.88 mg/mL and 2.89 mg/mL. Cryo-sections from the control material and the human tissues for examination were prepared. The assessment of tissue viability indicated that the panel of human tissues was viable. Following slide evaluation of the control titration the following three concentrations of Test transgene product-Biotin: 5, 2.5 and 1.25 μg/mL, and the following concentration of ranibizumab-Biotin: 2.5 μg/mL, were selected for use in the tissue titration. In the tissue titration no specific positive staining was observed with anti-VEGF transgene product-Biotin or ranibizumab-Biotin in any of the tissues examined. All other observed staining was variable and considered to be non-specific.

Under the conditions of this study, antigen-specific binding of Test transgene product-Biotin and ranibizumab-Biotin was demonstrated in the positive control materials (human glioblastoma and VEGF protein spots). No similar staining was observed with Natural human IgG Fab fragment protein-Biotin or the antibody diluent at the concentrations examined in the tissue titration.

### Example 10—Clinical Study

A rAAV8.aVEGF vector was selected for further study which provides the advantage of single sub-retinal administration, thereby reducing the burden of repeated injections. Continued expression of anti-VEGF Fab in NHP for over 6 months and reduction in neovascularization in an animal model of WAMD treated with an rAAV8.aVEGF vector have been demonstrated in pre-clinical studies, and safety of sub-retinal injection is evaluated in non-human primates. The initial clinical study evaluates the safety and transgene expression after a single sub-retinal injection of an rAAV8.aVEGF test vector as described above. Once injected sub-retinally, these vectors are expected to continue to release an anti-VEGF Fab transgene product and block the angiogenic signal thereby protecting the retina from further damage.

Each dosing cohort includes 3 subjects. The first 3 subjects enrolled start with the lowest dose and each group escalates. After each first rAAV8.aVEGF dose, there is a 4 week observation period for safety prior to the next patient being dosed. The primary safety endpoint is at 6 weeks post administration of rAAV8.aVEGF.

### Primary Endpoints

Ocular and non-ocular safety assessment at 6 weeks, 24 weeks, 6 and 12 months post procedure.

### Secondary Endpoints

Ocular and non-ocular safety over 106 weeks
Mean change from baseline in aqueous rAAV8.aVEGF protein over time
Mean change from baseline in BCVA over time
Proportion of subjects gaining or losing≥15 letters compared to baseline as per BCVA at Week 26, Week 54, and Week 106.
Mean change from baseline in CRT as measured by SD-OCT over time
Mean number of ranibizumab rescue injections over time
Time to 1st rescue ranibizumab injection
Mean change from baseline in CNV and lesion size and leakage area based on FA over time
Immunogenicity measurements (NAb to AAV8, binding antibodies to AAV8, antibodies to aVEGF protein, and Enzyme-Linked ImmunoSpot [ELISpot]).
Vector shedding analysis in serum and urine.
   Exploratory endpoints:
Mean change from baseline over time in area geographic atrophy per fundus autoflorescence (FAF) Incidence of new area of geographic atrophy by FAF (in subjects with no geographic atrophy at baseline).
Proportion of subjects gaining or losing ≥ and ≥10 letters, respectively, compared with baseline as per BCVA.
Proportion of subjects who have a reduction of 50% in rescue injections compared with previous year
Proportion of subjects with no fluid on SD-OCT

For the present study, patients must have a diagnosis of neovascular age-related macular degeneration (wet AMD) and meet the following criteria.

### Inclusion Criteria:

In order to be eligible to participate in this study, a subject must meet all of the following criteria. It is understood that one or more of these criteria may not be required for further studies and for treatment of other populations.
1. Males or females aged 50 years or above.
2. Sentinel subject for each dose cohort must have a BCVA≤20/100 and ≥20/400 (≤65 and ≥35 ETDRS letters) in the study eye.
   a. Following the sentinel subject evaluation, the rest of the subjects in the dose cohort must have a BCVA between ≤20/63 and ≥20/400 (≤75 and ≥35 ETDRS letters).
3. In the case both eyes are eligible, study eye must be the subject's worse-seeing eye, as determined by the Investigator.
4. Must have a documented diagnosis of subfoveal CNV secondary to AMD in the study eye.
   a. CNV lesion characteristics: lesion size needs to be less than 10 disc areas (typical disc area is 2.54 mm2), blood and/or scar <50% of the lesion size.
5. Must have received at least 4 intravitreal injections of an anti-VEGF agent for treatment of nAMD in the study eye in the 8 months (or less) prior to Visit 1, with anatomical response documented on SD-OCT.
6. Must have subretinal or intraretinal fluid present at Visit 1 in the study eye, evidenced on SD-OCT.
7. Must be pseudophakic (status post cataract surgery) in the study eye.
8. Must be willing and able to comply with all study procedures and be available for the duration of the study.
9. Females of childbearing potential must have a negative urine pregnancy test at the screening visit, have negative serum results by Day 8, and be willing to have additional pregnancy tests during the study.
10. Sexually active subjects (both female and male) must be willing to use a medically accepted method of barrier contraception (e.g., condom, diaphragm, or abstinence) from screening visit until 24 weeks after vector administration. Cessation of birth control after this point should be discussed with a responsible physician.
11. Must be willing and able to provide written, signed informed consent.

### Exclusion Criteria:

Subjects who meet any of the following exclusion criteria are not eligible to participate in the study. It is understood that future studies and treatment of other patient populations may not include any or all of these criteria.
1. CNV or macular edema in the study eye secondary to any causes other than AMD.
2. Blood occupying ≥50% of the AMD lesion or blood >1.0 mm² underlying the fovea in the study eye.
3. Any condition preventing VA improvement in the study eye, eg, fibrosis, atrophy, or retinal epithelial tear in the center of the fovea.
4. Active or history of retinal detachment in the study eye.
5. Advanced glaucoma in the study eye.
6. Any condition in the study eye that, in the opinion of the Investigator, may increase the risk to the subject, require either medical or surgical intervention during the course of the study to prevent or treat vision loss, or interfere with study procedures or assessments.
7. History of intraocular surgery in the study eye within 12 weeks prior to the screening visit. Yttrium aluminum garnet capsulotomy is permitted if performed >10 weeks prior to the screening visit.
8. History of intravitreal therapy in the study eye, such as intravitreal steroid injection or investigational product, other than anti-VEGF therapy, in the 6 months prior to screening
9. Presence of an implant in the study eye at screening (excluding intraocular lens).
10. History of malignancy requiring chemotherapy and/or radiation in the 5 years prior to screening. Localized basal cell carcinoma is permitted.
11. Receipt of any investigational product within the 30 days of enrollment or 5 half-lives of the investigational product, whichever is longer.
12. Participation in any other gene therapy study.
13. History of therapy known to have caused retinal toxicity, or concominant therapy with any drug that may affect visual acuit or with known retinal toxicity, eg, chloroquine or hydroxychloroquine.
14. Ocular or periocular infection in the study eye that may interfere with the surgical procedure.
15. Myocardial infarction, cerebrovascular accident, or transient ischemic attacks within the past 6 months.
16. Uncontrolled hypertension (systolic blood pressure [BP]>180 mmHg, diastolic BP>100 mmHg) despite maximal medical treatment.
17. Any concomitant treatment that, in the opinion of the Investigator, may interfere with ocular surgical procedure or healing process.
18. Known hypersensitivity to ranibizumab or any of its components or past hypersensitivity (in the Investigator's opinion) to agents like rAAV8.aVEFG test vector.
19. Any serious or unstable medical or psychological condition that, in the opinion of the Investigator, would compromise the subject's safety or successful participation in the study.
   Criteria for Continuing Study after Receiving Ranibizumab

At Visit 2, subjects are assessed for initial anti-VEGF response to ranibizumab. Subjects undergo both SD-OCT and BCVA, which are compared by the Investigator with the Visit 1 values:
1. Responsive (subjects continue in the study): Response is defined as reduction in CRT >50 microns or >30% improvement in fluid by SD-OCT.
2. Non-responsive (subjects exit the study as early withdrawals): Non-response is defined as not meeting the criteria above. Additional subjects continue to be enrolled until up to 6 subjects in each cohort receive a single dose of rAAV8.aVEFG test vector.

At this visit central lab results are reviewed. Any subjects with the following values are withdrawn:
3. Aspartate aminotransferase (AST)/alanine aminotransferase (ALT)>2.5× upper limit of normal (ULN)
4. Total bilirubin>1.5×ULN unless the subject has a previously known history of Gilbert's syndrome and a fractionated bilirubin that shows conjugated bilirubin<35% of total bilirubin
5. Prothrombin time (PT)>1.5×ULN
6. Hemoglobin<10 g/dL for male subjects and <9 g/dL for female subjects
7. Platelets<100×103/μL
8. Estimated glomerular filtration rate (GFR)<30 mL/min/1.73 m²

In the initial study, ranibizumab (LUCENTIS, Genentech) 0.5 mg is administered by intravitreal injection on Visit 1, 14 days prior to rAAV8.aVEGF test vector subretinal delivery. The rAAV8.aVEGF is given by subretinal administration by a retinal surgeon under local anesthesia. The procedure involves standard 3 port pars plana vitrectomy with a core vitrectomy followed by a single subretinal administration into the subretinal space by a subretinal cannula (36 to 41 gauge). 100-150 microliters of rAAV8.aVEGF is delivered. Patients receive one of 3, 4 or 5 doses. Three dose levels: 3×10⁹ genome copies (GC)/eye, 1×10¹⁰ GC/eye, and 6×10¹⁰ GC/eye. Starting at 4 weeks post-rAAV8.aVEGF test vector administration, the subject may receive intravitreal ranibizumab rescue therapy in the study eye for disease activity if 1 or more of the following rescue criteria apply: Vision loss of ≥5 letters (per Best Corrected Visual Acuity [BCVA]) associated with accumulation of retinal fluid on Spectral Domain Optical Coherence Tomography (SD-OCT). Choroidal neovascularization (CNV)-related increased, new, or persistent subretinal or intraretinal fluid on SD-OCT. New ocular hemorrhage.

Further rescue injections may be deferred per the clinician's discretion if one of the following sets of findings occur: Visual acuity is 20/20 or better and central retinal thickness is “normal” as assessed by SD-OCT, or Visual acuity and SD-OCT are stable after 2 consecutive injections. If injections are deferred, they are resumed if visual acuity or SD-OCT get worse per the criteria above.

### Example 11—Dose Escalation Study

This Phase I, open-label, multiple-cohort, dose-escalation study is designed to evaluate the safety and tolerability of rAAV8.aVEGF gene therapy in subjects with previously treated neovascular AMD (nAMD). Three doses are studied in approximately 18 subjects. Subjects who meet the inclusion/exclusion criteria and have an anatomic response to an initial anti VEGF injection receive a single dose of rAAV8.aVEGF administered by subretinal delivery. rAAV8.aVEGF uses an AAV8 vector that contains a gene that encodes for a monoclonal antibody fragment which binds to and neutralizes VEGF activity. Safety is the primary focus for the initial 24 weeks after rAAV8.aVEGF administration (primary study period). In certain embodiments, the study includes administering an anti-VEGF antibody, e.g., ranibizumab, and response is measured at week 1 (Visit 2) by SD-OCT. For patients responsive to this treatment, rAAV8.aVEGF may be administered at Visit 3 (week 2), post-anti-VEGF antibody administration and safety is then assessed through week 26 (24 weeks post-rAAV8.aVEGF administration). Following completion of the primary study period, subjects continue to be assessed until 104 weeks following treatment with rAAV8.aVEGF.

Subjects who meet the inclusion/exclusion criteria are enrolled and receive a 0.5 mg intravitreal injection of ranibizumab in the study eye (Visit 1). At Visit 2 (7 days after ranibizumab injection), subjects are evaluated by SD-OCT to confirm anatomic response to the initial anti-VEGF activity associated with the ranibizumab injection compared with their baseline assessment. Subjects who do not have an anatomic response are withdrawn from the study. For withdrawn subjects, anyone who has an AE associated with the ranibizumab injections on Visit 1 is followed until the AE resolves (up to 30 days post-injection). At Visit 3 (Week 2), subjects receive a single dose of rAAV8.aVEGF Fab administered in an operating room by subretinal delivery. The sentinel subject in each cohort has vision of ≤20/100 and ≥20/400 (≤65 and ≥35 ETDRS letters). After rAAV8.aVEGF Fab administration to the sentinel subject, there is a 4-week observation period for safety. Up to 5 additional subjects (with expanded vision criteria of ≤20/63 and ≥20/400 [≤75 and ≥35 ETDRS letters]) may be enrolled in parallel with a minimum of 1 day between each enrollment. If no safety review triggers (SRTs) are observed, then 4 weeks after the last subject is dosed. Subjects have 3 visits within the first 4 weeks after treatment with rAAV8.aVEGF Fab. Starting 4 weeks after rAAV8.aVEGF Fab administration, subjects may receive intravitreal ranibizumab rescue therapy if they meet predefined rescue injection criteria. Immunogenicity to the vector and transgene of rAAV8.aVEGF Fab is assessed throughout the study.

Safety is the primary focus for the initial 24 weeks after rAAV8.aVEGF administration (primary study period). Following completion of the primary study period, subjects continue to be assessed until 104 weeks following treatment with rAAV8.aVEGF (Week 106). At the end of the study, subjects are invited to participate in a long-term follow-up study. The safety and tolerability of rAAV8.aVEGF are assessed in each dosed subject and are monitored through assessment of ocular and non-ocular AEs and SAEs, chemistry, hematology, coagulation, urinalysis, immunogenicity, ocular examinations and imaging (BCVA, intraocular pressure, slit lamp biomicroscopy, indirect ophthalmoscopy, and SD-OCT), and vital signs.

### A. Arms and Interventions

| Arms | Assigned Intervention |
|---|---|
| Dose 1 | Biological/Vaccine: rAAV8.aVEGF |
| 3 × 10⁹ GC of | is a recombinant adeno-associated |
| rAAV8.aVEGF | virus (AAV) gene therapy vector |
| | carrying a coding sequence for a |
| | soluble anti-VEGF protein |
| Experimental: Dose 2 | Biological/Vaccine: rAAV8.aVEGF |
| 1 × 10¹⁰ GC of | is a recombinant adeno-associated |
| rAAV8.aVEGF | virus (AAV) gene therapy vector |
| | carrying a coding sequence for a |
| | soluble anti-VEGF protein |
| Experimental: Dose 3 | Biological/Vaccine: rAAV8.aVEGF |
| 6 × 10¹⁰ GC of | is a recombinant adeno-associated |
| rAAV8.aVEGF | virus (AAV) gene therapy vector |
| | carrying a coding sequence for a |
| | soluble anti-VEGF protein |

### B. Endpoints:

### Primary Outcome Measure:

1. Safety: Incidence of ocular adverse events (AE) and non-ocular serious adverse events (SAE) over 26 weeks

### Secondary Outcome Measure:

2. Safety: Incidence of ocular and non-ocular AEs and SAEs over 106 weeks
3. Change in best corrected visual acuity (BCVA) over 106 weeks
4. Change in central retinal thickness (CRT) as measured by SD-OCT over 106 weeks.
5. Rescue injections: mean number of rescue injections over 106 weeks
6. Change in choroidal neovascularization and lesion size and leakage area CNV changes as measured by FA over 106 weeks

### Criteria: Inclusion Criteria:

1. Patients ≥50 years with a diagnosis of subfoveal CNV secondary to AMD in the study eye receiving prior intravitreal anti-VEGF therapy. Selected patient population is not gender based (males and females included).
2. BCVA between ≤20/100 and ≥20/400 (≤65 and ≥35 Early Treatment Diabetic Retinopathy Study [ETDRS] letters) for the first patient in each cohort followed by BCVA between ≤20/63 and ≥20/400 (≤75 and ≥35 ETDRS letters) for the rest of the cohort.
3. History of need for and response to anti-VEGF therapy.
4. Response to anti-VEGF at trial entry (assessed by SD-OCT at week 1 (Visit 2)
5. Must be pseudophakic (status post cataract surgery) in the study eye.
6. Aspartate aminotransferase (AST)/Alanine aminotransferase (ALT)<2.5× upper limit of normal (ULN); Total Bilirubin (TB)<1.5×ULN; Prothrombin time (PT)<1.5×ULN; Hemoglobin (Hb)>10 g/dL (males) and >9 g/dL (females); Platelets>100×10³/μL; estimated glomerular filtration rate (eGFR)>30 mL/min/1.73 m²
7. Must be willing and able to provide written, signed informed consent.

### Exclusion Criteria:

1. CNV or macular edema in the study eye secondary to any causes other than AMD.
2. Any condition preventing visual acuity improvement in the study eye, eg, fibrosis, atrophy, or retinal epithelial tear in the center of the fovea.
3. Active or history of retinal detachment in the study eye.
4. Advanced glaucoma in the study eye.
5. History of intravitreal therapy in the study eye, such as intravitreal steroid injection or investigational product, other than anti-VEGF therapy, in the 6 months prior to screening.
6. Presence of an implant in the study eye at screening (excluding intraocular lens).
7. Myocardial infarction, cerebrovascular accident, or transient ischemic attacks within the past 6 months.
8. Uncontrolled hypertension (systolic blood pressure [BP]>180 mmHg, diastolic BP>100 mmHg) despite maximal medical treatment.

### Example 12—Vector Production and Manufacturing

### A. Description of the Manufacturing Process

Cell Seeding: A qualified human embryonic kidney 293 cell line is used for the production process. Cell culture used for vector production is initiated from a single thawed MCB vial, and expanded per a Master Batch Record Document (MBR). Cells are expanded to 5×10⁹-5×10¹⁰ cells using Corning T-flasks and CS-10, which allow sufficient cell mass to be generated for seeding up to 50 HS-36 for vector production per BDS lot. Cells are cultivated in medium composed of Dulbecco's Modified Eagle Medium (DMEM), supplemented with 10% gamma irradiated, US-sourced, Fetal Bovine Serum (FBS). The cells are anchorage dependent and cell disassociation is accomplished using TrypLE™ Select, an animal product-free cell dissociation reagent. Cell seeding is accomplished using sterile, single-use disposable bioprocess bags and tubing sets. The cells are maintained at 37° C. (±2° C.), in 5% (±0.5%) CO₂ atmosphere.

Transient Transfection: Following approximately 3 days of growth (DMEM media+10% FBS), HS-36 cell culture media are replaced with fresh, serum free DMEM media and transfected with the 3 production plasmids using an optimized PEI-based transfection method. All plasmids used in the production process are produced in the context of a CMO quality system and infrastructure utilizing controls to ensure traceability, document control, and materials segregation.

Sufficient DNA plasmid transfection complex are prepared in the BSC to transfect 50 HS-36 (per BDS batch). Initially a DNA/PEI mixture is prepared containing 7.5 mg of the relevant vector genome plasmid), 150 mg of pAdDeltaF6(Kan), 75 mg of pAAV2/8Kan AAV helper plasmid and GMP grade PEI (PEIPro, PolyPlus Transfection SA). This plasmid ratio is determined to be optimal for AAV production in small scale optimization studies. After mixing well, the solution is allowed to sit at room temperature for 25 min, and then added to serum-free media to quench the reaction and then added to the HS-36's. The transfection mixture is equalized between all 36 layers of the HS-36 and the cells are incubated at 37° C. (±2° C.) in a 5% (±0.5%) CO₂ atmosphere for 5 days.

Cell Media Harvesting: Transfected cells and media are harvested from each HS-36 using disposable bioprocess bags by aseptically draining the medium out of the units. Following the harvest of media, the ~200 liter volume is supplemented with MgCl₂ to a final concentration of 2 mM (co-factor for Benzonase) and Benzonase nuclease (Cat #: 1.016797.0001, Merck Group) is added to a final concentration of 25 units/mL. The product (in a disposable bioprocess bag) is incubated at 37° C. for 2 hr in an incubator to provide sufficient time for enzymatic digestion of residual cellular and plasmid DNA present in the harvest as a result of the transfection procedure. This step is performed to minimize the amount of residual DNA in the final vector DP. After the incubation period, NaCl is added to a final concentration of 500 mM to aid in the recovery of the product during filtration and downstream tangential flow filtration.

Clarification: Cells and cellular debris is removed from the product using a depth filter capsule (1.2/0.22 μm) connected in series as a sterile, closed tubing and bag set that is driven by a peristaltic pump. Clarification assures that downstream filters and chromatography columns are protected from fouling and bioburden reduction filtration ensures that at the end of the filter train, any bioburden potentially introduced during the upstream production process is removed before downstream purification. The harvest material is passed through a Sartorius Sartoguard PES capsule filter (1.2/0.22 μm) (Sartorius Stedim Biotech Inc.).

Large-scale Tangential Flow Filtration: Volume reduction (10-fold) of the clarified product is achieved by Tangential Flow Filtration (TFF) using a custom sterile, closed bioprocessing tubing, bag and membrane set. The principle of TFF is to flow a solution under pressure parallel to a membrane of suitable porosity (100 kDa). The pressure differential drives molecules of smaller size through the membrane and effectively into the waste stream while retaining molecules larger than the membrane pores. By recirculating the solution, the parallel flow sweeps the membrane surface preventing membrane pore fouling. By choosing an appropriate membrane pore size and surface area, a liquid sample may be rapidly reduced in volume while retaining and concentrating the desired molecule. Diafiltration in TFF applications involves addition of a fresh buffer to the recirculating sample at the same rate that liquid is passing through the membrane and to the waste stream. With increasing volumes of diafiltration, increasing amounts of the small molecules are removed from the recirculating sample. This results in a modest purification of the clarified product, but also achieves buffer exchange compatible with the subsequent affinity column chromatography step. Accordingly, a 100 kDa, PES membrane is used for concentration that is then diafiltered with a minimum of 4 diavolumes of a buffer composed of: 20 mM Tris pH 7.5 and 400 mM NaCl. The diafiltered product is stored overnight at 4° C. and then further clarified with a 1.2/0.22 μm depth filter capsule to remove any precipitated material.

Affinity Chromatography: The diafiltered product is applied to a Poros™ Capture Select™ AAV8 affinity resin (Life Technologies) that efficiently captures the AAV8 serotype. Under these ionic conditions, a significant percentage of residual cellular DNA and proteins flow through the column, while AAV particles are efficiently captured. Following application, the column is washed to remove additional feed impurities followed by a low pH step elution (400 mM NaCl, 20 mM Sodium Citrate; pH 2.5) that is immediately neutralized by collection into a 1/10^{th} volume of a neutralization buffer (Bis Tris Propane, 200 mM, pH 10.2).

Anion Exchange Chromatography: To achieve further reduction of in-process impurities including empty AAV particles, the Poros-AAV8 elution pool is diluted 50-fold (20 mM Bis Tris Propane, 0.001% Pluronic F68; pH 10.2) to reduce ionic strength to enable binding to a CIMultus™ QA monolith matrix (BIA Separations). Following a low-salt wash, vector product is eluted using a 60 CV NaCl linear salt gradient (10-180 mM NaCl). This shallow salt gradient effectively separates capsid particles without a vector genome (empty particles) from particles containing vector genome (full particles) and results in a preparation enriched for full capsids. Fractions are collected into tubes containing 1/100th volume of 0.1% pluronic F68 and 1/27^{th} volume of Bis Tris pH 6.3 to minimize non-specific binding to tubes and the length of exposure to high pH respectively. The appropriate peak fraction is collected, and the peak area assessed and compared to previous data for determination of the approximate vector yield.

Final Formulation and Bioburden Reduction Filtration to yield the BDS: TFF is used to achieve final formulation on the pooled AEX fractions with a 100 kDa membrane. This is accomplished by diafiltration of formulation buffer (PBS with NaCl and 0.001% Pluronic or PBS with 0.001% Pluronic to be selected following completion of stability studies) and concentrated to yield the BDS Intermediate at a desired target Samples are removed for BDS Intermediate testing (described in the section below). The BDS Intermediate is stored in sterile polypropylene tubes and frozen at ≤−60° C. in a quarantine location until release for Final Fill. Stability studies are underway to assess stability following storage at <−60° C.

Final Fill: The frozen BDS is thawed, pooled, adjusted to the target concentration (dilution or concentrating step via TFF) using the final formulation buffer (PBS with NaCl and 0.001% Pluronic or PBS with 0.001% Pluronic to be selected following completion of stability studies). The product is then be terminally filtered through a 0.22 μm filter and filled into either West Pharmaceutical's “Ready-to-Use” (pre-sterilized) glass vials or Crystal Zenith (polymer) vials (vial type pending the outcome of comparability studies) and stoppers with crimp seals at a fill volume ≥0.1 mL to ≤0.5 mL per vial. Vials are individually labeled according to the specifications below. Labeled vials are stored at ≤−60° C. All doses require dilution in the formulation buffer prior to administration. The dilution is conducted by the pharmacy at the time of dosing.

### B. Assay Methods

Sterility and Bacteriostasis/Fungistasis: This procedure is performed once according to United States Pharmacopeia (USP)<71>, to ensure that the sample matrix does not cause inhibition of the assay. Included in the test is the suitability test.

Particle Aggregation: Drug product particle aggregation is assessed using a dynamic light scattering (DLS) assay. DLS measures fluctuations in scattered light intensity due to diffusing particles and is used to characterize the size of various particles in the sample. DLS instrument software typically displays the particle population at different diameters. If the system is monodisperse, only one population is detected and the mean effective diameter of the particles can be determined. In a polydisperse system, such as in the case of aggregation, multiple particle populations are detected and sized using CONTIN analysis.

Residual plasmid DNA: Detection of plasmid DNA sequences is accomplished using qPCR and primer probe sets specific for the kanamycin gene present in the plasmid backbone but not in vector genomes. The assay is performed in both the presence and absence of DNase digestion such that the amount of free plasmid and the amount packaged into vector particles can be determined.

E1 DNA: Adenoviral E1 DNA is a host cell contaminant and is detected by qPCR specific for the gene. The assay is performed in both the presence and absence of DNase digestion such that both free and packaged E1 DNA can be quantified.

Residual Host Cell DNA: Levels of residual host cell DNA (HCDNA) are quantified using qPCR directed against the human 18s rDNA gene which is a high copy number DNA sequence and thus confers sensitivity. In addition to total residual HCDNA levels, the amount of DNA at various size ranges is also determined.

Residual Host Cell Protein: Residual 293 host cell protein (HCP) is detected using commercially available ELISA kits such as that sold by Cygnus Technologies.

Poros-AAV8 Leachable Ligand: An Enzyme-Linked Immunosorbent Assay (ELISA) kit supplied by Life Technologies, the maker of the Poros-AAV8 resin, is used to detect leached camelid antibody in the drug product.

Mycoplasma Detection: Mycoplasma testing is performed according to USP <63>.

Bioburden Testing: This test is performed according to USP <61>.

Endotoxin Testing: This assay is performed according to USP <85>.

In vitro Assay for Adventitious Agents: The purpose of the in vitro assay for viral contaminants is to detect possible adventitious viruses introduced during AAV8.AMD vector production and is based upon CBER's 1993 Points to Consider and ICH Q5A. The in vitro assays use 3 indicator cell lines-human diploid lung (MRC-5) cells, African green monkey kidney (Vero) cells, and human foreskin fibroblast (Hs68) cells. Assay endpoints are observation of cytopathic effects (CPE) over a course of at least 28 days as well as hemadsorption at the end of the assay period, which facilitates the detection of a broad range of viruses.

Vector Genome Identity: DNA Sequencing: Viral Vector genomic DNA is isolated and the sequence determined by 2-fold sequencing coverage using primer walking. Sequence alignment is performed and compared to the expected sequence.

Vector Capsid Identity: AAV Capsid Mass spectrometry of VP1: Confirmation of the AAV2/8 serotype of the drug product is achieved by an assay based upon analysis of peptides of the AAV capsid protein.

Genomic Copy (GC) Titer: A droplet digital PCR (ddPCR)-based technique for determining the genome copy (GC) titer for AAV vectors is described in Lock et al. Human Gene Therapy Methods 25:115-125. The assay utilized involves digestion with DNase I, followed by digital PCR analysis to measure encapsulated vector genomic copies. DNA detection is accomplished using sequence specific primers targeting the RBG polyA region in combination with a fluorescently tagged probe hybridizing to this same region. A number of standards, validation samples and controls (for background and DNA contamination) have been introduced into the assay.

Empty to Full Particle Ratio: The total particle content of the drug product is determined by SDS-PAGE analysis. A reference vector preparation purified on an iodixanol gradient is analyzed by various methods (analytical ultracentrifugation, electron microscopy and absorbance at 260/280 nm) to established percentage of full particles in the preparation. This reference material is serially diluted to known genome copy numbers (and thus by extension, particle numbers) and each dilution is run on an SDS PAGE gel along with a similar dilution series of the drug product. Peak area volumes of both the reference material and drug product VP3 protein bands are determined by densitometry and the reference material volumes are plotted versus particle number. The total particle concentration of the drug product is determined by extrapolation from this curve and the genome copy (GC) titer is then subtracted to obtain the empty particle titer. The empty to full particle ratio is the ratio of the empty particle titer to the GC titer.

Infectious Titer: The infectious unit (IU) assay is used to determine the productive uptake and replication of AAV8.AMD vector in RC32 cells (rep2 expressing HeLa cells). A 96-well end-point format has been employed similar to that previously published. Briefly, RC32 cells are co-infected by serial dilutions of AAV8.AMD BDS and a uniform dilution of Ad5 with 12 replicates at each dilution of rAAV. Seventy-two hours after infection the cells are lysed, and qPCR performed to detect rAAV vector amplification over input. An end-point dilution Tissue Culture Infectious Dose 50% (TCID50) calculation (Spearman-Karber) is performed to determine a replicative titer expressed as IU/mL. Since “infectivity” values are dependent on particles coming into contact with cells, receptor binding, internalization, transport to the nucleus and genome replication, they are influenced by assay geometry and the presence of appropriate receptors and post-binding pathways in the cell line used. Receptors and post-binding pathways are not usually maintained in immortalized cell lines and thus infectivity assay titers are not an absolute measure of the number of “infectious” particles present. However, the ratio of encapsidated GC to “infectious units” (described as GC/IU ratio) can be used as a measure of product consistency from lot to lot.

Host Cell DNA: A qPCR assay is used to detect residual human 293 DNA. After spiking with a “non-relevant DNA”, total DNA (non-relevant, vector and residual genomic) is extracted from ~1 mL of product. The Host Cell DNA is quantified using qPCR targeting the 18S rDNA gene. The quantities of DNA detected are normalized based on the recovery of the spiked non-relevant DNA.

Host Cell Protein: An ELISA is performed to measure levels of contaminating host HEK293 cell proteins. The Cygnus Technologies HEK293 Host Cell Proteins 2nd Generation ELISA kit is used according to instructions.

Replication-competent AAV (rcAAV) Assay: A sample is analyzed for the presence of replication competent AAV2/8 (rcAAV) that can potentially arise during the production process.

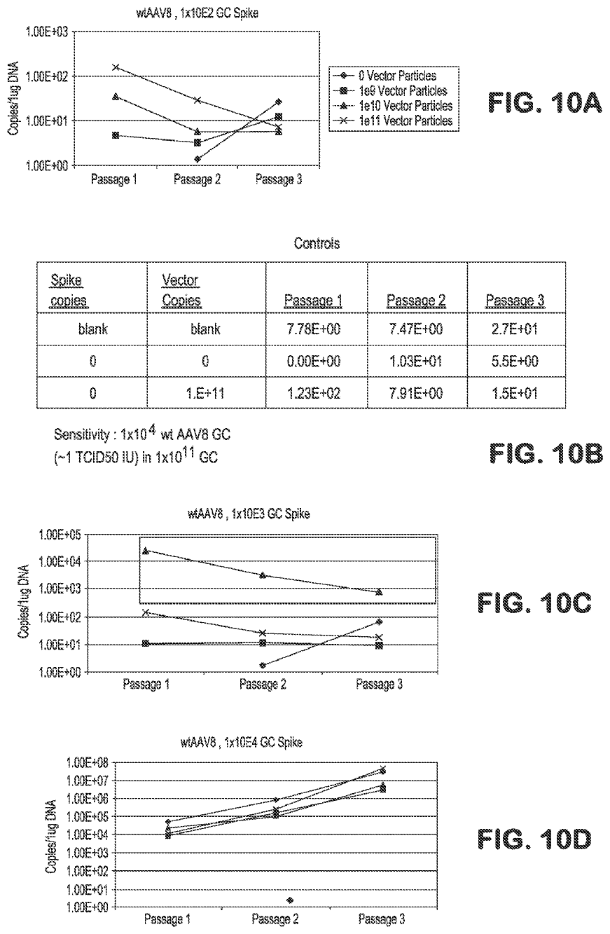

An example of this type of assay is shown in (FIGS. 10A-10D), where wtAAV8 is spiked into different GC amounts of AAV8 vector and the cap gene copy number per 1 μg of 293 cell DNA is determined after 3 successive passages of the cell lysate onto fresh cells. The details of the assay development are included in the CTA submission. These results indicate that the minimum detectable amount of wtAAV8 using this assay is 104 GC. This number is equivalent to approximately 1 TCID50 IU and reflects the lack of infectivity of AAV8 for 293 cells as evidenced by the high GC: IU ratios obtained compared to AAV2. The low sensitivity appears unavoidable with the current assay system but might be overcome in future by engineering a cell line with a yet to be discovered AAV8 cellular receptor or other protein important in post-entry pathways. Spiking the wtAAV8 into AAV8 vector concentrations of up to 10¹¹ GC had little effect on detection and indicates a lack of interference of the vector on wtAAV8 replication at this vector level. While wildtype AAV has been used extensively as a surrogate in the past for rcAAV2 and in our own rcAAV assay development efforts for AAV8, the best surrogate is a AAV8 capsid containing AAV2 ITRs, an AAV2 rep gene and an AAV8 cap gene.

| Sample | | | |
|---|---|---|---|
| Steps | Test | Analytical Method | Acceptance Criteria¹ |
| Final Drug | Appearance | Visual Inspection | Clear to slightly Opaque, |
| Product in | | | colorless to faint white |
| vials | | | solution, free of non- |
| | | | product related foreign |
| | | | particulates |
| | pH | USP <791> | 7.4 +/− 0.2 |
| | GC Titer | ddPCR | ≥1 × 10¹¹ GC/mL** |
| | AAV Vector Genome | Sequencing (Sanger) | Confirm expected sequence |
| | Identity | | |
| Final Drug | Total Protein | Micro BCA | Report result |
| Product in | Content | | |
| vials | Osmolality | USP <785> | <400 mOsm |
| | Content | | |
| | Empty/Full Particle | PT (by SDS-PAGE)/GC | Report result |
| | ratio | Ratio | |
| | Purity | | |
| | Empty:Full particle | OD260/280 | Report result |
| | ratio | | |
| | Purity | | |
| | Viral Capsid Purity | SDS-PAGE | Report result |
| | Purity | | |
| | Aggregation | Dynamic Light Scattering | Report result |
| | Characterization | | |
| | Purity | | |
| | In vitro potency | HEK293transduction/ | Conforms to reference |
| | Potency | ranibizumab ELISA | standard |
| | Transgene expression | In vitro expression and | Positive for ranibizumab |
| | Characterization | ELISA | |
| | Identity | | |
| | Infectious Titer | TCID50/qPCR | Report results |
| | Characterization | | |
| | Potency | | |
| | rcAAV by triple | Cell Culture/qPCR | Report results |
| | passage HEK293 + | | |
| | Ad5 | | |
| | Characterization | | |
| | Safety | | |
| | Capsid Identity | UPLC/Mass Spectrometry | AAV-8 signature peptide |
| | Characterization | | detected. Signature |
| | | | peptides for AAV1, 2, 6, 9 |
| | | | hu37 and Rh10 not |
| | | | detected |
| | Endotoxin | USP <85> | <0.80 EU/mg* |
| | Safety | Kinetic Chromogenic | or |
| | | | (≤the safety limit based on |
| | | | dose calculations, pending |
| | | | the verification of total |
| | | | protein) |
| | Sterility | USP <71> | No Growth |
| | Safety | | |
| | Container Closure | Dye Ingress Test | Container is Integral |
| | Integrity | | |
| | (for stability study | | |
| | only and not for lot | | |
| | release) | | |

¹The acceptance criteria is determined upon completion of the first GMP campaign.
*Endotoxin limit calculation is based on dose in Mass. Once the total protein concentration is confirmed from the GMP run, the limit can be recalculated. The current limit is based on the protein concentration from the Tox materials in relation to GC titer. The value is an approximation and not a definitive value. The dual acceptance criteria presented here.
**DP GC Titer criteria may change depending on the final selected dose levels for the study

The clinically suitable surfactant Pluronic F68 is added to the final formulation buffer of AAV8.AMD and is anticipated to minimize this type of loss. The interaction of the drug product with both the storage vial and the clinical delivery device is investigated to determine the amount of vector loss through binding to surfaces. GC titers (oqPCR) of the engineering run drug product are determined before and after vialling and storage at ≤−60° C. For the delivery device, the DP is thawed, diluted in the appropriate clinical diluent to the correct dosing concentration and passed through the device. GC titrations are performed on the DP directly after thaw, after dilution and after passage through the device, and the appropriate number of replicates is included to assure statistical significance. Comparison of GC titers in this manner enables an assessment of DP loss during storage and administration to the patient. Parallel studies are also performed in a similar way to assess the activity of the drug product after passing through the delivery device. For this purpose the in vitro ranibizumab expression-based potency assay is employed.

### (Sequence Listing Free Text)

The following information is provided for sequences containing free text under numeric identifier <223>.

| SEQ ID NO: | |
|---|---|
| (containing | |
| free text) | Free text under <223> |
| 1 | <223> Humanized anti-VEGF Fab heavy chain |
| | <220> |
| | <221> MISC_FEATURE |
| | <222> (28) . . . (39) |
| | <223> complementarity determining region |
| | <220> |
| | <221> MISC_FEATURE |
| | <222> (54) . . . (83) |
| | <223> complementarity determining region |
| 2 | <223> Humanized anti-VEGF Fab |
| | <220> |
| | <221> MISC_FEATURE |
| | <222> (26) . . . (37) |
| | <223> complementarity determining region |
| | <220> |
| | <221> MISC_FEATURE |
| | <222> (107) . . . (117) |
| | <223> complementarity determining region |
| 3 | <223> 5′ITR.CB7.CI.aVEGFv2.rBG.3′ITR |
| | <220> |
| | <221> repeat_region |
| | <222> (1) . . . (130) |
| | <223> 5′ ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer with 2 mismatches |
| | <220> |
| | <221> misc_feature |
| | <222> (279) . . . (538) |
| | <223> C4 enhancer |
| | <220> |
| | <221> promoter |
| | <222> (582) . . . (862) |
| | <223> CB promoter |
| | <220> |
| | <221> TATA_signal |
| | <222> (836) . . . (839) |
| | <220> |
| | <221> misc_feature |
| | <222> (955) . . . (1829) |
| | <223> chicken beta-actin intron |
| | <220> |
| | <221> Intron |
| | <222> (956) . . . (1928) |
| | <223> chicken beta-actin intron |
| | <220> |
| | <221> Intron |
| | <222> (956) . . . (1928) |
| | <223> chicken beta-actin promoter |
| | <220> |
| | <221> 5′UTR |
| | <222> (1946) . . . (1993) |
| | <223> c-myc 5′ UTR |
| | <220> |
| | <221> misc_feature |
| | <222> (1994) . . . (1999) |
| | <223> kozak sequnce |
| | <220> |
| | <221> transit_peptide |
| | <222> (1999) . . . (2058) |
| | <220> |
| | <221> CDS |
| | <222> (2059) . . . (2427) |
| | <223> aVEGFv2 VH |
| | <220> |
| | <221> CDS |
| | <222> (2428) . . . (2748) |
| | <223> CH1 |
| | <220> |
| | <221> misc_feature |
| | <222> (2752) . . . (2763) |
| | <223> Furin cleavage site |
| | <220> |
| | <221> CDS |
| | <222> (2764) . . . (2835) |
| | <223> F2A linker |
| | <220> |
| | <221> transit_peptide |
| | <222> (2836) . . . (2895) |
| | <220> |
| | <221> CDS |
| | <222> (2896) . . . (3216) |
| | <223> aVEGFv2 VL |
| | <220> |
| | <221> CDS |
| | <222> (3217) . . . (3537) |
| | <223> CL |
| | <220> |
| | <221> polyA_signal |
| | <222> (3613) . . . (3739) |
| | <223> rabbit globin polyA |
| | <220> |
| | <221> repeat_region |
| | <222> (3828) . . . (3957) |
| | <223> 3′ ITR |
| 4 | <223> Synthetic Construct |
| 5 | <223> Synthetic Construct |
| 6 | <223> Synthetic Construct |
| 7 | <223> Synthetic Construct |
| 8 | <223> Synthetic Construct |
| 9 | <213> Artificial Sequence |
| | <220> |
| | <223> |
| | AAV25′ITR.UbC.Ci.aVEGFv2.rBG.AAV23′ITR |
| | <220> |
| | <221> repeat_region |
| | <222> (17) . . . (146) |
| | <223> 5′ ITR |
| | <220> |
| | <221> promoter |
| | <222> (207) . . . (1435) |
| | <223> UbC with C insertion at 289 and G insertion |
| | at 990 |
| | <220> |
| | <221> Intron |
| | <222> (1529) . . . (1661) |
| | <223> chimeric intron |
| | <220> |
| | <221> 5′UTR |
| | <222> (1736) . . . (1783) |
| | <223> c-myc 5′UTR |
| | <220> |
| | <221> misc_feature |
| | <222> (1784) . . . (1789) |
| | <223> kozak |
| | <220> |
| | <221> misc_feature |
| | <222> (1789) . . . (1848) |
| | <223> kozak |
| | <220> |
| | <221> CDS |
| | <222> (1849) . . . (2217) |
| | <223> aVEGFv2 VH |
| | <220> |
| | <221> CDS |
| | <222> (2218) . . . (2538) |
| | <223> CH1 |
| | <220> |
| | <221> misc_feature |
| | <222> (2542) . . . (2553) |
| | <223> furin cleavage site |
| | <220> |
| | <221> misc_feature |
| | <222> (2554) . . . (2625) |
| | <223> F2a linker |
| | <220> |
| | <221> misc_feature |
| | <222> (2626) . . . (2685) |
| | <223> Leader |
| | <220> |
| | <221> CDS |
| | <222> (2686) . . . (3006) |
| | <223> aVEGFv2 VL |
| | <220> |
| | <221> CDS |
| | <222> (3007) . . . (3327) |
| | <223> CL |
| | <220> |
| | <221> polyA_signal |
| | <222> (3345) . . . (3576) |
| | <223> SV40 late polyadenylation signal |
| | <220> |
| | <221> repeat_region |
| | <222> (3641) . . . (3770) |
| | <223> 3′ ITR |
| 10 | <223> Synthetic Construct |
| 11 | <223> Synthetic Construct |
| 12 | <223> Synthetic Construct |
| 13 | <223> Synthetic Construct |
| 14 | <223> ITR.CB7.CI.aVEGRv3.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer with 2 mismatches |
| | <220> |
| | <221> promoter |
| | <222> (582) . . . (862) |
| | <223> CB promoter |
| | <220> |
| | <221> TATA_signal |
| | <222> (836) . . . (839) |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <220> |
| | <221> misc_feature |
| | <222> (1988) . . . (1993) |
| | <223> Kozak |
| | <220> |
| | <221> misc_feature |
| | <222> (1993) . . . (2052) |
| | <223> leader |
| | <220> |
| | <221> CDS |
| | <222> (2053) . . . (2421) |
| | <223> aVEGFv3 VH |
| | <220> |
| | <221> CDS |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2a linker |
| | <220> |
| | <221> misc_feature |
| | <222> (2830) . . . (2889) |
| | <223> leader |
| | <220> |
| | <221> CDS |
| | <222> (2890) . . . (3210) |
| | <223> aVEGFv3 VL |
| | <220> |
| | <221> CDS |
| | <222> (3211) . . . (3531) |
| | <223> CL |
| | <220> |
| | <221> polyA_signal |
| | <222> (3607) . . . (3733) |
| | <223> rabbit globin polyA |
| | <220> |
| | <221> repeat_region |
| | <222> (3822) . . . (3951) |
| | <223> 3′ ITR |
| 15 | <223> Synthetic Construct |
| 16 | <223> Synthetic Construct |
| 17 | <223> Synthetic Construct |
| 18 | <223> Synthetic Construct |
| 19 | <223> ITR.UbC.PI.aVEGFv3.SV40.ITR |
| | <220> |
| | <221> repeat_region |
| | <222> (17) . . . (146) |
| | <223> AAV2 5′ ITR |
| | <220> |
| | <221> promoter |
| | <222> (207) . . . (1434) |
| | <223> UbC, with C insert at 289 and G insert at |
| | 990 |
| | <220> |
| | <221> Intron |
| | <222> (1528) . . . (1660) |
| | <223> chimeric intron |
| | <220> |
| | <221> 5′UTR |
| | <222> (1729) . . . (1776) |
| | <223> c-myc 5′UTR |
| | <220> |
| | <221> misc_feature |
| | <222> (1777) . . . (1782) |
| | <223> kozak |
| | <220> |
| | <221> misc_feature |
| | <222> (1782) . . . (1841) |
| | <223> leader |
| | <220> |
| | <221> CDS |
| | <222> (1842) . . . (2210) |
| | <223> aVEGFv3 VH |
| | <220> |
| | <221> CDS |
| | <222> (2211) . . . (2531) |
| | <223> CH1 |
| | <220> |
| | <221> misc_feature |
| | <222> (2535) . . . (2546) |
| | <223> furin cleavage site |
| | <220> |
| | <221> misc_feature |
| | <222> (2547) . . . (2618) |
| | <223> F2a linker |
| | <220> |
| | <221> misc_feature |
| | <222> (2619) . . . (2678) |
| | <223> leader |
| | <220> |
| | <221> CDS |
| | <222> (2679) . . . (2999) |
| | <223> aVEGFv3 VL |
| | <220> |
| | <221> CDS |
| | <222> (3000) . . . (3320) |
| | <223> CL |
| | <220> |
| | <221> polyA_signal |
| | <222> (3338) . . . (3569) |
| | <223> SV40 late polyA |
| | <220> |
| | <221> repeat_region |
| | <222> (3634) . . . (3763) |
| | <223> AAV2 3′ITR |
| 20 | <223> Synthetic Construct |
| 21 | <223> Synthetic Construct |
| 22 | <223> Synthetic Construct |
| 23 | <223> Synthetic Construct |
| 24 | <223> ITR.UbC.PI.aVEGFv1.SV40.ITR |
| | <220> |
| | <221> repeat_region |
| | <222> (17) . . . (146) |
| | <223> ITR |
| | <220> |
| | <221> promoter |
| | <222> (207) . . . (1435) |
| | <223> UbC |
| | <220> |
| | <221> Intron |
| | <222> (1529) . . . (1661) |
| | <223> Promoga chimeric intron |
| | <220> |
| | <221> 5′UTR |
| | <222> (1730) . . . (1777) |
| | <223> c-myc 5′UTR |
| | <220> |
| | <221> misc_feature |
| | <222> (1778) . . . (1783) |
| | <223> kozak |
| | <220> |
| | <221> misc_feature |
| | <222> (1783) . . . (1842) |
| | <223> leader |
| | <220> |
| | <221> CDS |
| | <222> (1843) . . . (2211) |
| | <223> aVEGFv1 VH |
| | <220> |
| | <221> CDS |
| | <222> (2212) . . . (2532) |
| | <223> CH1 |
| | <220> |
| | <221> misc_feature |
| | <222> (2536) . . . (2547) |
| | <223> furin cleavage site |
| | <220> |
| | <221> misc_feature |
| | <222> (2548) . . . (2619) |
| | <223> F2A linker |
| | <220> |
| | <221> misc_feature |
| | <222> (2620) . . . (2679) |
| | <223> leader |
| | <220> |
| | <221> CDS |
| | <222> (2680) . . . (3000) |
| | <223> aVEGFv1 VL |
| | <220> |
| | <221> CDS |
| | <222> (3001) . . . (3321) |
| | <223> CL |
| | <220> |
| | <221> polyA_signal |
| | <222> (3339) . . . (3570) |
| | <223> SV40 polyadenylation signal |
| | <220> |
| | <221> repeat_region |
| | <222> (3635) . . . (3764) |
| | <223> ITR |
| 25 | <223> Synthetic Construct |
| 26 | <223> Synthetic Construct |
| 27 | <223> Synthetic Construct |
| 28 | <223> Synthetic Construct |
| 30 | <223> synthetic leader |
| 31 | <223> synthetic leader 2 |
| 32 | <223> derived from encephalomycarditts virus |
| 33 | <223> aVEGF |
| | <220> |
| | <221> MISC_FEATURE |
| | <222> (1) . . . (20) |
| | <223> leader |
| | <220> |
| | <221> MISC_FEATURE |
| | <222> (21) . . . (252) |
| | <223> aVEGF Heavy Chain |
| | <220> |
| | <221> MISC_FEATURE |
| | <222> (280) . . . (299) |
| | <223> leader |
| | <220> |
| | <221> MISC_FEATURE |
| | <222> (300) . . . (513) |
| | <223> aVEGF Light Chain |
| 34 | <223> ITR.CB7.CI.aVEGFv1.rBG.ITR |
| | <220> |
| | <221> repeat_region |
| | <222> (1) . . . (130) |
| | <223> 5′ ITR |
| | <220> |
| | <221> promoter |
| | <222> (204) . . . (584) |
| | <223> CMV IE promoter |
| | <220> |
| | <221> promoter |
| | <222> (585) . . . (862) |
| | <223> CB promoter |
| | <220> |
| | <221> TATA_signal |
| | <222> (836) . . . (839) |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv1 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv1 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> Rabbit globin poly A |
| | <220> |
| | <221> misc_feature |
| | <222> (3785) . . . (3821) |
| | <223> part of AAV |
| | <220> |
| | <221> repeat_region |
| | <222> (3822) . . . (3951) |
| | <223> 3′ITR |
| 35 | <223> ITR.CB7.CI.aVEGFv4.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv4 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv4 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> Rabbit globin poly A |
| | <220> |
| | <221> repeat_region |
| | <222> (3822) . . . (3951) |
| | <223> 3′ITR |
| 36 | <223> ITR.CB7.CI.aVEGFv5.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv5 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv5 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> rabbit globin poly A |
| | <220> |
| | <221> repeat_region |
| | <222> (3822) . . . (3951) |
| | <223> 3′ITR |
| 37 | <223> ITR.CB7.CI.aVEGFv6.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv6 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv6 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> Rabbit globin poly A |
| | <220> |
| | <221> repeat_region |
| | <222> (3822) . . . (3951) |
| | <223> 3′ITR |
| 38 | <223> ITR.CB7.CI.aVEGFv7.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv7 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv7 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> rabbit globin poly A |
| | <220> |
| | <221> repeat_region |
| | <222> (3822) . . . (3951) |
| | <223> 3′ITR |
| 39 | <223> ITR.CB7.CI.aVEGFv8.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv8 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv8 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> rabbit globin poly A |
| | <220> |
| | <221> repeat_region |
| | <222> (3822) . . . (3951) |
| | <223> 3′ITR |
| 40 | <223> ITR.CB7.CI.aVEGFv9.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1946) . . . (1993) |
| | <223> c-myc 5′UTR |
| | <220> |
| | <221> misc_feature |
| | <222> (1994) . . . (1999) |
| | <223> kozak |
| | <220> |
| | <221> misc_feature |
| | <222> (1999) . . . (2058) |
| | <223> leader |
| | <220> |
| | <221> misc_feature |
| | <222> (2059) . . . (2427) |
| | <223> aVEGFv9 VH |
| | <220> |
| | <221> misc_feature |
| | <222> (2428) . . . (2748) |
| | <223> CH1 |
| | <220> |
| | <221> misc_feature |
| | <222> (2752) . . . (2763) |
| | <223> furin cleavage site |
| | <220> |
| | <221> misc_feature |
| | <222> (2764) . . . (2835) |
| | <223> F2A linker |
| | <220> |
| | <221> misc_feature |
| | <222> (2836) . . . (2895) |
| | <223> leader |
| | <220> |
| | <221> misc_feature |
| | <222> (2896) . . . (3216) |
| | <223> aVEGFv9 VL |
| | <220> |
| | <221> misc_feature |
| | <222> (3217) . . . (3537) |
| | <223> CL |
| | <220> |
| | <221> misc_feature |
| | <222> (3538) . . . (3543) |
| | <223> Stop Cassette |
| | <220> |
| | <221> polyA_signal |
| | <222> (3613) . . . (3739) |
| | <223> rabbit globin poly A |
| | <220> |
| | <221> repeat_region |
| | <222> (3828) . . . (3957) |
| | <223> 3′ITR |
| 41 | <223> ITR.CB7.CI.aVEGFv10.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv10 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv10 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> rabbit globin poly A |
| | <220> |
| | <221> repeat_region |
| | <222> (3822) . . . (3951) |
| | <223> 3′ITR |
| 42 | <223> ITR.CB7.CI.aVEGFv11.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′ UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv11 VH |
| | <220> |
| | <221> misc_feature |
| | <222> (2422) . . . (2742) |
| | <223> CH1 |
| | <220> |
| | <221> misc_feature |
| | <222> (2746) . . . (2754) |
| | <223> furing cleavage site |
| | <220> |
| | <221> misc_feature |
| | <222> (2755) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv11 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> rabbit globin poly A |
| | <220> |
| | <221> repeat_region |
| | <222> (3822) . . . (3951) |
| | <223> 3′ ITR |
| 43 | <223> ITR.CB7.CI.aVEGFv12.rBG.ITR |
| | <220> |
| | <221> repeat_region |
| | <222> (1) . . . (130) |
| | <223> 5′ITR |
| | <220> |
| | <221> promoter |
| | <222> (198) . . . (579) |
| | <223> CMV IE promoter |
| | <220> |
| | <221> enhancer |
| | <222> (279) . . . (538) |
| | <223> C4 enhancer |
| | <220> |
| | <221> promoter |
| | <222> (582) . . . (862) |
| | <223> CB promoter |
| | <220> |
| | <221> Intron |
| | <222> (956) . . . (1928) |
| | <223> chicken beta-actin intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv12 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furing cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv12 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> rabbit globin poly A |
| | <220> |
| | <221> repeat_region |
| | <222> (3822) . . . (3951) |
| | <223> 3′ITR |
| 44 | <223> ITR.CB7.CI.aVEGFv13.rBG.ITR |
| | <220> |
| | <221> repeat_region |
| | <222> (1) . . . (130) |
| | <223> 5′ITR |
| | <220> |
| | <221> misc_feature |
| | <222> (131) . . . (167) |
| | <223> part of AAV |
| | <220> |
| | <221> promoter |
| | <222> (198) . . . (579) |
| | <223> CME IE promoter |
| | <220> |
| | <221> misc_feature |
| | <222> (204) . . . (233) |
| | <223> promoter start |
| | <220> |
| | <221> enhancer |
| | <222> (279) . . . (538) |
| | <223> C4 enhancer with 2 mismatches |
| | <220> |
| | <221> misc_feature |
| | <222> (561) . . . (584) |
| | <223> CMV promoter end |
| | <220> |
| | <221> promoter |
| | <222> (582) . . . (862) |
| | <223> CB promoter |
| | <220> |
| | <221> misc_feature |
| | <222> (585) . . . (615) |
| | <223> begin promoter |
| | <220> |
| | <221> TATA_signal |
| | <222> (836) . . . (839) |
| | <220> |
| | <221> Intron |
| | <222> (956) . . . (1928) |
| | <223> chichen beta-actin intron |
| | <220> |
| | <221> misc_feature |
| | <222> (1814) . . . (1830) |
| | <223> end of intron |
| | <220> |
| | <221> 5′UTR |
| | <222> (1940) . . . (1987) |
| | <223> c-myc 5′UTR |
| | <220> |
| | <221> misc_feature |
| | <222> (1988) . . . (1993) |
| | <223> kozak |
| | <220> |
| | <221> misc_feature |
| | <222> (1993) . . . (2052) |
| | <223> leader |
| | <220> |
| | <221> misc_feature |
| | <222> (2053) . . . (2421) |
| | <223> aVEGFv13 VH |
| | <220> |
| | <221> misc_feature |
| | <222> (2422) . . . (2742) |
| | <223> CH1 |
| | <220> |
| | <221> misc_feature |
| | <222> (2746) . . . (2757) |
| | <223> furin cleavage site |
| | <220> |
| | <221> misc_feature |
| | <222> (2758) . . . (2829) |
| | <223> F2A linker |
| | <220> |
| | <221> misc_feature |
| | <222> (2830) . . . (2889) |
| | <223> leader |
| | <220> |
| | <221> misc_feature |
| | <222> (2890) . . . (3210) |
| | <223> aVEGFv13 VL |
| | <220> |
| | <221> misc_feature |
| | <222> (3211) . . . (3531) |
| | <223> CL |
| | <220> |
| | <221> misc_feature |
| | <222> (3532) . . . (3537) |
| | <223> stop cassette |
| | <220> |
| | <221> polyA_signal |
| | <222> (3607) . . . (3733) |
| | <223> rabbit globin poly A |
| | <220> |
| | <221> misc_feature |
| | <222> (3785) . . . (3821) |
| | <223> part of AAV |
| | <220> |
| | <221> repeat_region |
| | <222> (3822) . . . (3951) |
| | <223> 3′ITR |
| 45 | <223> |
| | ITR.CMV.PI.aVEGFv7.eMCV.IRES.SV40.ITR |
| | <220> |
| | <221> repeat_region |
| | <222> (1) . . . (130) |
| | <223> ITR |
| | <220> |
| | <221> promoter |
| | <222> (191) . . . (932) |
| | <223> human CMV I.E. enhancer & promoter |
| | <220> |
| | <221> Intron |
| | <222> (1047) . . . (1179) |
| | <223> Promega chimeric intron |
| | <220> |
| | <221> 5′UTR |
| | <222> (1248) . . . (1295) |
| | <223> c-myc 5′UTR |
| | <220> |
| | <221> misc_feature |
| | <222> (1299) . . . (1307) |
| | <223> kozak |
| | <220> |
| | <221> misc_feature |
| | <222> (1305) . . . (1364) |
| | <223> leader |
| | <220> |
| | <221> misc_feature |
| | <222> (1365) . . . (1685) |
| | <223> aVEGFv7 VL |
| | <220> |
| | <221> misc_feature |
| | <222> (1686) . . . (2006) |
| | <223> CL |
| | <220> |
| | <221> enhancer |
| | <222> (2018) . . . (2608) |
| | <223> IRES |
| | <220> |
| | <221> misc_feature |
| | <222> (2606) . . . (2665) |
| | <223> leader |
| | <220> |
| | <221> misc_feature |
| | <222> (2666) . . . (3034) |
| | <223> aVEGFv7 VH |
| | <220> |
| | <221> misc_feature |
| | <222> (3035) . . . (3355) |
| | <223> CH1 |
| | <220> |
| | <221> polyA_signal |
| | <222> (3384) . . . (3615) |
| | <223> SV40 late polyadenylation signal |
| | <220> |
| | <221> repeat_region |
| | <222> (3680) . . . (3809) |
| | <223> ITR |
| 46 | <223> |
| | ITR.CMV.PI.aVEGFv7.fmdIRES.SV40.ITR |
| | <220> |
| | <221> repeat_region |
| | <222> (1) . . . (130) |
| | <223> ITR |
| | <220> |
| | <221> promoter |
| | <222> (191) . . . (932) |
| | <223> human CMV I.E. enhancer and promoter |
| | <220> |
| | <221> TATA_signal |
| | <222> (897) . . . (901) |
| | <220> |
| | <221> Intron |
| | <222> (1047) . . . (1179) |
| | <223> Promega chimeric intron |
| | <220> |
| | <221> 5′UTR |
| | <222> (1248) . . . (1295) |
| | <223> c-myc 5′UTR |
| | <220> |
| | <221> misc_feature |
| | <222> (1299) . . . (1307) |
| | <223> kozak |
| | <220> |
| | <221> misc_feature |
| | <222> (1305) . . . (1313) |
| | <223> leader |
| | <220> |
| | <221> misc_feature |
| | <222> (1314) . . . (1364) |
| | <223> leader |
| | <220> |
| | <221> misc_feature |
| | <222> (1365) . . . (1685) |
| | <223> aVEGFv7 VL |
| | <220> |
| | <221> misc_feature |
| | <222> (1686) . . . (2006) |
| | <223> CL |
| | <220> |
| | <221> misc_feature |
| | <222> (2021) . . . (2482) |
| | <223> FMDV |
| | <220> |
| | <221> misc_feature |
| | <222> (2501) . . . (2542) |
| | <223> leader |
| | <220> |
| | <221> misc_feature |
| | <222> (2543) . . . (2911) |
| | <223> aVEGFv7 VH |
| | <220> |
| | <221> misc_feature |
| | <222> (2912) . . . (3232) |
| | <223> CH1 |
| | <220> |
| | <221> polyA_signal |
| | <222> (3261) . . . (3492) |
| | <223> Sv40 late polyadenylation signal |
| | <220> |
| | <221> repeat_region |
| | <222> (3557) . . . (3686) |
| | <223> ITR |
| 47 | <223> |
| | ITR.CMV.PI.aVEGFv7.cMycIRES.SV40.ITR |
| | <220> |
| | <221> repeat_region |
| | <222> (1) . . . (130) |
| | <223> ITR |
| | <220> |
| | <221> promoter |
| | <222> (191) . . . (932) |
| | <223> human CMV I.E. enhancer and promoter |
| | <220> |
| | <221> TATA_signal |
| | <222> (897) . . . (901) |
| | <220> |
| | <221> Intron |
| | <222> (1047) . . . (1179) |
| | <223> Promega chimeric intron |
| | <220> |
| | <221> 5′UTR |
| | <222> (1248) . . . (1295) |
| | <223> c-myc 5′UTR |
| | <220> |
| | <221> misc_feature |
| | <222> (1299) . . . (1307) |
| | <223> kozak |
| | <220> |
| | <221> misc_feature |
| | <222> (1305) . . . (1313) |
| | <223> leader |
| | <220> |
| | <221> misc_feature |
| | <222> (1314) . . . (1364) |
| | <223> leader |
| | <220> |
| | <221> misc_feature |
| | <222> (1365) . . . (1685) |
| | <223> aVEGFv7 VL |
| | <220> |
| | <221> misc_feature |
| | <222> (1686) . . . (2006) |
| | <223> CL |
| | <220> |
| | <221> misc_feature |
| | <222> (2021) . . . (2415) |
| | <223> IRES c-myc |
| | <220> |
| | <221> misc_feature |
| | <222> (2226) . . . (2273) |
| | <223> mini c-myc IRES |
| | <220> |
| | <221> misc_feature |
| | <222> (2275) . . . (2275) |
| | <223> this C to T mutaion increases expression |
| | <220> |
| | <221> misc_feature |
| | <222> (2434) . . . (2475) |
| | <223> leader |
| | <220> |
| | <221> misc_feature |
| | <222> (2476) . . . (2844) |
| | <223> aVEGFv7 VH |
| | <220> |
| | <221> misc_feature |
| | <222> (2845) . . . (3165) |
| | <223> CH1 |
| | <220> |
| | <221> polyA_signal |
| | <222> (3194) . . . (3425) |
| | <223> SV40 late polyadenylation signal |
| | <220> |
| | <221> repeat_region |
| | <222> (3490) . . . (3619) |
| | <223> ITR |
| 48 | <223> AAV8 capsid |
| 49 | <223> Nucleic acid sequence of AAV8 capsid |

All publications cited in this specification are incorporated herein by reference in their entireties, as are U.S. Provisional Patent Application No. 62/466,721, filed Mar. 3, 2017, U.S. Provisional Patent Application No. 62/460,515, filed Feb. 17, 2017, U.S. Provisional Patent Application No. 62/442,946, filed Jan. 5, 2017, U.S. Provisional Patent Application No. 62/331,100, filed May 3, 2016 and U.S. Provisional Patent Application No. 62/323,184, filed Apr. 15, 2016. Similarly, the Sequence Listing filed herewith is hereby incorporated by reference. While the invention has been described with reference to particular embodiments, it will be appreciated that modifications can be made without departing from the spirit of the invention. Such modifications are intended to fall within the scope of the appended claims.

1-20. (canceled)

21. A method of delivering a liquid suspension comprising a recombinant adeno-associated virus (rAAV) to a subject in need thereof, comprising subretinally administering the liquid suspension comprising the rAAV comprising an anti-VEGF transgene resulting in distribution of an anti-VEGF transgene expression product outside an injection bleb, wherein the rAAV comprises an AAV8 capsid comprising a vector genome wherein the vector genome comprises a coding sequence for an anti-human vascular endothelial growth factor (VEGF) antigen binding antibody fragment (Fab) having an exogenous leader sequence, a heavy immunoglobulin chain, a linker, and a light immunoglobulin chain having an exogenous leader sequence, wherein the coding sequence is operably linked to regulatory elements which direct expression of the anti-VEGF Fab in the eye.

22. The method of claim 21, wherein subretinally administering the rAAV further results in distribution of the anti-VEGF Fab throughout the retina.

23. The method according to claim 22, wherein subretinally administering the rAAV further results in expression of the anti-VEGF Fab in vitreous and anterior chamber fluids.

24. The method of claim 22, wherein a single administration of the rAAV results in continuous delivery of an effective amount of the anti-VEGF Fab throughout the retina.

25. The method of claim 21, wherein the patient has wet age-related related macular degeneration.

26. The method of claim 25, wherein the vector genome comprises a 5′ AAV inverted terminal repeat (ITR) and a 4′ AAV ITR.

27. The method of claim 25, wherein the vector genome comprises regulatory elements which direct expression of the heavy and light immunoglobulin chains of the anti-VEGF Fab.

28. The method of claim 25, wherein the linker is an F2A linker.

29. The method of claim 22, wherein the rAAV are delivered in a volume of about 75 μL to about 150 μL of the suspension.

30. The method of claim 29, wherein the rAAV are delivered in a volume of about 100 μL of the suspension.
